(19) United States
(12) Patent Application Publication
(10) Pub. No.: US 2022/0339138 A1
(43) Pub. Date: Oct. 27, 2022

(54) METHODS AND COMPOSITIONS FOR TREATMENT OF RETT SYNDROME

(71) Applicants: ACADIA PHARMACEUTICALS INC., SAN DIEGO, CA (US); NEUREN PHARMACEUTICALS LIMITED, AUCKLAND (NZ)

(72) Inventors: MONA DARWISH, MALVERN, PA (US); JAMES M. YOUAKIM, BRYN MAWR, PA (US); LAWRENCE IRWIN GLASS, TAKOMA PARK, MD (US); NANCY ELIZABETH JONES, JONGNY (CH); SEAN PAUL OOSTERHOLT, KINGSTON UPON THAMES (GB); OSCAR DELLA PASQUA, LONDON (GB)

(21) Appl. No.: 17/590,496

(22) Filed: Feb. 1, 2022

Related U.S. Application Data
(63) Continuation of application No. PCT/US2020/057627, filed on Oct. 28, 2020.
(60) Provisional application No. 63/031,201, filed on May 28, 2020.
(60) Provisional application No. 62/927,008, filed on Oct. 28, 2019.

(51) Int. Cl. A61K 31/40; A61K 9/00; A61P 25/28

(52) U.S. Cl. CPC A61K 31/40; A61K 9/0053; A61P 25/28

(57) ABSTRACT

Disclosed herein are methods of treating Rett syndrome comprising administering trofinetide to a subject in need thereof in which a dosage is provided that may reduce or avoid underexposure, e.g., in low body weight subjects, and/or provide other benefits.

### BACKGROUND OF THE INVENTION

### Field of Invention

The present disclosure provides methods of treating Rett syndrome comprising administering trofinetide to a subject in need thereof.

### Background

Rett syndrome (RTT) is a seriously debilitating neurodevelopmental disorder for which there is currently no approved treatment. Its prevalence is reported as 1 in 10,000-15,000 female live births (Bienvenu et al., “The incidence of Rett syndrome in France,” Pediatr. Neurol. 2006, 34(5), 372-375; Neul et al., “Rett syndrome: revised diagnostic criteria and nomenclature,” Ann. Neurol. 2010, 68(6), 944-950). While the great majority of patients with RTT are females, males who meet the criteria for RTT have also been identified (Neul et al., “The array of clinical phenotypes of males with mutations in Methyl-CpG binding protein 2,” Am. J. Genet. B. Neuropsychiatr. Genet. 2019, 180(1), 55-67).

Rett syndrome includes “typical” and “atypical” forms, also sometimes referred to as “classic” and “variant” RTT, respectively. Atypical RTT generally involves some but not all of the criteria required for a typical RTT diagnosis and is discussed in more detail below. In patients with typical RTT, there is seemingly normal psychomotor development for the first six months of life, but soon thereafter the failure to reach normal developmental milestones is observed, followed by a period of developmental regression in which there is a loss of normal use of the hands and of spoken language, and a loss or impairment of ambulation (Samaco and Neul, “Complexities of Rett syndrome and MeCP2, J. Neurosci. 2011, 31(22), 7951-7959). The period of developmental regression is accompanied by transient autistic features in many but not all individuals with RTT (Lee et al., “Early development and regression in Rett syndrome, Clin. Genet. 2013, 84(6), 572-576; Neul et al., “Developmental delay in Rett syndrome: data from the natural history study,” J. Neurodevelop. Dis. 2014, 6(20), 1-9). Loss of social skills appears to stabilize or reverse after the regression period, and some individuals with RTT demonstrate social intentions through eye contact (Young et al., “The diagnosis of autism in a female: could it be Rett syndrome? Eur. J. Pediatr. 2008, 167(6), 661-669; Djukic and McDermott “Social preferences in Rett syndrome,” Pediatr. Neurol. 2012, 46(4), 240-242; Urbanowicz et al., “An exploration of the use of eye gaze and gestures in females with Rett syndrome,” J. Speech Lang. Hear. Res. 2016, 59(6), 1373-1383). Nonetheless, social interaction and communication remain limited (Mount et al., “Features of autism in Rett syndrome and severe mental retardation,” J. Autism Dev. Disord. 2003, 33(4), 435-442; Urbanowicz et al., “Aspects of speech language abilities are influenced by MECP2 mutation type in girls with Rett syndrome,” Am. J. Med. Genet. 2015, 167A(2), 354-362; Rose et al., “Rett syndrome: an eye-tracking study of attention and recognition memory,” Dev. Med. Child Neurol. 2013, 55(4), 364-371; Kaufmann et al., “Social impairments in Rett syndrome: characteristics and relationship to clinical severity,” J. Intellect. Disabil. Res. 2012, 56(3), 233-247; Woodyatt and Ozanne, “A longitudinal study of cognitive skills and communication behaviours in children with Rett syndrome,” J. Intellect. Disabil. Res. 1993, 37(Pt r), 419-435). The intellectual disability in RTT appears to be profound; however, precise measurement of the extent of cognitive impairment is difficult because of the severe communication and motor deficits affecting most individuals (Byiers and Symons, “Issues in estimating developmental level and cognitive function in Rett syndrome,” In: Hodapp, R. M. (ed.), International Review of Research in Intellectual and Developmental Disabilities, Waltham, Mass.: Elsevier Inc.; 2012, 43, 147-185; Clarkson et al., “Adapting the Mullen Scales of Early Learning for a standardized measure of cognition in children with Rett syndrome,” Intellect. Dev. Disabil. 2017, 55(6), 419-431).

Seizures are common, although not diagnostic. Commonly observed symptoms include awake breathing disruptions, scoliosis, and interest in social interaction (intense eye communication) (Neul 2010, supra; Percy el al., “Profiling scoliosis in Rett syndrome,” Pediatr. Res. 2010, 67(4), 436-439). Gastrointestinal symptoms, including constipation and chewing and swallowing difficulties are observed in the majority of patients with RTT (Motil et al., “Gastrointestinal and nutritional problems occur frequently throughout life in girls and women with Rett syndrome,” J. Pediatr. Gastroenterol. Nutr. 2012, 55(3, 292-298). Sudden changes in mood, screaming and inconsolable crying are common behaviors in children and adolescents with RTT (Mount et al., “Behavioural and emotional features in Rett syndrome,” Disabil. Rehabil. 2001, 23(3-4), 129-138; Mount et al., “The Rett Syndrome Behaviour Questionnaire (RSBQ): Refining the behavioural phenotype of Rett syndrome,” J. Child Psychol. Psychiatry 2002, 43(8), 1099-1110; Robertson et al., “The association between behavior and genotype in Rett syndrome using the Australian Rett syndrome database,” Am. J. Med. Genet. 2006, 141B(2), 177-183; Cianfaglione et al., “A national survey of Rett syndrome: behavioural characteristics,” J. Neurodev. Disord. 2015, 7(1), 11). These impairments can further exacerbate other symptoms and disrupt activities of daily living including educational, recreational and treatment opportunities (Epstein et al., “Conceptualizing a quality of life framework for girls with Rett syndrome using qualitative methods,” Am. J. Med. Genet. A 2016, 170(3), 645-653; Perry et al., “Brief report: cognitive and adaptive functioning in 28 girls with Rett syndrome,” J. Autism Dev. Disord. 1991, 21(4), 551-556; Thompson and Iwata, “A descriptive analysis of social consequences following problem behavior,” J. Appl. Behav. Anal. 2001, 34(2), 169-178; Iemmi et al., “Positive behavioural support for children and adolescents with intellectual disabilities whose behavior challenges: an exploration of the economic case,” J. Intellect. Disabil. 2015, 20(3), 281-295). Autonomic manifestations, which include abnormalities in cardiac and respiratory function, as well as in peripheral circulation, are considered to be predominantly of CNS origin. RTT is also characterized by impaired growth affecting the brain and other organ systems.

Loss of purposeful hand use is commonly observed in subjects with RTT during the onset of regression and at least 30% of individuals remain without any type of purposeful hand use (Downs et al., “Level of purposeful hand function as a marker of clinical severity in Rett syndrome,” Dev. Med. Child Neurol. 2010, 52(9), 817-823). Individuals with some purposeful hand movements vary in their level of ability to reach for and grasp objects. The ability to self-feed is observed in 25-43% (Cass et al., “Findings from a multidisciplinary clinical case series of females with Rett syndrome,” Dev. Med. Child Neurol. 2003, 45, 325-337; Larsson et al., “Rett syndrome from a family perspective: the Swedish Rett Center Survey,” Brain Dev. 2005, 27 Suppl 1, S14-19; Downs et al. 2011). Poorer levels of hand function are associated with both age and severity of mobility impairment (Downs et al., “Longitudinal hand function in Rett syndrome,” J. Child Neurol. 2011, 26(3), 334-340).

In adulthood, a late motor deterioration stage occurs, characterized by worsening dystonia, rigidity, and in some cases, deterioration in the ability to walk and parkinsonian symptoms. Affected individuals have a yearly death rate between 1 and 2%, with 25% of all deaths characterized as sudden and unexpected (Kerr el al., “Rett syndrome: analysis of deaths in the British survey,” Eur. Child Adolesc. Psychiatry 1997, 6 Suppl 1, 71-74; Samaco and Neul 2011, supra). Women with RTT can survive into their fifties and occasionally longer (Samaco and Neul 2011, supra).

There are no medicines approved for the treatment of Rett syndrome. Current treatment options focus on the management of each patient's symptoms and, even in that regard, is often unsatisfactory and has only a limited effect on functional improvement. Accordingly, the burden on families and other caregivers is significant (Palacios-Ceña et al., “‘Living an obstacle course’: A qualitative study examining the experiences of caregivers of children with Rett syndrome,” Int. J. Environ. Res. Public Health 2018, 16(1), 41).

In 96-98% of patients diagnosed with classic RTT, the disease is caused by mutations in the X-linked MECP2 gene (Percy et al., “When Rett syndrome is due to genes other that MECP2,” Transl. Sci. Rare Dis. 2018, 3(1), 49-53). MECP2 encodes methyl-CpG binding protein 2 (MeCP2), which modulates gene expression by binding to methylated CpG dinucleotides, primarily by activating but also by repressing transcription (Ip et al., “Rett syndrome: insights into genetic, molecular and circuit mechanisms,” 2018, 19(6), 368-382; Neul et al., “Specific mutations in methyl-CpG-binding protein 2 confer different severity in Rett syndrome,” Neurology 2008, 70(16), 1313-1321; Kriaucionis et al., “DNA methylation and Rett syndrome,” Hum. Mol. Genel. 2003, 12 Spec No 2, R221-227; Hite el al., “Recent advances in MeCP2 structure and function,” Biochem. Cell Biol. 2009, 87(1), 219-227). The activity of the MeCP2 protein is diminished in both neurons and astrocytes (Yasui et al., “MeCP2 modulates gene expression pathways in astrocytes,” Mol. Autism 2013, 4(1), 3). This protein is critical for development of the nervous system, particularly the brain. The mutations reduce production, or cause inaccurate production, of the protein. Without sufficient levels of working protein, the brain does not develop normally, leading to Rett syndrome. There are eight MECP2 mutations that make up the majority of Rett syndrome cases, and the location and type of mutation impact the development and severity of syndrome symptoms, with MECP2 T158M and MECP2 R106W mutations among the most prevalent (Amir et al., “Rett syndrome is caused by mutations in X-linked MECP2,” Nature Genetics 1999, 23(2), 185-188; Percy et al., “Rett syndrome: North American database,” J. Child Neurol. 2007, 22(12), 1338-1341).

Trofinetide is a synthetic analog of glycine-proline-glutamate (also known as glypromate or GPE), a peptide that occurs naturally in the brain. GPE is the n-terminal tripeptide of the insulin-like growth factor 1 (IGF-1) protein. The structure of trofinetide is shown below.

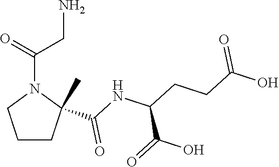 [SMILES: C[C@@]1(C(=O)N[C@@H](CCC(=O)O)C(=O)O)CCCN1C(=O)CN]

Trofinetide crosses the blood-brain barrier following oral administration. In the brain, it is believed to normalize decreased bioavailability of IGF-1 and GPE, as well as having an anti-inflammatory effect on pathologically activated glial cells. Both conditions contribute to deficits in synaptic development and functional maturation of synaptic plasticity that are fundamental to the wide-ranging effects of RTT. Mechanistically, trofinetide is postulated to diminish neuroinflammation, reduce microglial activation and astrogliosis, normalize protein synthesis and dendritic morphology, and restore homeostasis of excitatory and inhibitory neuronal signaling (Lu et al., “Glycyl-L-2-methylprolyl-L-glutamic acid, a glypromate analog, attenuates brain ischemia-induced non-convulsive seizures in rats,” J. Cerebr. Blood Flow & Metab. 2009, 29, 1924-1932; Wei et al., “Glycyl-L-2-methylprolyl-L-glutamic acid treatment inhibits neuroinflammation and pro-inflammatory cytokine expression induced by experimental penetrating ballistic-like brain injury in rats,” J. Neuroinflammation 2009, 6, 19; Deacon et al., “NNZ-2566, a novel analog of (1-3) IGF-1, as a potential therapeutic agent for fragile X syndrome,” Neuromol. Med. 2015, 17, 1). Treatment with GPE reversed Rett-like symptoms in MeCP2 mutant mice (Tropea et al., “Partial reversal of Rett syndrome-like symptoms in MeCP2 mutant mice,” Proc. Natl. Acad. Sci. USA 2009, 106, 2029-2034). Therefore, trofinetide represents an opportunity in the treatment of Rett syndrome. Such treatment may exert an effect on brain structure and function such as dendritic length and branching and long-term potentiation, which would be expected to lead to improvements across a wide range of symptoms of RTT.

Pharmacokinetic data is available from previous clinical trials of trofinetide in healthy subjects and clinical trials of trofinetide in subjects with Rett syndrome (the first of those in adolescents and adults and the second of those in children and adolescents) (Oosterholt et al., “Population pharmacokinetics of NNZ-2566 in healthy subjects,” Eur. J. Pharm. Sci. 2017, 15, 109S, S98-107; Glaze et al., “A double-blind, randomized, placebo-controlled study of trofinetide in pediatric Rett syndrome,” Neurology 2019, 92(16), e1912-1925). However, a significant proportion of subjects in the interventional clinical trials did not achieve exposure of trofinetide at levels anticipated to be effective.

### BRIEF SUMMARY OF THE INVENTION

As discussed in detail herein, analysis of the results from human clinical studies indicates that lower than anticipated exposure of trofinetide is associated with pediatric subjects having lower body weights. The presently disclosed methods involve different dosing regimens with the objective of mitigating this problem and/or improving trofinetide exposure levels in subjects, providing other benefits, or at least providing the public with a useful choice. In particular, Applicants have surprisingly discovered that high doses of trofinetide can be safely administered to pediatric subjects having low body weights to treat Rett syndrome.

In one aspect, the present disclosure provides a method of treating Rett syndrome in a subject in need thereof, the method comprising administering a therapeutically effective amount of trofinetide to the subject in a daily amount of:
a) 4-10.0 g if the subject weighs between 8-11.9 kg;
b) 10.1-14.0 g if the subject weighs between 12.0-20.0 kg;
c) 14.1-18.0 g if the subject weighs between 20.1-35.0 kg;
d) 18.1-22.0 g if the subject weighs between 35.1-50.0 kg; or
e) 22.1-26 g if the subject weighs between 50.1-150 kg.

In another aspect, the present disclosure provides trofinetide for use in treating Rett syndrome in a subject, wherein trofinetide is to be administered in a daily amount of:
a) 4-10.0 g if the subject weighs between 8-11.9 kg;
b) 10.1-14.0 g if the subject weighs between 12.0-20.0 kg;
c) 14.1-18.0 g if the subject weighs between 20.1-35.0 kg;
d) 18.1-22.0 g if the subject weighs between 35.1-50.0 kg; or
e) 22.1-26 g if the subject weighs between 50.1-150 kg.

In another aspect, the present disclosure provides the use of trofinetide for the manufacture of a medicament for treating Rett syndrome in a subject, wherein trofinetide is to be administered in a daily amount of:
a) 4-10.0 g if the subject weighs between 8-11.9 kg;
b) 10.1-14.0 g if the subject weighs between 12.0-20.0 kg;
c) 14.1-18.0 g if the subject weighs between 20.1-35.0 kg;
d) 18.1-22.0 g if the subject weighs between 35.1-50.0 kg; or
e) 22.1-26 g if the subject weighs between 50.1-150 kg.

### BRIEF DESCRIPTION OF THE DRAWINGS

FIG. 1 shows a graph of the clearance (Cl) of trofinetide following dosing in adolescents and adults with RTT compared with healthy volunteers (three combined data sets) as a function of body weight.

FIG. 2 shows a graph of the clearance (Cl) of trofinetide following dosing in children and adolescents with RTT as a function of body weight.

FIG. 3 shows a graph of the central volume of distribution (Vc) of trofinetide following dosing in adolescents and adults with RTT compared with healthy volunteers (three combined data sets) as a function of body weight.

FIG. 4 shows a graph of the central volume of distribution (Vc) of trofinetide following dosing in children and adolescents with RTT as a function of body weight.

FIGS. 5A-D are graphs showing the relationship between percentage change from treatment baseline in RSBQ total score (RBTOT) and trofinetide AUCss at Day 28 by visit (Visit 5, FIG. 5A; Visit 6, FIG. 5B; Visit 7, FIG. 5C; Visit 8, FIG. 5D).

FIG. 6 is a graph showing the relationship between percentage change from treatment baseline in RSBQ total score (RBTOT) and trofinetide cumulative AUC₀₋ₓ during the active trofinetide dosing period.

FIG. 7 is a bar graph showing the distribution of AUC results for the 200 mg/kg dosing arm in the prior trofinetide study in children and adolescents.

FIG. 8 is a graph showing simulated AUC profiles for dosing trofinetide at 200 mg/kg at a consistent dosing of 10,000 mg BID.

FIG. 9 is a graphical summary of timing of assessments and assessors.

FIG. 10 is a graph showing the predicted probabilities of CGI-I scores over a range of Day 42 Cₘₐₓ values of 0 to 600 μg/mL.

FIG. 11 is a graph showing the predicted change in RSBQ scores versus AUC₀₋₁₂ on Day 42 of treatment with trofinetide.

FIG. 12 is a is a boxplot summarizing the predicted Day 14 AUC₀₋₁₂ values for each body weight band following the estimated dosing regimens of trofinetide.

### DETAILED DESCRIPTION

Reference will now be made in detail to certain embodiments of the invention. While the invention will be described in conjunction with the described embodiments, it will be understood that such descriptions are not intended to limit the invention to those embodiments. On the contrary, the invention is intended to cover all alternatives, modifications, and equivalents, which may be included within the invention as defined by the appended claims.

Before describing the present teachings in detail, it is to be understood that the disclosure is not limited to specific compositions or process steps, as such may vary. It should be noted that, as used in this specification and the appended claims, the singular form “a,” “an,” and “the” include plural references unless the context clearly dictates otherwise.

“Or” is used in the inclusive sense, i.e., equivalent to “and/or,” unless the context requires otherwise.

Numeric ranges are inclusive of the numbers defining the range. Measured and measurable values are understood to be approximate, taking into account significant digits and the error associated with the measurement.

Also, the use of “comprise,” “comprises,” “comprising,” “contain,” “contains,” “containing,” “include,” “includes,” “included,” and “including” are not intended to be limiting. It is to be understood that both the foregoing general description and detailed description are exemplary and explanatory only and are not restrictive of the teachings.

Unless specifically noted in the above specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims).

The term “trofinetide” as used herein refers to glycyl-L-2-methylprolyl-L-glutamic acid of Formula I:

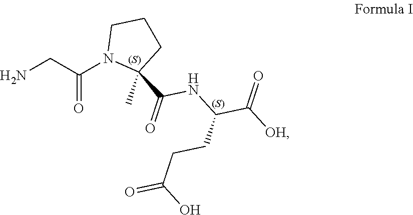

or “G-2-MePE,” “H-Gly-MePro-Glu-OH,” or “Gly-MePro-Glu-OH,” wherein each stereocenter is in the S configuration, or a pharmaceutically acceptable salt thereof. The IUPAC name of trofinetide is (2S)-2-[[(2S)-1-(2-aminoacetyl)-2-methylpyrrolidine-2-carbonyl]amino]pentanedioic acid.

In one embodiment, the term trofinetide refers to a compound of Formula I, i.e., it is a neutral species.

In another embodiment, the term trofinetide refers to a pharmaceutically acceptable salt of a compound of Formula I.

The term “pharmaceutically acceptable salt” as used herein, refers to any salt, e.g., a salt obtained by reaction with an acid or a base, of Formula I that is physiologically tolerated in a subject. Pharmaceutically acceptable salts include, but are not limited to, metal salts such as sodium salt, potassium salt, cesium salt and the like; alkaline earth metals such as calcium salt, magnesium salt and the like; organic amine salts such as triethylamine salt, pyridine salt, picoline salt, ethanolamine salt, triethanolamine salt, dicyclohexylamine salt, N,N′-dibenzylethylenediamine salt and the like; inorganic acid salts such as hydrochloride, hydrobromide, phosphate, sulphate and the like; organic acid salts such as citrate, lactate, tartrate, maleate, fumarate, mandelate, acetate, dichloroacetate, trifluoroacetate, oxalate, formate and the like; sulfonates such as methanesulfonate, benzenesulfonate, p-toluenesulfonate and the like; and amino acid salts such as arginate, asparginate, glutamate and the like.

Acid addition salts can be formed by mixing a solution of Formula I with a solution of a pharmaceutically acceptable non-toxic acid such as hydrochloric acid, fumaric acid, maleic acid, succinic acid, acetic acid, citric acid, tartaric acid, carbonic acid, phosphoric acid, oxalic acid, dichloroacetic acid, or the like. Basic salts can be formed by mixing a solution of Formula I with a solution of a pharmaceutically acceptable non-toxic base such as sodium hydroxide, potassium hydroxide, choline hydroxide, sodium carbonate and the like.

The terms “or a combination thereof” and “or combinations thereof” as used herein refers to any and all permutations and combinations of the listed terms preceding the term. For example, “A, B, C, or combinations thereof” is intended to include at least one of: A, B, C, AB, AC, BC, or ABC, and if order is important in a particular context, also BA, CA, CB, ACB, CBA, BCA, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

Unless otherwise defined herein, scientific and technical terms used herein have the meanings that are commonly understood by those of ordinary skill in the art. In the event of any latent ambiguity, definitions provided herein take precedence over any dictionary or extrinsic definition. Unless otherwise required by context, singular terms shall include pluralities and plural terms shall include the singular.

The section headings used herein are for organizational purposes only and are not to be construed as limiting the desired subject matter in any way. In the event that any literature incorporated by reference contradicts any term defined in this specification, this specification controls. While the present teachings are described in conjunction with various embodiments, it is not intended that the present teachings be limited to such embodiments. On the contrary, the present teachings encompass various alternatives, modifications, and equivalents, as will be appreciated by those of skill in the art.

As used herein, a subject's “body weight” or “weight” is the mass of the subject. The body weight may be determined by a physician at a medical visit. In some embodiments, the body weight is determined on the day or one day prior to beginning dosing of trofinetide. In some embodiments, the subject has not fasted for the day prior to the body weight measurement.

As used herein, “treating” or “treatment” of Rett syndrome includes: (a) preventing the disease, i.e., causing the clinical symptoms of Rett syndrome not to develop or to develop more slowly in a subject that may be predisposed to Rett syndrome but does not yet experience or display one or more symptoms of the syndrome: (b) inhibiting the disease, i.e., arresting or reducing the development of the disease or its one or more symptoms of the syndrome, reducing and or preventing the progression of the disease or symptoms thereof, or relieving the disease, i.e., causing regression of the disease or its clinical symptoms.

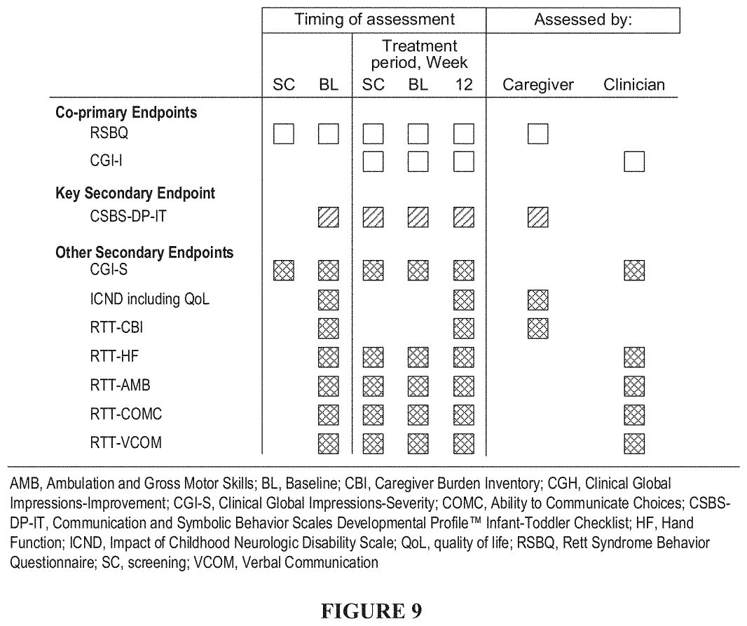

In some embodiments, the efficacy of the methods of treating is assessed by the Rett Syndrome Behavioral Questionnaire (RSBQ), the Rett Syndrome-Clinician Domain Specific Concerns (RTT-DSC), and/or the Clinical Global Impression Scale-Improvement scale (CGI-1) (Glaze et al., “Double-blind, randomized, placebo-controlled study of trofinetide in pediatric Rett syndrome,” Neurology 2019, 92(16), e1912-e1925). The RSGQ total score is a 45-item caregiver-completed rating scale assessing a wide range of neurobehavioral symptoms known to be impaired in RTT. The RSBQ is a well-validated checklist developed to assess behavioral and emotional characteristics of RTT. The RSBQ has been correlated with functioning and quality of life and has been characterized and validated across a range of ages and genetic variations in RTT. The scale includes 45 items, 39 of them grouped into eight subscales, whose ratings reflect the severity and frequency of symptoms. The eight subscales include general mood, breathing problems, hand behavior, face movements, body rocking/expressionless face, night-time behaviors, fear/anxiety, and walking/standing. The time frame for a placebo-controlled clinical trial may suitably be 12 weeks. In some embodiments, the efficacy of the methods of treating is assessed by the rating scale of Table 1 and may be combined with other measures. FIG. 9 provides an exemplary scheme for timing of assessments and assessors for exemplary endpoints for determination of efficacy.

The RTT-DSC is a clinician-completed visual analog scale (VAS) assessing the severity of concerns in hand use, ambulation, seizures, autonomic features, behavior, attentiveness, social interaction, and language/communication. Concerns are identified on an individual basis at baseline. Severity is scored for each concern by measuring the number of centimeters on a 10-cm VAS line and reported as a percentage of the line. A total VAS score is calculated as the sum of the scores for the eight concerns.

The CGI-I scale is a clinician-completed assessment of how much the individual's illness has improved or worsened relative to a baseline state, scored using a standardized rubric that is specific to the clinical features of RTT.

TABLE 1
RTT-specific clinical rating scales

| | Hand Function | Ambulation and Gross | Ability to Communicate | Verbal Communication |
|---|---|---|---|---|
| | (RTT-HF) | Motor Skills (RTT-AMB) | Choices (RTT-COMC) | (RTT-VCOM) |
| Area evaluated | Ability to use hands for | Ability to sit, stand, and | Ability to communicate | Ability to communicate |
| | functional purposes (eg, | ambulate (eg, walking, | choices or preferences, | verbally (eg, words and |
| | reaching for and | running, climbing stairs) | including nonverbal | phrases) |
| | grasping objects, self- | | means such as eye | |
| | feeding, drawing) | | contact or gestures | |
| 0 | Normal, no impairment | Normal, no impairment | Normal | Normal, no impairment |
| 1 | Can grip a writing | Stands and sits | Makes a forced choice | Uses phrases (not |
| | instrument effectively | independently AND | between 2 or more | exclusively “fixed |
| | and can draw a shape | Can move from sit to | drawings or symbolic | phrase”) or sentences |
| | (or has an equivalent | stand AND Walks | representations | May still feature |
| | fine motor skill) BUT | independently AND | | echolalia or |
| | Still has observable fine | Can climb up and down | | perseveration |
| | motor problems | stairs or run May still | | |
| | | have evidence of | | |
| | | dystonia, ataxia, or | | |
| | | dyspraxia | | |
| 2 | Can grasp objects AND | Stands and sits | Makes a forced choice | Uses many words (>20), |
| | Can selffeed AND Has | independently AND | between 4 photographs | which may include short |
| | a pincer grasp | Can move from sit to | of objects | “fixed phrases” that are |
| | | stand AND Walks | | used as if they were a |
| | | independently | | single word May feature |
| | | | | echolalia or |
| | | | | perseveration |
| 3 | Can grasp objects AND | Stands and sits | Makes a forced choice | Uses a few words (5- |
| | Can selffeed BUT Does | independently >30 | between 2 photographs | 20) AND The words are |
| | not have a pincer grasp | seconds AND Can walk | of objects | generally context- |
| | | independently but only | | appropriate |
| | | a short distance and | | |
| | | with reduced pace | | |
| 4 | Reaches for and can | Stands and sits | Makes a forced choice | Uses <5 words AND the |
| | grasp an object BUT | independently >30 | between 2 real-life | words are not |
| | Cannot self-feed (ie, the | seconds AND Walks | items (eg, food, toys, | necessarily context- |
| | hand functioning | with support across a | videos, shapes) | appropriate Often |
| | necessary to feed | range of distances | | occurs when stressed, |
| | oneself or drink by | | | anxious or |
| | oneself) | | | uncomfortable |
| 5 | Regularly reaches for | Stands AND Sits | Makes unforced choices | No words BUT Babbles |
| | objects AND Can hold | independently >30 | (eg, chooses food, a toy, | (makes consonant- |
| | object >2 seconds if | seconds OR Walks only | a video) | vowel combination |
| | placed in the hand BUT | a short distance (such as | | sounds) |
| | Cannot grasp objects | a few meters) with | | |
| | | support | | |
| 6 | Rarely or only | Sits independently >30 | Responds to name by | No words AND Makes |
| | occasionally reaches for | seconds OR Stands with | looking at speaker; | vocalizations (vowel- |
| | an object OR Can hold | support but unable to | Does not make choices | only sounds) BUT Does |
| | object >2 seconds if | take steps | | not babble (ie, does not |
| | placed in the hand BUT | | | make consonant-vowel |
| | Cannot grasp objects | | | combination sounds) |
| 7 | No hand use | Cannot sit without | No interactions or no | No words AND No |
| | | support AND Cannot | attempts to respond to | vocalizations (may |
| | | stand AND Cannot walk | requests even from | scream) |
| | | | caregivers; Does not | |
| | | | make choices | |

One aspect disclosed herein is a method of treating Rett syndrome, comprising administering trofinetide to a subject in need thereof in a daily amount of:
a) 4-12 g, such as 4-8 g, if the subject has a weight in the range of 8-12 kg;
b) 10-14 g if the subject has a weight in the range of 12-20 kg;
c) 14-18 g if the subject has a weight greater than 20 kg and less than or equal to 35 kg;
d) 18-22 g if the subject has a weight greater than 35 kg and less than or equal to 50 kg; or
e) 22-26 g if the subject has a weight greater than 50 kg.

Another aspect disclosed herein is a method of treating Rett syndrome, comprising administering trofinetide to a subject in need thereof in a daily amount of:
a) 4-12 g, such as 4-8 g, if the subject has a weight in the range of 8-12 kg;
b) 10-14 g if the subject has a weight in the range of 12-20 kg;
c) 14-18 g if the subject has a weight greater than 20 kg and less than or equal to 35 kg;
d) 18-22 g if the subject has a weight greater than 35 kg and less than or equal to 50 kg;
e) 22-26 g if the subject has a weight greater than 50 kg and less than or equal to 100 kg; or
f) 46-50 g if the subject has a weight greater than 100 kg.

Another aspect disclosed herein is a method of treating Rett syndrome, comprising administering trofinetide to a subject in need thereof in a daily amount of:
a) 10-14 g if the subject has a weight in the range of 12-20 kg;
b) 14-18 g if the subject has a weight greater than 20 kg and less than or equal to 35 kg;
c) 18-22 g if the subject has a weight greater than 35 kg and less than or equal to 50 kg.

Another aspect disclosed herein is a method of treating Rett syndrome, comprising administering trofinetide to a subject in need thereof in a daily amount of:
a) 8±x g or 6±x g, if the subject has a weight in the range of 8-12 kg;
b) 12±x g if the subject has a weight in the range of 12-20 kg;
c) 16±x g if the subject has a weight greater than 20 kg and less than or equal to 35 kg;
d) 20±x g if the subject has a weight greater than 35 kg and less than or equal to 50 kg; or
e) 24±x g if the subject has a weight greater than 50 kg
wherein x is 1, 2, 3, 4, 5, or 6, optionally wherein the daily amount is at least 3 g, further optionally wherein the daily amount is at least 4 g.

Another aspect disclosed herein is a method of treating Rett syndrome, comprising administering trofinetide to a subject in need thereof in a daily amount of:
a) 12±x g if the subject has a weight in the range of 12-20 kg;
b) 16±x g if the subject has a weight greater than 20 kg and less than or equal to 35 kg;
c) 20±x g if the subject has a weight greater than 35 kg and less than or equal to 50 kg; or
d) 24±x g if the subject has a weight greater than 50 kg,
wherein x is 1, 2, 3, 4, 5, or 6.

Another aspect disclosed herein is a method of treating Rett syndrome, comprising administering trofinetide to a subject in need thereof in a daily amount of:
a) 8±x g or 6±x g, if the subject has a weight in the range of 8-12 kg;
b) 12±x g if the subject has a weight in the range of 12-20 kg;
c) 16±x g if the subject has a weight greater than 20 kg and less than or equal to 35 kg;
d) 20±x g if the subject has a weight greater than 35 kg and less than or equal to 50 kg;
e) 24±x g if the subject has a weight greater than 50 kg and less than or equal to 100 kg; or
f) 48±x g if the subject has a weight greater than 100 kg,
wherein x is 1, 2, 3, 4, 5, or 6, optionally wherein the daily amount is at least 3 g, further optionally wherein the daily amount is at least 4 g.

Another aspect disclosed herein is a method of treating Rett syndrome, comprising administering trofinetide to a subject in need thereof in a daily amount of:
a) 12±x g if the subject has a weight in the range of 12-20 kg;
b) 16±x g if the subject has a weight greater than 20 kg and less than or equal to 35 kg; or
c) 20±x g if the subject has a weight greater than 35 kg and less than or equal to 50 kg.
wherein x is 1, 2, 3, 4, 5, or 6.

Another aspect disclosed herein is a method of treating Rett syndrome, comprising administering trofinetide to a subject in need thereof in a daily amount dₘₐₓ, wherein the subject has a weight w; d is less than or equal to dₘₐₓ and greater than or equal to dₘᵢₙ; and dₘᵢₙ and dₘₐₓ are calculated according to equations (I) and (II), respectively;

dₘᵢₙ=w/3750+6 g (I)

dₘₐₓ=w/3750+12 g (II).

In equations (I) and (II), the units of weight w can be any suitable weight unit, such as kilogram or pound, as long as the y-intercept values of 6 g in equation (T) and 12 g in equation (II) are adjusted accordingly. Exemplary values according to the method include:

| Weight (kg) | dₘᵢₙ (g) | dₘₐₓ (g) |
|---|---|---|
| 9 | 8.4 | 14.4 |
| 12 | 9.2 | 15.2 |
| 20 | 11⅓ | 17⅓ |
| 35 | 15⅓ | 21⅓ |
| 50 | 19⅓ | 25⅓ |
| 65 | 23⅓ | 29⅓ |
| 80 | 27⅓ | 33⅓ |

Another aspect disclosed herein is a method of treating Rett syndrome, comprising administering trofinetide to a subject in need thereof, wherein the trofinetide is administered twice daily in an amount sufficient to result in a 50^{th} percentile AUC₀₋₁₂ of at least 790 μg·h/mL. In some embodiments, the 50^{th} percentile AUC₀₋₁₂ is at least, or about, 888 μg·h/mL. In some embodiments, the trofinetide is administered twice daily in an amount sufficient to result in a 75^{th} percentile AUC₀₋₁₂ of at least 950 μg·h/mL.

Another aspect disclosed herein is a method of treating Rett syndrome, comprising administering trofinetide to a subject in need thereof, wherein the trofinetide is administered twice daily in an amount sufficient to result in an 80^{th} percentile AUC₀₋₁₂ of at least 800 μg·h/mL.

Another aspect disclosed herein is a method of treating Rett syndrome, comprising administering trofinetide to a subject in need thereof, wherein the trofinetide is administered twice daily in an amount sufficient to result in an 25^{th} percentile AUC₀₋₁₂ of at least 755 μg·h/mL.

Another aspect disclosed herein is a method of treating Rett syndrome, comprising administering trofinetide to a subject in need thereof, wherein the subject has a weight of less than 12 kg, such as 8-12 kg, and the trofinetide is administered in a daily amount of 4-12 g, such as 4-8 g.

Another aspect disclosed herein is a method of treating Rett syndrome, comprising administering trofinetide to a subject in need thereof, wherein the subject has a weight in the range of 12-20 kg and the trofinetide is administered in a daily amount of 10-14 g.

Another aspect disclosed herein is a method of treating Rett syndrome, comprising administering trofinetide to a subject in need thereof, wherein the subject has a weight greater than 20 kg and less than or equal to 35 kg and the trofinetide is administered in a daily amount of 14-18 g.

Another aspect disclosed herein is a method of treating Rett syndrome, comprising administering trofinetide to a subject in need thereof, wherein the subject has a weight greater than 35 kg and less than or equal to 50 kg and the trofinetide is administered in a daily amount of 18-22 g.

Another aspect disclosed herein is a method of treating Rett syndrome, comprising administering trofinetide to a subject in need thereof, wherein the subject has a weight greater than 50 kg and the trofinetide is administered in a daily amount of 22-26 g.

Another aspect disclosed herein is a method of treating Rett syndrome, comprising administering trofinetide to a subject in need thereof in a daily amount of:
a) about 12 g if the subject has a weight in the range of 12-20 kg;
b) about 16 g if the subject has a weight greater than 20 kg and less than or equal to 35 kg;
c) about 20 g if the subject has a weight greater than 35 kg and less than or equal to 50 kg; or
d) about 24 g if the subject has a weight greater than 50 kg.

Another aspect disclosed herein is a method of treating Rett syndrome, comprising administering trofinetide to a subject in need thereof in a daily amount of:
a) about 12 g if the subject has a weight in the range of 12-20 kg;
b) about 16 g if the subject has a weight greater than 20 kg and less than or equal to 35 kg; or
c) about 20 g if the subject has a weight greater than 35 kg and less than or equal to 50 kg.

Another aspect disclosed herein is a method of treating Rett syndrome, comprising administering trofinetide to a subject in need thereof in a daily amount of:
a) about 12 g if the subject has a weight in the range of 12-20 kg;
b) about 16 g if the subject has a weight greater than 20 kg and less than or equal to 35 kg;
c) about 20 g if the subject has a weight greater than 35 kg and less than or equal to 50 kg;
d) about 24 g if the subject has a weight greater than 50 kg and less than or equal to 100 kg; or
e) about 48 g if the subject has a weight greater than 100 kg.

In some embodiments, the subject has a weight in the range of 12-20 kg. In some embodiments, the subject has a weight greater than 20 kg and less than or equal to 35 kg. In some embodiments, wherein the subject has a weight greater than 35 kg and less than or equal to 50 kg. In some embodiments, the subject has a weight greater than 50 kg. In some embodiments, the subject has a weight of 50-80 kg, 50-75 kg, 50-70 kg, 50-65 kg, or 50-60 kg. In some embodiments, the weight of the subject is greater than 100 kg, optionally wherein the weight is less than or equal to 150 kg.

In some embodiments, the daily amount of trofinetide administered is about 6 g. In some embodiments, the daily amount of trofinetide administered is about 8 g. In some embodiments, the daily amount of trofinetide administered is a value in the range of 6-8 g. In some embodiments, the daily amount of trofinetide administered is about 12 g. In some embodiments, the daily amount is about 16 g. In some embodiments, the daily amount is about 20 g. In some embodiments, the daily amount is about 24 g. In some embodiments, the daily amount of trofinetide administered is about 48 g.

In some embodiments, the trofinetide is administered in a single dose per day. In some embodiments, the trofinetide is administered in multiple doses per day that sum to the daily amount, e.g., two, three, or four doses per day that sum to the daily amount. In some embodiments, the trofinetide is administered in two doses per day that sum to the daily amount. In some embodiments, the trofinetide is administered once in the morning and once in the evening. In some embodiments, there are at least eight hours between doses. In some embodiments, the subject does not eat for 1 hour before and for 1 hour after the trofinetide is administered. In some embodiments, doses are administered over a 10 minute period and/or are followed by providing water to the subject, e.g., in an amount of about 250 mL.

In some embodiments, the trofinetide is administered as a pharmaceutical composition comprising the trofinetide and a pharmaceutically acceptable carrier. Any of the well-known techniques and excipients may be used as suitable and as understood in the art, see for example, Remington: The Science and Practice of Pharmacy, Twenty-first Ed., (Pharmaceutical Press, 2005). In some embodiments, the pharmaceutically acceptable carrier is purified water. In some embodiments, the pharmaceutical composition comprises one or more excipients, such as a preservative, flavoring agent, coloring agent, time release-control agent, surfactant, diluent, buffering agent, stabilizing agent, antioxidant, antifoaming agent, or filler, or a mixture thereof. In some embodiments, the pharmaceutical composition is a solution, optionally an aqueous solution. In some embodiments, the pharmaceutical composition comprises one or more excipients selected from sweeteners (e.g., maltitol and/or sucralose), flavoring agents (e.g., strawberry flavor), preservatives (e.g., a methylparaben salt such as methylparaben sodium or a propylparaben salt such as propylparaben sodium, or a combination thereof), and coloring agents (e.g., FD&C Red #40). In some embodiments, the trofinetide has a concentration of less than 0.7 g/mL such as in the range of 0.05-0.6 g/mL, 0.1-0.4 g/mL, or 0.15-0.3 g/mL in the solution. In some embodiments, the trofinetide has a concentration of about 0.2 g/mL in the solution.

The pharmaceutical compositions described herein may be administered by any suitable mode of administration. In some embodiments, the trofinetide is administered orally. In some embodiments, the trofinetide is administered via gastrostomy tube or via a G-port of a gastrojejunal tube. In some embodiments, the trofinetide is administered intravenously.

In some embodiments, the subject is a mammal. In some embodiments, the subject is human. In some embodiments, the subject is a female. In some embodiments, the subject is 18 months to 60 years old. In some embodiments, the subject is 20 years old, or younger (e.g., 18 months to 20 years old). In some embodiments, the subject is 5-20 years old. In some embodiments, the subject is 5-10 years old, 11-15 years old, or 16-20 years old. In some embodiments, the subject is 2-5 years old. In some embodiments, the subject is older than 20 years old

Accordingly, the following embodiments are provided. Embodiment 1 is a method of treating Rett syndrome, comprising administering trofinetide to a subject in need thereof in a daily amount of:
a) 4-12 g, such as 4-8 g, if the subject has a weight in the range of 8-12 kg;
b) 10-14 g if the subject has a weight in the range of 12-20 kg;
c) 14-18 g if the subject has a weight greater than 20 kg and less than or equal to 35 kg;
d) 18-22 g if the subject has a weight greater than 35 kg and less than or equal to 50 kg; or
e) 22-26 g if the subject has a weight greater than 50 kg.

Embodiment 2 is a method of treating Rett syndrome, comprising administering trofinetide to a subject in need thereof in a daily amount of:
a) 8±x g or 6 r g, if the subject has a weight in the range of 8-12 kg;
b) 12±x g if the subject has a weight in the range of 12-20 kg;
c) 16±x g if the subject has a weight greater than 20 kg and less than or equal to 35 kg;
d) 20±x g if the subject has a weight greater than 35 kg and less than or equal to 50 kg; or
e) 24±x g if the subject has a weight greater than 50 kg,
wherein x is 1, 2, 3, 4, 5, or 6, optionally wherein the daily amount is at least 3 g, further optionally wherein the daily amount is at least 4 g.

Embodiment 3 is a method of treating Rett syndrome, comprising administering trofinetide to a subject in need thereof in a daily amount of:
a) 10-14 g if the subject has a weight in the range of 12-20 kg;
b) 14-18 g if the subject has a weight greater than 20 kg and less than or equal to 35 kg;
c) 18-22 g if the subject has a weight greater than 35 kg and less than or equal to 50 kg; or
d) 22-26 g if the subject has a weight greater than 50 kg.

Embodiment 4 is a method of treating Rett syndrome, comprising administering trofinetide to a subject in need thereof in a daily amount of:
a) 12±x g if the subject has a weight in the range of 12-20 kg;
b) 16±x g if the subject has a weight greater than 20 kg and less than or equal to 35 kg;
c) 20±x g if the subject has a weight greater than 35 kg and less than or equal to 50 kg; or
d) 24±x g if the subject has a weight greater than 50 kg,
wherein x is 1, 2, 3, 4, 5, or 6.

Embodiment 5 is a method of treating Rett syndrome, comprising administering trofinetide to a subject in need thereof in a daily amount of:
a) 4-12 g, such as 4-8 g, if the subject has a weight in the range of 8-12 kg;
b) 10-14 g if the subject has a weight in the range of 12-20 kg;
c) 14-18 g if the subject has a weight greater than 20 kg and less than or equal to 35 kg;
d) 18-22 g if the subject has a weight greater than 35 kg and less than or equal to 50 kg;
e) 22-26 g if the subject has a weight greater than 50 kg and less than or equal to 100 kg; or
f) 46-50 g if the subject has a weight greater than 100 kg.

Embodiment 6 is a method of treating Rett syndrome, comprising administering trofinetide to a subject in need thereof in a daily amount of:
a) 8±x g or 6±x g, if the subject has a weight in the range of 8-12 kg; b) 12±x g if the subject has a weight in the range of 12-20 kg;
c) 16±x g if the subject has a weight greater than 20 kg and less than or equal to 35 kg;
d) 20±x g if the subject has a weight greater than 35 kg and less than or equal to 50 kg;
e) 24±x g if the subject has a weight greater than 50 kg and less than or equal to 100 kg; or
f) 48±x g if the subject has a weight greater than 100 kg,
wherein x is 1, 2, 3, 4, 5, or 6, optionally wherein the daily amount is at least 3 g, further optionally wherein the daily amount is at least 4 g.

Embodiment 7 is a method of treating Rett syndrome, comprising administering trofinetide to a subject in need thereof in a daily amount of:
a) 10-14 g if the subject has a weight in the range of 12-20 kg;
b) 14-18 g if the subject has a weight greater than 20 kg and less than or equal to 35 kg; or
c) 18-22 g if the subject has a weight greater than 35 kg and less than or equal to 50 kg.

Embodiment 8 is a method of treating Rett syndrome, comprising administering trofinetide to a subject in need thereof in a daily amount of:
a) 12±x g if the subject has a weight in the range of 12-20 kg;
b) 16±x g if the subject has a weight greater than 20 kg and less than or equal to 35 kg; or
c) 20±x g if the subject has a weight greater than 35 kg and less than or equal to 50 kg;
wherein x is 1, 2, 3, 4, 5, or 6.

Embodiment 9 is a method of treating Rett syndrome, comprising administering trofinetide to a subject in need thereof in a daily amount d, wherein the subject has a weight w; d is less than or equal to d ax and greater than or equal to dₘᵢₙ; and dₘᵢₙ and dₘₐₓ are calculated according to equations (I) and (II), respectively:

dₘᵢₙ=w/3750+6 g (I)

dₘₐₓ=w/3750+12 g (II).

Embodiment 10 is a method of treating Rett syndrome, comprising administering trofinetide to a subject in need thereof, wherein the trofinetide is administered twice daily in an amount sufficient to result in an AUC₀₋₁₂ of at least 790 μg·h/mL.

Embodiment 11 is a method of treating Rett syndrome, comprising administering trofinetide to a subject in need thereof, wherein the trofinetide is administered twice daily in an amount sufficient to result in an AUC₀₋₁₂ of at least 800 μg·h/mL.

Embodiment 12 is a method of treating Rett syndrome, comprising administering trofinetide to a subject in need thereof, wherein the trofinetide is administered twice daily in an amount sufficient to result in an AUC₀₋₁₂ of at least 755 μg·h/mL.

Embodiment 13 is the method of any one of the preceding embodiments, wherein the subject has a weight in the range of 12-20 kg.

Embodiment 14 is a method of treating Rett syndrome, comprising administering trofinetide to a subject in need thereof, wherein the subject has a weight in the range of 12-20 kg and the trofinetide is administered in a daily amount of 10-14 g.

Embodiment 15 is the method of any one of the preceding embodiments, wherein the daily amount is about 12 g.

Embodiment 16 is the method of any one of embodiments 1-12, wherein the subject has a weight greater than 20 kg and less than or equal to 35 kg.

Embodiment 17 is a method of treating Rett syndrome, comprising administering trofinetide to a subject in need thereof, wherein the subject has a weight greater than 20 kg and less than or equal to 35 kg and the trofinetide is administered in a daily amount of 14-18 g.

Embodiment 18 is the method of embodiment 1-12, 16, or 17, wherein the daily amount is about 16 g.

Embodiment 19 is the method of any one of embodiments 1-12, wherein the subject has a weight greater than 35 kg and less than or equal to 50 kg.

Embodiment 20 is a method of treating Rett syndrome, comprising administering trofinetide to a subject in need thereof, wherein the subject has a weight greater than 35 kg and less than or equal to 50 kg and the trofinetide is administered in a daily amount of 18-22 g.

Embodiment 21 is the method of embodiment 1-12, 19, or 20, wherein the daily amount is about 20 g.

Embodiment 22 is the method of any one of embodiments 1-3 or 5-8, wherein the subject has a weight greater than 50 kg.

Embodiment 23 is a method of treating Rett syndrome, comprising administering trofinetide to a subject in need thereof, wherein the subject has a weight greater than 50 kg and the trofinetide is administered in a daily amount of 22-26 g, optionally wherein the weight is less than or equal to 100 kg.

Embodiment 24 is the method of embodiment 1-6, 9-12, 22, or 23, wherein the subject has a weight of 50-80 kg, 50-75 kg, 50-70 kg, 50-65 kg, or 50-60 kg.

Embodiment 25 is the method of any one of embodiments 1-6, 9-12, or 22-24, wherein the daily amount is about 24 g.

Embodiment 26 is the method of any one of embodiments 5, 6, or 9-12, wherein the weight of the subject is greater than 100 kg, optionally wherein the weight is less than or equal to 150 kg.

Embodiment 27 is the method of embodiment 26, wherein the daily amount is about 48 g.

Embodiment 28 is the method of any one of embodiments 1, 2, 5, or 6, wherein the subject has a weight in the range of 8-12 kg.

Embodiment 29 is the method of embodiment 28, wherein the daily amount is about 6 g or about 8 g, or a value in the range of 6-8 g.

Embodiment 30 is a method of treating Rett syndrome, comprising administering trofinetide to a subject in need thereof in a daily amount of:
a) about 12 g if the subject has a weight in the range of 12-20 kg;
b) about 16 g if the subject has a weight greater than 20 kg and less than or equal to 35 kg;
c) about 20 g if the subject has a weight greater than 35 kg and less than or equal to 50 kg; or
d) about 24 g if the subject has a weight greater than 50 kg.

Embodiment 31 is a method of treating Rett syndrome, comprising administering trofinetide to a subject in need thereof in a daily amount of:
a) about 12 g if the subject has a weight in the range of 12-20 kg;
b) about 16 g if the subject has a weight greater than 20 kg and less than or equal to 35 kg; or
c) about 20 g if the subject has a weight greater than 35 kg and less than or equal to 50 kg.

Embodiment 32 is a method of treating Rett syndrome, comprising administering trofinetide to a subject in need thereof in a daily amount of:
a) about 12 g if the subject has a weight in the range of 12-20 kg;
b) about 16 g if the subject has a weight greater than 20 kg and less than or equal to 35 kg;
c) about 20 g if the subject has a weight greater than 35 kg and less than or equal to 50 kg;
d) about 24 g if the subject has a weight greater than 50 kg and less than or equal to 100 kg; or
e) about 48 g if the subject has a weight greater than 100 kg.

Embodiment 33 is the method of any one of the preceding embodiments, wherein the trofinetide is administered twice daily and results in an AUC₀₋₁₂ of at least 790 μg·h/mL, such as in the range of 790-1000 μg·h/mL, optionally wherein the range is 790-950 μg·h/mL, 800-1000 μg·h/mL, or 800-950 μg·h/mL.

Embodiment 34 is the method of any one of the preceding embodiments, wherein the trofinetide is administered twice daily and results in an AUC₀₋₁₂ in the range of 790-1000 μg·h/mL, optionally wherein the range is 790-950 μg·h/mL, 800-1000 μg·h/mL, or 800-950 μg·h/mL.

Embodiment 35 is the method of any one of the preceding embodiments, wherein the trofinetide is administered twice daily in an amount and results in an AUC₀₋₁₂ in the range of 800-1300 μg·h/mL, optionally wherein the range is 800-1200 μg·h/mL, 1000-1300 μg·h/mL, or 1000-1200 μg·h/mL.

Embodiment 36 is the method of any one of the preceding embodiments, wherein the trofinetide is administered in multiple doses per day that sum to the daily amount.

Embodiment 37 is the method of any one of embodiments 1-9 or 13-32, wherein the trofinetide is administered in a single dose per day.

Embodiment 38 is the method of embodiment 37, wherein the trofinetide is administered in two doses per day that sum to the daily amount.

Embodiment 39 is the method of any one of the preceding embodiments, wherein the trofinetide is administered as a pharmaceutical composition comprising the trofinetide and a pharmaceutically acceptable carrier.

Embodiment 40 is the method of embodiment 39, wherein the pharmaceutical composition is a solution, optionally an aqueous solution.

Embodiment 41 is the method of embodiment 40, wherein the trofinetide has a concentration in the range of 0.05-0.7 g/mL, 0.05-0.6 g/mL, 0.1-0.4 g/mL, or 0.15-0.3 g/mL in the solution.

Embodiment 42 is the method of embodiment 41, wherein the trofinetide has a concentration of about 0.2 g/mL in the solution.

Embodiment 43 is the method of any one of the preceding embodiments, wherein the trofinetide is administered orally.

Embodiment 44 is the method of any one of the embodiments 1-42, wherein the trofinetide is administered via gastrostomy tube or via a G-port of a gastrojejunal tube.

Embodiment 45 is the method of any one of the preceding embodiments, wherein the subject is a mammal.

Embodiment 46 is the method of any one of the preceding embodiments, wherein the subject is human.

Embodiment 47 is the method of any one of the preceding embodiments, wherein the subject is a female.

Embodiment 48 is the method of any one of the preceding embodiments, wherein the subject is 18 months to 60 years old.

Embodiment 49 is the method of embodiment 48, wherein the subject is 5-20 years old, optionally wherein the subject is 5-10 years old, 11-15 years old, or 16-20 years old.

Embodiment 50 is the method of any one of the preceding embodiments, wherein the subject has a MECP2 mutation.

Embodiment 51 is the method of any one of the preceding embodiments, wherein the Rett syndrome is atypical or classic/typical Rett syndrome.

Embodiment 52 is the method of any one of the preceding embodiments, wherein the Rett syndrome is classic/typical Rett syndrome.

The following embodiments are also provided.

Embodiment 1. A method of treating Rett syndrome in a subject in need thereof, the method comprising administering a therapeutically effective amount of trofinetide to the subject in a daily amount of:
a) 4-10.0 g if the subject weighs between 8-11.9 kg;
b) 10.1-14.0 g if the subject weighs between 12.0-20.0 kg;
c) 14.1-18.0 g if the subject weighs between 20.1-35.0 kg;
d) 18.1-22.0 g if the subject weighs between 35.1-50.0 kg; or
e) 22.1-26 g if the subject weighs between 50.1-150 kg.

Embodiment II. The method of Embodiment I, wherein the subject weighs between 8-11.9 kg.

Embodiment III. The method of Embodiment II, wherein trofinetide is administered to the subject in a daily amount of 4 g, about 5 g, about 6 g, about 7 g, about 8 g, about 9 g, or 10 g, e.g. a daily amount of about 6 g, e.g. 6 g.

Embodiment IV. The method of Embodiment I, wherein the subject weighs between 12.0-20.0 kg.

Embodiment V. The method of Embodiment IV, wherein trofinetide is administered to the subject in a daily amount of 11 g, about 12 g, about 13 g, or 14 g, e.g., about 12 g, e.g., 12 g.

Embodiment VI. The method of Embodiment I, wherein the subject weighs between 20.1-35.0 kg.

Embodiment VII. The method of Embodiment VI, wherein trofinetide is administered to the subject in a daily amount of 15 g, about 16 g, about 17 g, or 18 g, e.g., about 16 g, e.g., 16 g.

Embodiment VIII. The method of Embodiment I, wherein the subject weighs between 35.1-50.0 kg.

Embodiment IX. The method of Embodiment VIII, wherein trofinetide is administered to the subject in a daily amount of 19 g, about 20 g, about 21 g, or 22 g, e.g. about 20 g, e.g, 20 g.

Embodiment X. The method of Embodiment I, wherein the subject weighs between 50.1-100 kg.

Embodiment XI. The method of Embodiment X, wherein trofinetide is administered to the subject in a daily amount of 23 g, about 24 g, about 25 g, or 26 g, e.g., about 24 g, e.g., 24 g.

Embodiment XII. The method of any one of Embodiments I-XI, wherein the administration of trofinetide results in an AUC_{0-12, ss} of about 755 μg·h/mL to about 1300 μg·h/mL, e.g., 755 μg·h/mL to 1300 μg·h/mL, 790 μg·h/mL to 1000 μg·h/mL, 790 μg·h/mL to 950 μg·h/mL, 800 μg·h/mL to 1000 μg·h/mL, 800 μg·h/mL to 950 μg·h/mL, 800 μg·h/mL to 1200 μg·h/mL, 1000 μg·h/mL to 1300 μg·h/mL, or 1000 μg·h/mL to 1200 μg·h/mL, in the subject.

Embodiment XIII. The method of any one of Embodiments I-XI, wherein the administration of trofinetide results in an AUC_{0-12, ss} of at least about 755 μg·h/mL, at least about 780 μg·h/mL, at least about 790 μg·h/mL, at least about 800 μg·h/mL, at least about 820 μg·h/mL, at least about 840 μg·h/mL, at least about 860 μg·h/mL, at least about 880 μg·h/mL, at least about 900 μg·h/mL, at least about 920 μg·h/mL, at least about 940 μg·h/mL, at least about 960 μg·h/mL, at least about 980 μg·h/mL, at least about 1000 μg·h/mL, at least about 1020 μg·h/mL, at least about 1040 μg·h/mL, at least about 1060 μg·h/mL, at least about 1080 μg·h/mL, at least about 1100 μg·h/mL, at least about 1120 μg·h/mL, at least about 1140 μg·h/mL, at least about 1160 μg·h/mL, at least about 1180 μg·h/mL, at least about 1200 μg·h/mL, at least about 1220 μg·h/mL, at least about 1240 μg·h/mL, at least about 1260 μg·h/mL, or at least about 1280 μg·h/mL, e.g., at least 755 μg·h/mL, at least 780 μg·h/mL, at least 790 μg·h/mL, at least 800 μg·h/mL, at least 820 μg·h/mL, at least 840 μg·h/mL, at least 860 μg·h/mL, at least 880 μg·h/mL, at least 900 μg·h/mL, at least 920 μg·h/mL, at least 940 μg·h/mL, at least 960 μg·h/mL, at least 980 μg·h/mL, at least 1000 μg·h/mL, at least 1020 μg·h/mL, at least 1040 μg·h/mL, at least 1060 μg·h/mL, at least 1080 μg·h/mL, at least 1100 μg·h/mL, at least 1120 μg·h/mL, at least 1140 μg·h/mL, at least 1160 μg·h/mL, at least 1180 μg·h/mL, at least 1200 μg·h/mL, at least 1220 μg·h/mL, at least 1240 μg·h/mL, at least 1260 μg·h/mL, or at least 1280 μg·h/mL, in the subject.

Embodiment XIV. The method of any one of Embodiments I-XIII, wherein the administration of trofinetide to the subject results in a C_{max, ss} of about 100 μg/mL to about 200 μg/mL, e.g., 100 μg/mL to 200 μg/mL, in the subject.

Embodiment XV. The method of any one of Embodiments I-XIII, wherein the administration of trofinetide to the subject results in a C_{max, ss} of at least about 100 μg/mL, at least about 110 μg/mL, at least about 120 μg/mL, at least about 130 μg/mL, at least about 140 μg/mL, at least about 150 μg/mL, at least about 160 μg/mL, at least about 170 μg/mL, at least about 180 μg/mL, at least about 190 μg/mL, or at least about 200 μg/mL, e.g., at least 100 μg/mL, at least 110 μg/mL, at least 120 μg/mL, at least 130 μg/mL, at least 140 μg/mL, at least 150 μg/mL, at least 160 μg/mL, at least 170 μg/mL, at least 180 μg/mL, at least 190 μg/mL, or at least 200 μg/mL, in the subject.

Embodiment XVI. The method of any one of Embodiments 1-XV, wherein trofinetide is administered to the subject in a single dose per day.

Embodiment XVII. The method of any one of Embodiments I-XV, wherein trofinetide is administered to the subject in multiple doses per day that sum to the daily amount.

Embodiment XVIII. The method of Embodiment XVII, wherein trofinetide is administered to the subject in two doses per day that sum to the daily amount.

Embodiment XIX. The method of any one of Embodiments I-XVIII, wherein trofinetide is administered as a pharmaceutical composition comprising trofinetide and a pharmaceutically acceptable carrier.

Embodiment XX. The method of Embodiment XIX, wherein the pharmaceutically acceptable carrier comprises water.

Embodiment XXI. The method of Embodiment XX, wherein the pharmaceutical composition is a solution.

Embodiment XXII. The method of Embodiment XXI, wherein the concentration of trofinetide is about 0.05 g/mL-0.7 g/mL in the solution.

Embodiment XXIII. The method of Embodiment XXII, wherein the concentration of trofinetide is about 0.2 g/mL in the solution.

Embodiment XXIV. The method of any one of Embodiments I-XXIII, wherein trofinetide is orally administered to the subject.

Embodiment XXV. The method of any one of Embodiments I-XXIV, wherein the subject is human.

Embodiment XXVI. The method of Embodiment XXV, wherein the subject is female.

Embodiment XXVII. The method of any one of Embodiments I-XXVI, wherein the subject is about 18 months to 20 years old, e.g., 2-5 years old.

Embodiment XXVIII. The method of any one of Embodiments I-XXVII, wherein the subject has a MECP2 mutation.

Embodiment XXIX. The method of any one of Embodiments I-XXVIII, wherein the Rett syndrome is atypical Rett syndrome.

Embodiment XXX. The method of any one of Embodiments I-XXVIII, wherein the Rett syndrome is classic/typical Rett syndrome.

Embodiment XXXI. Trofinetide for use in treating Rett syndrome in a subject, wherein trofinetide is to be administered in a daily amount of:
a) 4-10.0 g if the subject weighs between 8-11.9 kg;
b) 10.1-14.0 g if the subject weighs between 12.0-20.0 kg;
c) 14.1-18.0 g if the subject weighs between 20.1-35.0 kg;
d) 18.1-22.0 g if the subject weighs between 35.1-50.0 kg; or
e) 22.1-26 g if the subject weighs between 50.1-150 kg.

Embodiment XXXII. The trofinetide for use of Embodiment XXXI, wherein the subject weighs between 8-11.9 kg.

Embodiment XXXIII. The trofinetide for use of Embodiment XXXII, wherein trofinetide is to be administered to the subject in a daily amount of 4 g, about 5 g, about 6 g, about 7 g, about 8 g, about 9 g, or 10 g.

Embodiment XXXIV. The trofinetide for use of Embodiment XXXI, wherein the subject weighs between 12.0-20.0 kg.

Embodiment XXXV. The trofinetide for use of Embodiment XXXIV, wherein trofinetide is to be administered to the subject in a daily amount of 11 g, about 12 g, about 13 g, or 14 g.

Embodiment XXXVI. The trofinetide for use of Embodiment XXXI, wherein the subject weighs between 20.1-35.0 kg.

Embodiment XXXVII. The trofinetide for use of Embodiment XXXVI, wherein trofinetide is to be administered to the subject in a daily amount of 15 g, about 16 g, about 17 g, or 18 g.

Embodiment XXXVIII. The trofinetide for use of Embodiment XXXI, wherein the subject weighs between 35.1-50.0 kg.

Embodiment XXXIX. The trofinetide for use of Embodiment XXXVIII, wherein trofinetide is to be administered to the subject in a daily amount of 19 g, about 20 g, about 21 g, or 22 g.

Embodiment XL. The trofinetide for use of Embodiment XXXI, wherein the subject weighs between 50.1-100 kg.

Embodiment XLI. The trofinetide for use of Embodiment XL, wherein trofinetide is to be administered to the subject in a daily amount of 23 g, about 24 g, about 25 g, or 26 g.

Embodiment XLII. The trofinetide for use of any one of Embodiments XXXI-XLI, wherein the administration of trofinetide results in an AUC_{0-12, ss} of about 755 μg·h/mL to about 1300 μg·h/mL, e.g., about 800 μg·h/mL to about 1000 μg·h/mL, in the subject.

Embodiment XLIII. The trofinetide for use of any one of Embodiments XXXI-XLI, wherein the administration of trofinetide results in an AUC_{0-12, ss} of at least about 755 μg·h/mL, at least about 780 μg·h/mL, at least about 790 μg·h/mL, at least about 800 μg·h/mL, at least about 820 μg·h/mL, at least about 840 μg·h/mL, at least about 860 μg·h/mL, at least about 880 μg·h/mL, at least about 900 μg·h/mL, at least about 920 μg·h/mL, at least about 940 μg·h/mL, at least about 960 μg·h/mL, at least about 980 μg·h/mL, at least about 1000 μg·h/mL, at least about 1020 μg·h/mL, at least about 1040 μg·h/mL, at least about 1060 μg·h/mL, at least about 1080 μg·h/mL, at least about 1100 μg·h/mL, at least about 1120 μg·h/mL, at least about 1140 μg·h/mL, at least about 1160 μg·h/mL, at least about 1180 μg·h/mL, at least about 1200 μg·h/mL, at least about 1220 μg·h/mL, at least about 1240 μg·h/mL, at least about 1260 μg·h/mL, or at least about 1280 μg·h/mL in the subject.

Embodiment XLIV. The trofinetide for use of any one of Embodiments XXXI-XLIII, wherein the administration of trofinetide to the subject results in a C_{max, ss} of about 100 to about 200 μg/mL in the subject.

Embodiment XLV. The trofinetide for use of any one of Embodiments XXXI-XLIII, wherein the administration of trofinetide to the subject results in a C_{max, ss} of at least about 100 μg/mL, at least about 110 μg/mL, at least about 120 μg/mL, at least about 130 μg/mL, at least about 140 μg/mL, at least about 150 μg/mL, at least about 160 μg/mL, at least about 170 μg/mL, at least about 180 μg/mL, at least about 190 μg/mL, or at least about 200 μg/mL in the subject.

Embodiment XLVI. The trofinetide for use of any one of Embodiments XXXI-XLV, wherein trofinetide is to be administered to the subject in a single dose per day.

Embodiment XLVII. The trofinetide for use of any one of Embodiments XXXI-XLV, wherein trofinetide is to be administered to the subject in multiple doses per day that sum to the daily amount.

Embodiment XLVIII. The trofinetide for use of Embodiment XLVIII, wherein trofinetide is to be administered to the subject in two doses per day that sum to the daily amount.

Embodiment XLIX. The trofinetide for use of any one of Embodiments XXXI-XLVIII, wherein trofinetide is to be administered as a pharmaceutical composition comprising trofinetide and a pharmaceutically acceptable carrier.

Embodiment L. The trofinetide for use of Embodiment XLIX, wherein the pharmaceutically acceptable carrier comprises water.

Embodiment LI. The trofinetide for use of Embodiment L, wherein the pharmaceutical composition is a solution.

Embodiment LII. The trofinetide for use of Embodiment LI, wherein the concentration of trofinetide is about 0.05 g/mL-0.7 g/mL in the solution.

Embodiment LIII. The trofinetide for use of Embodiment LII, wherein the concentration of trofinetide is about 0.2 g/mL in the solution.

Embodiment LIV. The trofinetide for use of any one of Embodiments XXXI-LIII, wherein trofinetide is to be orally administered to the subject.

Embodiment LV. The trofinetide for use of any one of Embodiments XXXI-LIV, wherein the subject is human.

Embodiment LVI. The trofinetide for use of Embodiment LV, wherein the subject is female.

Embodiment LVII. The trofinetide for use of any one of Embodiments XXXI-LVI, wherein the subject is about 18 months to 20 years old, e.g., 2-5 years old.

Embodiment LVIII. The trofinetide for use of any one of Embodiments XXXI-LVII, wherein the subject has a MECP2 mutation.

Embodiment LIX. The trofinetide for use of any one of Embodiments XXXI-LVIII, wherein the Rett syndrome is atypical Rett syndrome.

Embodiment LX. The trofinetide for use of any one of Embodiments XXXI-LVIII, wherein the Rett syndrome is classic/typical Rett syndrome.

Embodiment LXI. Use of Trofinetide for the manufacture of a medicament for treating Rett syndrome in a subject, wherein trofinetide is to be administered in a daily amount of:
a) 4-10.0 g if the subject weighs between 8-11.9 kg;
b) 10.1-14.0 g if the subject weighs between 12.0-20.0 kg;
c) 14.1-18.0 g if the subject weighs between 20.1-35.0 kg;
d) 18.1-22.0 g if the subject weighs between 35.1-50.0 kg; or
e) 22.1-26 g if the subject weighs between 50.1-150 kg.

Embodiment LXII. The use of Embodiment LXI, wherein the subject weighs between 8-11.9 kg.

Embodiment LXIII. The use of Embodiment LXII, wherein trofinetide is to be administered to the subject in a daily amount of 4 g, about 5 g, about 6 g, about 7 g, about 8 g, about 9 g, or 10 g.

Embodiment LXIV. The use of Embodiment LXI, wherein the subject weighs between 12.0-20.0 kg.

Embodiment LXV. The use of Embodiment LXIV, wherein trofinetide is to be administered to the subject in a daily amount of 11 g, about 12 g, about 13 g, or 14 g.

Embodiment LXVI. The use of Embodiment LXI, wherein the subject weighs between 20.1-35.0 kg.

Embodiment LXVII. The use of Embodiment LXVI, wherein trofinetide is to be administered to the subject in a daily amount of 15 g, about 16 g, about 17 g, or 18 g.

Embodiment LXVIII. The use of Embodiment LXI, wherein the subject weighs between 35.1-50.0 kg.

Embodiment LXIX. The use of Embodiment LXVIII, wherein trofinetide is to be administered to the subject in a daily amount of 19 g, about 20 g, about 21 g, or 22 g.

Embodiment LXX. The use of Embodiment LXI, wherein the subject weighs between 50.1-100 kg.

Embodiment LXXI. The use of Embodiment LXX, wherein trofinetide is to be administered to the subject in a daily amount of 23 g, about 24 g, about 25 g, or 26 g.

Embodiment LXXII. The use of any one of Embodiments LXI-LXXI, wherein the administration of trofinetide results in an AUC_{0-12, ss} of about 755 μg·h/mL to about 1300 μg·h/mL, e.g., about 800 μg·h/mL to about 1000 μg·h/mL, in the subject.

Embodiment LXXIII. The use of any one of Embodiments LXI-LXXI, wherein the administration of trofinetide results in an AUC_{0-12, ss} of at least about 755 μg·h/mL, at least about 780 μg·h/mL, at least about 790 μg·h/mL, at least about 800 μg·h/mL, at least about 820 μg·h/mL, at least about 840 μg·h/mL, at least about 860 μg·h/mL, at least about 880 μg·h/mL, at least about 900 μg·h/mL, at least about 920 μg·h/mL, at least about 940 μg·h/mL, at least about 960 μg·h/mL, at least about 980 μg·h/mL, at least about 1000 μg·h/mL, at least about 1020 μg·h/mL, at least about 1040 μg·h/mL, at least about 1060 μg·h/mL, at least about 1080 ugh/mL, at least about 1100 μg·h/mL, at least about 1120 μg·h/mL, at least about 1140 μg·h/mL, at least about 1160 μg·h/mL, at least about 1180 μg·h/mL, at least about 1200 μg·h/mL, at least about 1220 μg·h/mL, at least about 1240 μg·h/mL, at least about 1260 μg·h/mL, or at least about 1280 μg·h/mL in the subject.

Embodiment LXXIV. The use of any one of Embodiments LXI-LXXIII, wherein the administration of trofinetide to the subject results in a C_{max, ss} of about 100 to about 200 μg/mL in the subject.

Embodiment LXXV. The use of Embodiments LXI-LXXIII, wherein the administration of trofinetide to the subject results in a C_{max, ss} of at least about 100 μg/mL, at least about 110 μg/mL, at least about 120 μg/mL, at least about 130 μg/mL, at least about 140 μg/mL, at least about 150 μg/mL, at least about 160 μg/mL, at least about 170 μg/mL, at least about 180 μg/mL, at least about 190 μg/mL, or at least about 200 μg/mL in the subject.

Embodiment LXXVI. The trofinetide for use of any one of Embodiments LXI-LXXV, wherein trofinetide is to be administered to the subject in a single dose per day.

Embodiment LXXVII. The use of any one of Embodiments LXI-LXXV, wherein trofinetide is to be administered to the subject in multiple doses per day that sum to the daily amount.

Embodiment LXXVIII. The use of Embodiment LXXVII, wherein trofinetide is to be administered to the subject in two doses per day that sum to the daily amount.

Embodiment LXXIX. The use of any one of Embodiments LXI-LXXVIII, wherein trofinetide is to be administered as a pharmaceutical composition comprising trofinetide and a pharmaceutically acceptable carrier.

Embodiment LXXX. The use of Embodiment LXXIX, wherein the pharmaceutically acceptable carrier comprises water.

Embodiment LXXXI. The use of Embodiment LXXIX, wherein the pharmaceutical composition is a solution.

Embodiment LXXXII. The use of Embodiment LXXXI, wherein the concentration of trofinetide is about 0.05 g/mL-0.7 g/mL in the solution.

Embodiment LXXXIII. The use of Embodiment LXXXII, wherein the concentration of trofinetide is about 0.2 g/mL in the solution.

Embodiment LXXXIV. The use of any one of Embodiments LXI-LXXXIII, wherein trofinetide is to be orally administered to the subject.

Embodiment LXXXV. The use of any one of Embodiments LXI-LXXXIV, wherein the subject is human.

Embodiment LXXXVI. The use of Embodiment LXXXV, wherein the subject is female.

Embodiment LXXXVII. The use of any one of Embodiments LXI-LXXXVI, wherein the subject is about 18 months to about 20 years old, e.g., 2-5 years old.

Embodiment LXXXVIII. The use of any one of Embodiments LXI-LXXXVII, wherein the subject has a MECP2 mutation.

Embodiment LXXXIX. The use of any one of Embodiments LXI-LXXXVIII, wherein the Rett syndrome is atypical Rett syndrome.

Embodiment XC. The use of any one of Embodiments LXI-LXXXVIII, wherein the Rett syndrome is classic/typical Rett syndrome.

Embodiment XCI. A method of treating Rett syndrome in a human subject in need thereof, the method comprising administering a therapeutically effective amount of trofinetide to the subject in a daily amount of about 500 mg/kg to about 1,250 mg/kg, wherein the subject is about 15 months to about 6 years old.

Embodiment XCII. The method of Embodiment XCI, comprising administering a daily amount of about 500 mg/kg to the subject.

Embodiment XCIII. The method of Embodiment XCI, comprising administering a daily amount of about 600 mg/kg to the subject.

Embodiment XCIV. The method of Embodiment XCI, comprising administering a daily amount of about 700 mg/kg to the subject.

Embodiment XCV. The method of Embodiment XCI, comprising administering a daily amount of about 800 mg/kg to the subject.

Embodiment XCVI. The method of Embodiment XCI, comprising administering a daily amount of about 900 mg/kg to the subject.

Embodiment XCVII. The method of Embodiment XCI, comprising administering a daily amount of about 1,000 mg/kg to the subject.

Embodiment XCVIII. The method of Embodiment XCI, comprising administering a daily amount of about 1,100 mg/kg to the subject.

Embodiment XCIX. The method of Embodiment XCI, comprising administering a daily amount of about 1,200 mg/kg to the subject.

Embodiment C. The method of any one of Embodiments XCI-XCIX, wherein the administration of trofinetide results in an AUC_{0-12, ss} of about 755 μg·h/mL to about 1300 μg·h/mL, e.g., about 800 μg·h/mL to about 1000 μg·h/mL, in the subject.

Embodiment CI. The method of any one of Embodiments XCI-XCIX, wherein the administration of trofinetide results in an AUC_{0-12, ss} of at least about 755 μg·h/mL, at least about 780 μg·h/mL, at least about 790 μg·h/mL, at least about 800 μg·h/mL, at least about 820 μg·h/mL, at least about 840 μg·h/mL, at least about 860 μg·h/mL, at least about 880 μg·h/mL, at least about 900 μg·h/mL, at least about 920 μg·h/mL, at least about 940 μg·h/mL, at least about 960 μg·h/mL, at least about 980 μg·h/mL, at least about 1000 μg·h/mL, at least about 1020 μg·h/mL, at least about 1040 μg·h/mL, at least about 1060 μg·h/mL, at least about 1080 μg·h/mL, at least about 1100 μg·h/mL, at least about 1120 μg·h/mL, at least about 1140 μg·h/mL, at least about 1160 μg·h/mL, at least about 1180 μg·h/mL, at least about 1200 μg·h/mL, at least about 1220 μg·h/mL, at least about 1240 μg·h/mL, at least about 1260 μg·h/mL, or at least about 1280 μg·h/mL in the subject.

Embodiment CII. The method of any one of Embodiments XCI-CI, wherein the administration of trofinetide to the subject results in a C_{max, ss} of about 100 to about 200 μg/mL in the subject.

Embodiment CIII. The method of any one of Embodiments XCI-CI, wherein the administration of trofinetide to the subject results in a C_{max, ss} of at least about 100 μg/mL, at least about 110 μg/mL, at least about 120 μg/mL, at least about 130 μg/mL, at least about 140 μg/mL, at least about 150 μg/mL, at least about 160 μg/mL, at least about 170 μg/mL, at least about 180 μg/mL, at least about 190 μg/mL, or at least about 200 μg/mL in the subject.

Embodiment CIV. The method of any one of Embodiments XCI-CIII, wherein trofinetide is administered to the subject in a single dose per day.

Embodiment CV. The method of any one of Embodiments XCI-CIV, wherein trofinetide is administered to the subject in multiple doses per day that sum to the daily amount.

Embodiment CVI. The method of Embodiment CV, wherein trofinetide is administered to the subject in two doses per day that sum to the daily amount.

Embodiment CVII. The method of any one of Embodiments XCI-CVI, wherein trofinetide is administered as a pharmaceutical composition comprising trofinetide and a pharmaceutically acceptable carrier.

Embodiment CVIII. The method of Embodiment CVII, wherein the pharmaceutically acceptable carrier comprises water.

Embodiment CIX. The method of Embodiment CVIII, wherein the pharmaceutical composition is a solution.

Embodiment CX. The method of Embodiment CIX, wherein the concentration of trofinetide is about 0.05 g/mL-0.7 g/mL in the solution.

Embodiment CXI. The method of Embodiment CX, wherein the concentration of trofinetide is about 0.2 g/mL in the solution.

Embodiment CXII. The method of any one of Embodiments XCI-CXI, wherein trofinetide is orally administered to the subject.

Embodiment CXIII. The method of any one of Embodiments XCI-CXII, wherein the subject is female.

Embodiment CXIV. The method of any one of claims Embodiments XCI-CXIII, wherein the subject has a MECP2 mutation.

Embodiment CXV. The method of any one of Embodiments XCI-CXIV, wherein the Rett syndrome is atypical Rett syndrome.

Embodiment CXVI. The method of any one of Embodiments XCI-CXIV, wherein the Rett syndrome is classic/typical Rett syndrome.

Embodiment CXVII. The method of any one of Embodiments XCI-CXVI, wherein the subject is about 2 years old.

Embodiment CXVIII. The method of any one of Embodiments XCI-CXVI, wherein the subject is about 3 years old.

Embodiment CXIX. The method of any one of Embodiments XCI-CXVI, wherein the subject is about 4 years old.

Embodiment CXX. The method of any one of Embodiments XCI-CXVI, wherein the subject is about 5 years old.

Embodiment CXXI. Trofinetide for use in treating Rett syndrome in a human subject, wherein trofinetide is to be administered in a daily amount of about 500 mg/kg to about 1,250 mg/kg, wherein the subject is about 15 months to about 6 years old.

Embodiment CXXII. The trofinetide for use of Embodiment CXXI, comprising administering a daily amount of about 500 mg/kg to the subject.

Embodiment CXXIII. The trofinetide for use of Embodiment CXXI, comprising administering a daily amount of about 600 mg/kg to the subject.

Embodiment CXXIV. The trofinetide for use of Embodiment CXXI, comprising administering a daily amount of about 700 mg/kg to the subject.

Embodiment CXXV. The trofinetide for use of Embodiment CXXI, comprising administering a daily amount of about 800 mg/kg to the subject.

Embodiment CXXVI. The trofinetide for use hod of Embodiment CXXI, comprising administering a daily amount of about 900 mg/kg to the subject.

Embodiment CXXVII. The trofinetide for use of Embodiment CXXI, comprising administering a daily amount of about 1,000 mg/kg to the subject.

Embodiment CXXVIII. The trofinetide for use of Embodiment CXXI, comprising administering a daily amount of about 1,100 mg/kg to the subject.

Embodiment CXXIX. The trofinetide for use of Embodiment CXXI, comprising administering a daily amount of about 1,200 mg/kg to the subject.

Embodiment CXXX. The trofinetide for use of any one of Embodiments CXXI-CXXIX, wherein the administration of trofinetide results in an AUC_{0-12, ss} of about 755 μg·h/mL to about 1300 μg·h/mL, e.g., about 800 μg·h/mL to about 1000 μg·h/mL, in the subject.

Embodiment CXXXI. The trofinetide for use of any one of Embodiments CXXI-CXXIX, wherein the administration of trofinetide results in an AUC_{0-12, ss} of at least about 755 μg·h/mL, at least about 780 μg·h/mL, at least about 790 μg·h/mL, at least about 800 μg·h/mL, at least about 820 μg·h/mL, at least about 840 μg·h/mL, at least about 860 μg·h/mL, at least about 880 μg·h/mL, at least about 900 μg·h/mL, at least about 920 μg·h/mL, at least about 940 μg·h/mL, at least about 960 μg·h/mL, at least about 980 μg·h/mL, at least about 1000 μg·h/mL, at least about 1020 μg·h/mL, at least about 1040 μg·h/mL, at least about 1060 μg·h/mL, at least about 1080 μg·h/mL, at least about 1100 μg·h/mL, at least about 1120 μg·h/mL, at least about 1140 μg·h/mL, at least about 1160 μg·h/mL, at least about 1180 μg·h/mL, at least about 1200 μg·h/mL, at least about 1220 μg·h/mL, at least about 1240 μg·h/mL, at least about 1260 μg·h/mL, or at least about 1280 μg·h/mL in the subject.

Embodiment CXXXII. The trofinetide for use of any one of Embodiments CXXI-CXXXI, wherein the administration of trofinetide to the subject results in a C_{max, ss} of about 100 to about 200 μg/mL in the subject.

Embodiment CXXXIII. The trofinetide for use of any one of Embodiments CXXI-CXXXI, wherein the administration of trofinetide to the subject results in a C_{max, ss} at least about 100 μg/mL, at least about 110 μg/mL, at least about 120 μg/mL, at least about 130 μg/mL, at least about 140 μg/mL, at least about 150 μg/mL, at least about 160 μg/mL, at least about 170 μg/mL, at least about 180 μg/mL, at least about 190 μg/mL, or at least about 200 μg/mL in the subject.

Embodiment CXXXIV. The trofinetide for use of any one of Embodiments CXXI-CXXXIII, wherein trofinetide is administered to the subject in a single dose per day.

Embodiment CXXXV. The trofinetide for use of any one of Embodiments CXXI-CXXXIV, wherein trofinetide is administered to the subject in multiple doses per day that sum to the daily amount.

Embodiment CXXXVI. The trofinetide for use of Embodiment CXXXV, wherein trofinetide is administered to the subject in two doses per day that sum to the daily amount.

Embodiment CXXXVII. The trofinetide for use of any one of Embodiments CXXI-CXXXVI, wherein trofinetide is administered as a pharmaceutical composition comprising trofinetide and a pharmaceutically acceptable carrier.

Embodiment CXXXVIII. The trofinetide for use of Embodiment CXXXVII, wherein the pharmaceutically acceptable carrier comprises water.

Embodiment CXXXIX. The trofinetide for use of Embodiment CXXXVIII, wherein the pharmaceutical composition is a solution.

Embodiment CXL. The trofinetide for use of Embodiment CXXXIX, wherein the concentration of trofinetide is about 0.05 g/mL-0.7 g/mL in the solution.

Embodiment CXLI. The trofinetide for use of Embodiment CXL, wherein the concentration of trofinetide is about 0.2 g/mL in the solution.

Embodiment CXLII. The trofinetide for use of any one of Embodiments CXXI-CXLI, wherein trofinetide is orally administered to the subject.

Embodiment CXLIII. The trofinetide for use of any one of Embodiments CXXI-CXLII, wherein the subject is female.

Embodiment CXLIV. The trofinetide for use of any one of claims Embodiments CXXI-CXLIII, wherein the subject has a MECP2 mutation.

Embodiment CXLV. The trofinetide for use of any one of Embodiments CXXI-CXLIV, wherein the Rett syndrome is atypical Rett syndrome.

Embodiment CXLVI. The trofinetide for use of any one of Embodiments CXXI-CXLIV, wherein the Rett syndrome is classic/typical Rett syndrome.

Embodiment CXLVII. The trofinetide for use of any one of Embodiments CXXI-CXLVI, wherein the subject is about 2 years old.

Embodiment CXLVIII. The trofinetide for use of any one of Embodiments CXXI-CXLVI, wherein the subject is about 3 years old.

Embodiment CXLIX. The trofinetide for use of any one of Embodiments CXXI-CXLVI, wherein the subject is about 4 years old.

Embodiment CL. The trofinetide for use of any one of Embodiments CXXL-CXLVI, wherein the subject is about 5 years old.

Embodiment CLI. Use of Trofinetide for the manufacture of a medicament for treating Rett syndrome in a human subject, wherein trofinetide is to be administered in a daily amount of about 500 mg/kg to about 1,250 mg/kg, wherein the subject is about 15 months to about 6 years old.

Embodiment CLII. The use of Embodiment CLI, comprising administering a daily amount of about 500 mg/kg to the subject.

Embodiment CLIII. The use of Embodiment CLI, comprising administering a daily amount of about 600 mg/kg to the subject.

Embodiment CLIV. The use of Embodiment CLI, comprising administering a daily amount of about 700 mg/kg to the subject.

Embodiment CLV. The use of Embodiment CLI, comprising administering a daily amount of about 800 mg/kg to the subject.

Embodiment CLVI. The use hod of Embodiment CLI, comprising administering a daily amount of about 900 mg/kg to the subject.

Embodiment CLVII. The use of Embodiment CLI, comprising administering a daily amount of about 1,000 mg/kg to the subject.

Embodiment CLVIII. The trofinetide for use of Embodiment CLI, comprising administering a daily amount of about 1,100 mg/kg to the subject.

Embodiment CLIX. The use of Embodiment CLI, comprising administering a daily amount of about 1,200 mg/kg to the subject.

Embodiment CLX. The use of any one of Embodiments CLI-CLIX, wherein the administration of trofinetide results in an AUC_{0-12, ss} of about 755 μg·h/mL to about 1300 μg·h/mL, e.g., about 800 μg·h/mL to about 1000 μg·h/mL, in the subject.

Embodiment CLXI. The use of any one of Embodiments CLI-CLIX, wherein the administration of trofinetide results in an AUC_{0-12, ss} of at least about 755 μg·h/mL, at least about 780 μg·h/mL, at least about 790 μg·h/mL, at least about 800 μg·h/mL, at least about 820 μg·h/mL, at least about 840 μg·h/mL, at least about 860 μg·h/mL, at least about 880 μg·h/mL, at least about 900 μg·h/mL, at least about 920 μg·h/mL, at least about 940 μg·h/mL, at least about 960 μg·h/mL, at least about 980 μg·h/mL, at least about 1000 μg·h/mL, at least about 1020 μg·h/mL, at least about 1040 μg·h/mL, at least about 1060 μg·h/mL, at least about 1080 μg·h/mL, at least about 1100 μg·h/mL, at least about 1120 μg·h/mL, at least about 1140 μg·h/mL, at least about 1160 μg·h/mL, at least about 1180 μg·h/mL, at least about 1200 μg·h/mL, at least about 1220 μg·h/mL, at least about 1240 μg·h/mL, at least about 1260 μg·h/mL, or at least about 1280 μg·h/mL in the subject.

Embodiment CLXII. The use of any one of Embodiments CLI-CLXI, wherein the administration of trofinetide to the subject results in a C_{max, ss} of about 100 to about 200 μg/mL in the subject.

Embodiment CLXIII. The use of any one of Embodiments CLI-CLXI, wherein the administration of trofinetide to the subject results in a C_{max, ss} of at least about 100 μg/mL, at least about 110 μg/mL, at least about 120 μg/mL, at least about 130 μg/mL, at least about 140 μg/mL, at least about 150 μg/mL, at least about 160 μg/mL, at least about 170 μg/mL, at least about 180 μg/mL, at least about 190 μg/mL, or at least about 200 μg/mL in the subject.

Embodiment CLXIV. The use of any one of Embodiments CLI-CLXIII, wherein trofinetide is administered to the subject in a single dose per day.

Embodiment CLXV. The use of any one of Embodiments CLI-CLXIV, wherein trofinetide is administered to the subject in multiple doses per day that sum to the daily amount.

Embodiment CLXVI. The use of Embodiment CLXV, wherein trofinetide is administered to the subject in two doses per day that sum to the daily amount.

Embodiment CLXVII. The use of any one of Embodiments CLI-CLXVI, wherein trofinetide is administered as a pharmaceutical composition comprising trofinetide and a pharmaceutically acceptable carrier.

Embodiment CLXVIII. The use of Embodiment CLXVII, wherein the pharmaceutically acceptable carrier comprises water.

Embodiment CLXIX. The use of Embodiment CLXVIII, wherein the pharmaceutical composition is a solution.

Embodiment CLXX. The use of Embodiment CLXIX, wherein the concentration of trofinetide is about 0.05 g/mL-0.7 g/mL in the solution.

Embodiment CLXXI. The use of Embodiment CLXX, wherein the concentration of trofinetide is about 0.2 g/mL in the solution.

Embodiment CLXXII. The use of any one of Embodiments CLI-CLXXLI, wherein trofinetide is orally administered to the subject.

Embodiment CLXXIII. The use of any one of Embodiments CLI-CLXXII, wherein the subject is female.

Embodiment CLXXIV. The use of any one of claims Embodiments CLI-CLXXIII, wherein the subject has a MECP2 mutation.

Embodiment CLXXV. The use of any one of Embodiments CLI-CLXXIV, wherein the Rett syndrome is atypical Rett syndrome.

Embodiment CLXXVI. The use of any one of Embodiments CLI-CLXXIV, wherein the Rett syndrome is classic/typical Rett syndrome.

Embodiment CLXXVII. The use of any one of Embodiments CLI-CLXXVI, wherein the subject is about 2 years old.

Embodiment CLXXVIII. The use of any one of Embodiments CLI-CLXXVI, wherein the subject is about 3 years old.

Embodiment CLXXIX. The use of any one of Embodiments CLI-CLXXVI, wherein the subject is about 4 years old.

Embodiment CLXXX. The use of any one of Embodiments CLI-CLXXVI, wherein the subject is about 5 years old.

Embodiment CLXXXI. The method of any one of Embodiments I-XI or XIV-XXX, wherein the administration of trofinetide results in an AUC_{0-12, ss} of at least 755 μg·h/mL in the subject, such as 755 μg·h/mL to 1300 μg·h/mL, such as 780 μg·h/mL to 1300 μg·h/mL, such as 790 μg·h/mL to 1300 μg·h/mL, such as 800 μg·h/mL to 1300 μg·h/mL, such as 820 μg·h/mL to 1300 μg·h/mL, such as 840 μg·h/mL to 1300 μg·h/mL, such as 860 μg·h/mL to 1300 μg·h/mL, such as 880 μg·h/mL to 1300 μg·h/mL, such as 900 μg·h/mL to 1300 μg·h/mL, such as 920 μg·h/mL to 1300 μg·h/mL, such as 940 μg·h/mL to 1300 μg·h/mL, such as 960 μg·h/mL to 1300 μg·h/mL, such as 980 μg·h/mL to 1300 μg·h/mL, such as 1000 μg·h/mL to 1300 μg·h/mL, such as 1020 μg·h/mL to 1300 μg·h/mL, such as 1040 μg·h/mL to 1300 μg·h/mL, such as 1060 μg·h/mL to 1300 μg·h/mL, such as 1080 μg·h/mL to 1300 μg·h/mL, such as 1100 μg·h/mL to 1300 μg·h/mL, such as 1120 μg·h/mL to 1300 μg·h/mL, such as 1140 μg·h/mL to 1300 μg·h/mL, such as 1160 μg·h/mL to 1300 μg·h/mL, such as 1180 μg·h/mL to 1300 μg·h/mL, such as 1200 μg·h/mL to 1300 μg·h/mL, such as 1220 μg·h/mL to 1300 μg·h/mL, such as 1240 μg·h/mL to 1300 μg·h/mL, such as 1260 μg·h/mL to 1300 μg·h/mL, or such as 1280 μg·h/mL to 1300 μg·h/mL, in the subject. Embodiment CLXXXII. The method of Embodiment CLXXXI, wherein the administration of trofinetide results in an AUC_{0-12, ss} of 755 μg·h/mL to 1300 μg·h/mL in the subject.

Embodiment CLXXXIII. The method of Embodiment CLXXXII, wherein the administration of trofinetide results in an AUC_{0-12, ss} of 780 μg·h/mL to 1300 μg·h/mL in the subject.

Embodiment CLXXXIV. The method of Embodiment CLXXXIII, wherein the administration of trofinetide results in an AUC_{0-12, ss} of 790 μg·h/mL to 1300 μg·h/mL in the subject.

Embodiment CLXXXV. The trofinetide for use of any one of Embodiments XXXI-XLI or XLIV-LX, wherein the administration of trofinetide results in an AUC_{0-12, ss} of at least 755 μg·h/mL in the subject, such as 755 μg·h/mL to 1300 μg·h/mL, such as 780 μg·h/mL to 1300 μg·h/mL, such as 790 μg·h/mL to 1300 μg·h/mL, such as 800 μg·h/mL to 1300 μg·h/mL, such as 820 μg·h/mL to 1300 μg·h/mL, such as 840 μg·h/mL to 1300 μg·h/mL, such as 860 μg·h/mL to 1300 μg·h/mL, such as 880 μg·h/mL to 1300 μg·h/mL, such as 900 μg·h/mL to 1300 μg·h/mL, such as 920 μg·h/mL to 1300 μg·h/mL, such as 940 μg·h/mL to 1300 μg·h/mL, such as 960 μg·h/mL to 1300 μg·h/mL, such as 980 μg·h/mL to 1300 μg·h/mL, such as 1000 μg·h/mL to 1300 μg·h/mL, such as 1020 μg·h/mL to 1300 μg·h/mL, such as 1040 μg·h/mL to 1300 μg·h/mL, such as 1060 μg·h/mL to 1300 μg·h/mL, such as 1080 μg·h/mL to 1300 μg·h/mL, such as 1100 μg·h/mL to 1300 μg·h/mL, such as 1120 μg·h/mL to 1300 μg·h/mL, such as 1140 μg·h/mL to 1300 μg·h/mL, such as 1160 μg·h/mL to 1300 μg·h/mL, such as 1180 μg·h/mL to 1300 μg·h/mL, such as 1200 μg·h/mL to 1300 μg·h/mL, such as 1220 μg·h/mL to 1300 μg·h/mL, such as 1240 μg·h/mL to 1300 μg·h/mL, such as 1260 μg·h/mL to 1300 μg·h/mL, or such as 1280 μg·h/mL to 1300 μg·h/mL, in the subject.

Embodiment CLXXXVI. The trofinetide for use of Embodiment CLXXXV, wherein the administration of trofinetide results in an AUC_{0-12, ss} of 755 μg·h/mL to 1300 μg·h/mL in the subject.

Embodiment CLXXXVII. The trofinetide for use of Embodiment CLXXXVI, wherein the administration of trofinetide results in an AUC_{0-12, ss} of 780 μg·h/mL to 1300 μg·h/mL in the subject.

Embodiment CLXXXVIII. The trofinetide for use of Embodiment CLXXXVII, wherein the administration of trofinetide results in an AUC_{0-12, ss} of 790 μg·h/mL to 1300 μg·h/mL in the subject.

Embodiment CLXXXIX. The use of any one of Embodiments LXI-LXXI or LXXIV-XC, wherein the administration of trofinetide results in an AUC_{0-12, ss} of at least 755 μg·h/mL in the subject, such as 755 μg·h/mL to 1300 μg·h/mL, such as 780 μg·h/mL to 1300 μg·h/mL, such as 790 μg·h/mL to 1300 μg·h/mL, such as 800 μg·h/mL to 1300 μg·h/mL, such as 820 μg·h/mL to 1300 μg·h/mL, such as 840 μg·h/mL to 1300 μg·h/mL, such as 860 μg·h/mL to 1300 μg·h/mL, such as 880 μg·h/mL to 1300 μg·h/mL, such as 900 μg·h/mL to 1300 μg·h/mL, such as 920 μg·h/mL to 1300 μg·h/mL, such as 940 μg·h/mL to 1300 μg·h/mL, such as 960 μg·h/mL to 1300 μg·h/mL, such as 980 μg·h/mL to 1300 μg·h/mL, such as 1000 μg·h/mL to 1300 μg·h/mL, such as 1020 μg·h/mL to 1300 μg·h/mL, such as 1040 μg·h/mL to 1300 μg·h/mL, such as 1060 μg·h/mL to 1300 μg·h/mL, such as 1080 μg·h/mL to 1300 μg·h/mL, such as 1100 μg·h/mL to 1300 μg·h/mL, such as 1120 μg·h/mL to 1300 μg·h/mL, such as 1140 μg·h/mL to 1300 μg·h/mL, such as 1160 μg·h/mL to 1300 μg·h/mL, such as 1180 μg·h/mL to 1300 μg·h/mL, such as 1200 μg·h/mL to 1300 μg·h/mL, such as 1220 μg·h/mL to 1300 μg·h/mL, such as 1240 μg·h/mL to 1300 μg·h/mL, such as 1260 μg·h/mL to 1300 μg·h/mL, or such as 1280 μg·h/mL to 1300 μg·h/mL, in the subject.

Embodiment CXC. The use of Embodiment CLXXXIX, wherein the administration of trofinetide results in an AUC_{0-12, ss} of 755 μg·h/mL to 1300 μg·h/mL in the subject.

Embodiment CXCI. The use of Embodiment CXC, wherein the administration of trofinetide results in an AUC_{0-12, ss} of 780 μg·h/mL to 1300 μg·h/mL in the subject.

Embodiment CXCII. The use of Embodiment CXCI, wherein the administration of trofinetide results in an AUC_{0-12, ss} of 790 μg·h/mL to 1300 μg·h/mL in the subject.

Embodiment CXCIII. The use of any one of Embodiments XCI-XCIV or CII-CXX, wherein the administration of trofinetide results in an AUC_{0-12, ss} of at least 755 μg·h/mL in the subject, such as 755 μg·h/mL to 1300 μg·h/mL, such as 780 μg·h/mL to 1300 μg·h/mL, such as 790 μg·h/mL to 1300 μg·h/mL, such as 800 μg·h/mL to 1300 μg·h/mL, such as 820 μg·h/mL to 1300 μg·h/mL, such as 840 μg·h/mL to 1300 μg·h/mL, such as 860 μg·h/mL to 1300 μg·h/mL, such as 880 μg·h/mL to 1300 μg·h/mL, such as 900 μg·h/mL to 1300 μg·h/mL, such as 920 μg·h/mL to 1300 μg·h/mL, such as 940 μg·h/mL to 1300 μg·h/mL, such as 960 μg·h/mL to 1300 μg·h/mL, such as 980 μg·h/mL to 1300 μg·h/mL, such as 1000 μg·h/mL to 1300 μg·h/mL, such as 1020 μg·h/mL to 1300 μg·h/mL, such as 1040 μg·h/mL to 1300 μg·h/mL, such as 1060 μg·h/mL to 1300 μg·h/mL, such as 1080 μg·h/mL to 1300 μg·h/mL, such as 1100 μg·h/mL to 1300 μg·h/mL, such as 1120 μg·h/mL to 1300 μg·h/mL, such as 1140 μg·h/mL to 1300 μg·h/mL, such as 1160 μg·h/mL to 1300 μg·h/mL, such as 1180 μg·h/mL to 1300 μg·h/mL, such as 1200 μg·h/mL to 1300 μg·h/mL, such as 1220 μg·h/mL to 1300 μg·h/mL, such as 1240 μg·h/mL to 1300 μg·h/mL, such as 1260 μg·h/mL to 1300 μg·h/mL, or such as 1280 μg·h/mL to 1300 μg·h/mL, in the subject.

Embodiment CXCIV. The use of Embodiment CXCIII, wherein the administration of trofinetide results in an AUC_{0-12, ss} of 755 μg·h/mL to 1300 μg·h/mL in the subject.

Embodiment CXCV. The use of Embodiment CXCIV, wherein the administration of trofinetide results in an AUC_{0-12, ss} of 780 μg·h/mL to 1300 μg·h/mL in the subject.

Embodiment CXCVI. The use of Embodiment CXCV, wherein the administration of trofinetide results in an AUC_{0-12, ss} of 790 μg·h/mL to 1300 μg·h/mL in the subject.

Embodiment CXCVII. The trofinetide for use of any one of Embodiments CXXI-CXXIX or CXXXII-CL, wherein the administration of trofinetide results in an AUC_{0-12, ss} of at least 755 μg·h/mL in the subject, such as 755 μg·h/mL to 1300 μg·h/mL, such as 780 μg·h/mL to 1300 μg·h/mL, such as 790 μg·h/mL to 1300 μg·h/mL, such as 800 μg·h/mL to 1300 μg·h/mL, such as 820 μg·h/mL to 1300 μg·h/mL, such as 840 μg·h/mL to 1300 μg·h/mL, such as 860 μg·h/mL to 1300 μg·h/mL, such as 880 μg·h/mL to 1300 μg·h/mL, such as 900 μg·h/mL to 1300 μg·h/mL, such as 920 μg·h/mL to 1300 μg·h/mL, such as 940 μg·h/mL to 1300 μg·h/mL, such as 960 μg·h/mL to 1300 μg·h/mL, such as 980 μg·h/mL to 1300 μg·h/mL, such as 1000 μg·h/mL to 1300 μg·h/mL, such as 1020 μg·h/mL to 1300 μg·h/mL, such as 1040 μg·h/mL to 1300 μg·h/mL, such as 1060 μg·h/mL to 1300 μg·h/mL, such as 1080 μg·h/mL to 1300 μg·h/mL, such as 1100 μg·h/mL to 1300 μg·h/mL, such as 1120 μg·h/mL to 1300 μg·h/mL, such as 1140 μg·h/mL to 1300 μg·h/mL, such as 1160 μg·h/mL to 1300 μg·h/mL, such as 1180 μg·h/mL to 1300 μg·h/mL, such as 1200 μg·h/mL to 1300 μg·h/mL, such as 1220 μg·h/mL to 1300 μg·h/mL, such as 1240 μg·h/mL to 1300 μg·h/mL, such as 1260 μg·h/mL to 1300 μg·h/mL, or such as 1280 μg·h/mL to 1300 μg·h/mL, in the subject.

Embodiment CXCVIII. The trofinetide for use of Embodiment CXCVII, wherein the administration of trofinetide results in an AUC_{0-12, ss} of 755 μg·h/mL to 1300 μg·h/mL in the subject.

Embodiment CXCIX. The trofinetide for use of Embodiment CXCVIII, wherein the administration of trofinetide results in an AUC_{0-12, ss} of 780 μg·h/mL to 1300 μg·h/mL in the subject.

Embodiment CC. The trofinetide for use of Embodiment CXCIX, wherein the administration of trofinetide results in an AUC_{0-12, ss} of 790 μg·h/mL to 1300 μg·h/mL in the subject.

Embodiment CCI. The use of any one of Embodiments CLI-CLIX or CLXII-CLXXX, wherein the administration of trofinetide results in an AUC_{0-12, ss} of at least 755 μg·h/mL in the subject, such as 755 μg·h/mL to 1300 μg·h/mL, such as 780 μg·h/mL to 1300 μg·h/mL, such as 790 μg·h/mL to 1300 μg·h/mL, such as 800 μg·h/mL to 1300 μg·h/mL, such as 820 μg·h/mL to 1300 μg·h/mL, such as 840 μg·h/mL to 1300 μg·h/mL, such as 860 μg·h/mL to 1300 μg·h/mL, such as 880 μg·h/mL to 1300 μg·h/mL, such as 900 μg·h/mL to 1300 μg·h/mL, such as 920 μg·h/mL to 1300 μg·h/mL, such as 940 μg·h/mL to 1300 μg·h/mL, such as 960 μg·h/mL to 1300 μg·h/mL, such as 980 μg·h/mL to 1300 μg·h/mL, such as 1000 μg·h/mL to 1300 μg·h/mL, such as 1020 μg·h/mL to 1300 μg·h/mL, such as 1040 μg·h/mL to 1300 μg·h/mL, such as 1060 μg·h/mL to 1300 μg·h/mL, such as 1080 μg·h/mL to 1300 μg·h/mL, such as 1100 μg·h/mL to 1300 μg·h/mL, such as 1120 μg·h/mL to 1300 μg·h/mL, such as 1140 μg·h/mL to 1300 μg·h/mL, such as 1160 μg·h/mL to 1300 μg·h/mL, such as 1180 μg·h/mL to 1300 μg·h/mL, such as 1200 μg·h/mL to 1300 μg·h/mL, such as 1220 μg·h/mL to 1300 μg·h/mL, such as 1240 μg·h/mL to 1300 μg·h/mL, such as 1260 μg·h/mL to 1300 μg·h/mL, or such as 1280 μg·h/mL to 1300 μg·h/mL, in the subject.

Embodiment CCII. The use of Embodiment CCI, wherein the administration of trofinetide results in an AUC_{0-12, ss} of 755 μg·h/mL to 1300 μg·h/mL in the subject.

Embodiment CCIII. The use of Embodiment CCII, wherein the administration of trofinetide results in an AUC_{0-12, ss} of 780 μg·h/mL to 1300 μg·h/mL in the subject.

Embodiment CCIV. The use of Embodiment CCIII, wherein the administration of trofinetide results in an AUC_{0-12, ss} of 790 μg·h/mL to 1300 μg·h/mL in the subject.

Embodiment CCV. The method of any one of Embodiments I-XIII, XVI-XXX, or CLXXXI-CLXXXIV, wherein the administration of trofinetide to the subject results in a C_{max, ss} of at least 100 μg/mL in the subject, such as at 100 μg/mL to 300 μg/mL, such as 110 μg/mL to 300 μg/mL, such as 120 μg/mL to 300 μg/mL, such as 130 μg/mL to 300 μg/mL, such as 140 μg/mL to 300 μg/mL, such as 150 μg/mL to 300 μg/mL, such as 160 μg/mL to 300 μg/mL, such as 170 μg/mL to 300 g/mL, such as 180 g/mL to 300 g/mL, such as 190 g/mL to 300 μg/mL, such as 200 μg/mL to 300 μg/mL, such as 210 μg/mL to 300 μg/mL, such as 220 μg/mL to 300 μg/mL, such as 230 μg/mL to 300 μg/mL, such as 240 μg/mL to 300 μg/mL, such as 250 μg/mL to 300 μg/mL, such as 260 μg/mL to 300 μg/mL, such as 270 μg/mL to 300 μg/mL, such as 280 μg/mL to 300 μg/mL, or such as 290 μg/mL to 300 μg/mL, in the subject.

Embodiment CCVI. The trofinetide for use of any one of Embodiments XXXI-XLIII, XLVI-LX, or CLXXXV-CLXXXVIII, wherein the administration of trofinetide to the subject results in a C_{max, ss} of at least 100 μg/mL in the subject, such as at 100 μg/mL to 300 μg/mL, such as 110 μg/mL to 300 μg/mL, such as 120 μg/mL to 300 μg/mL, such as 130 μg/mL to 300 μg/mL, such as 140 μg/mL to 300 μg/mL, such as 150 μg/mL to 300 μg/mL, such as 160 μg/mL to 300 μg/mL, such as 170 μg/mL to 300 μg/mL, such as 180 μg/mL to 300 μg/mL, such as 190 μg/mL to 300 μg/mL, such as 200 μg/mL to 300 μg/mL, such as 210 μg/mL to 300 μg/mL, such as 220 μg/mL to 300 μg/mL, such as 230 μg/mL to 300 μg/mL, such as 240 μg/mL to 300 μg/mL, such as 250 μg/mL to 300 μg/mL, such as 260 μg/mL to 300 μg/mL, such as 270 μg/mL to 300 μg/mL, such as 280 μg/mL to 300 μg/mL, or such as 290 μg/mL to 300 μg/mL, in the subject

Embodiment CCVII. The use of any one of Embodiments LXI-LXXIII, LXXVI-XC, or CLXXXIX-CXCI, wherein the administration of trofinetide to the subject results in a C_{max, ss} of at least 100 μg/mL in the subject, such as at 100 μg/mL to 300 μg/mL, such as 110 μg/mL to 300 μg/mL, such as 120 μg/mL to 300 μg/mL, such as 130 μg/mL to 300 μg/mL, such as 140 μg/mL to 300 μg/mL, such as 150 μg/mL to 300 μg/mL, such as 160 μg/mL to 300 μg/mL, such as 170 μg/mL to 300 μg/mL, such as 180 μg/mL to 300 μg/mL, such as 190 μg/mL to 300 μg/mL, such as 200 μg/mL to 300 μg/mL, such as 210 μg/mL to 300 μg/mL, such as 220 μg/mL to 300 μg/mL, such as 230 μg/mL to 300 μg/mL, such as 240 μg/mL to 300 μg/mL, such as 250 μg/mL to 300 μg/mL, such as 260 μg/mL to 300 μg/mL, such as 270 μg/mL to 300 μg/mL, such as 280 μg/mL to 300 μg/mL, or such as 290 μg/mL to 300 μg/mL, in the subject.

Embodiment CCVIII. The method of any one of Embodiments XCI-CI, CIV-CXX, or CXCIII-CXCVI, wherein the administration of trofinetide to the subject results in a C_{max, ss} of at least 100 μg/mL in the subject, such as at 100 μg/mL to 300 μg/mL, such as 110 μg/mL to 300 μg/mL, such as 120 μg/mL to 300 μg/mL, such as 130 μg/mL to 300 μg/mL, such as 140 μg/mL to 300 μg/mL, such as 150 μg/mL to 300 μg/mL, such as 160 μg/mL to 300 μg/mL, such as 170 μg/mL to 300 μg/mL, such as 180 μg/mL to 300 μg/mL, such as 190 μg/mL to 300 μg/mL, such as 200 μg/mL to 300 μg/mL, such as 210 μg/mL to 300 μg/mL, such as 220 μg/mL to 300 μg/mL, such as 230 μg/mL to 300 μg/mL, such as 240 μg/mL to 300 μg/mL, such as 250 μg/mL to 300 μg/mL, such as 260 μg/mL to 300 μg/mL, such as 270 μg/mL to 300 μg/mL, such as 280 μg/mL to 300 μg/mL, or such as 290 μg/mL to 300 μg/mL, in the subject.

Embodiment CCIX. The trofinetide for use of any one of Embodiments CXXI-CXXXI, CXXXIV-CL, or CXCVII-CC, wherein the administration of trofinetide to the subject results in a C_{max, ss} of at least 100 μg/mL in the subject, such as at 100 μg/mL to 300 μg/mL, such as 110 μg/mL to 300 μg/mL, such as 120 μg/mL to 300 μg/mL, such as 130 μg/mL to 300 μg/mL, such as 140 μg/mL to 300 μg/mL, such as 150 μg/mL to 300 μg/mL, such as 160 μg/mL to 300 μg/mL, such as 170 μg/mL to 300 μg/mL, such as 180 μg/mL to 300 μg/mL, such as 190 μg/mL to 300 μg/mL, such as 200 μg/mL to 300 μg/mL, such as 210 μg/mL to 300 μg/mL, such as 220 μg/mL to 300 μg/mL, such as 230 μg/mL to 300 μg/mL, such as 240 μg/mL to 300 μg/mL, such as 250 μg/mL to 300 μg/mL, such as 260 μg/mL to 300 μg/mL, such as 270 μg/mL to 300 μg/mL, such as 280 μg/mL to 300 μg/mL, or such as 290 μg/mL to 300 μg/mL, in the subject

Embodiment CCX. The use of any one of Embodiments CLI-CLXI, CLXIV-CLXXX, or CCI-CCIV, wherein the administration of trofinetide to the subject results in a C_{max, ss} of at least 100 μg/mL in the subject, such as at 100 μg/mL to 300 μg/mL, such as 110 μg/mL to 300 μg/mL, such as 120 μg/mL to 300 μg/mL, such as 130 μg/mL to 300 μg/mL, such as 140 μg/mL to 300 μg/mL, such as 150 μg/mL to 300 μg/mL, such as 160 μg/mL to 300 μg/mL, such as 170 μg/mL to 300 μg/mL, such as 180 μg/mL to 300 μg/mL, such as 190 μg/mL to 300 μg/mL, such as 200 μg/mL to 300 μg/mL, such as 210 μg/mL to 300 μg/mL, such as 220 μg/mL to 300 μg/mL, such as 230 μg/mL to 300 μg/mL, such as 240 μg/mL to 300 μg/mL, such as 250 μg/mL to 300 μg/mL, such as 260 μg/mL to 300 μg/mL, such as 270 μg/mL to 300 μg/mL, such as 280 μg/mL to 300 μg/mL, or such as 290 μg/mL to 300 μg/mL, in the subject.

The following embodiments are also provided.

Embodiment 1. A method of treating Rett syndrome in a subject in need thereof, the method comprising administering to the subject a daily amount of trofinetide that provides an AUC_{0-12, ss} of about 755 μg·h/mL to about 1300 μg·h/mL, e.g., 755 μg·h/mL to 1300 μg·h/mL, in the subject.

Embodiment 2. The method of Embodiment 1, comprising administering to the subject a daily amount of trofinetide that provides an AUC_{0-12, ss} of about 800 μg·h/mL to about 1000 μg·h/mL, e.g., 800 μg·h/mL to 1000 μg·h/mL, in the subject.

Embodiment 3. A method of treating Rett syndrome in a subject in need thereof, the method comprising administering to the subject a daily amount of trofinetide that provides an AUC_{0-12, ss} of at least about 755 μg·h/mL, at least about 780 μg·h/mL, at least about 790 μg·h/mL, at least about 800 μg·h/mL, at least about 820 μg·h/mL, at least about 840 μg·h/mL, at least about 860 μg·h/mL, at least about 880 μg·h/mL, at least about 900 μg·h/mL, at least about 920 μg·h/mL, at least about 940 μg·h/mL, at least about 960 μg·h/mL, at least about 980 μg·h/mL, at least about 1000 μg·h/mL, at least about 1020 μg·h/mL, at least about 1040 μg·h/mL, at least about 1060 μg·h/mL, at least about 1080 μg·h/mL, at least about 1100 μg·h/mL, at least about 1120 μg·h/mL, at least about 1140 μg·h/mL, at least about 1160 μg·h/mL, at least about 1180 μg·h/mL, at least about 1200 μg·h/mL, at least about 1220 μg·h/mL, at least about 1240 μg·h/mL, at least about 1260 μg·h/mL, or at least about 1280 μg·h/mL in the subject.

Embodiment 4. The method of any one of Embodiments 1-3, wherein the subject weighs between about 12 kg to about 20 kg.

Embodiment 5. The method of any one of Embodiments 1-4, wherein the daily amount of trofinetide is about 12 g.

Embodiment 6. The method of any one of Embodiments 1-3, wherein the subject weighs between about 20.1 kg to about 35 kg.

Embodiment 7. The method of any one of Embodiments 1, 2, 3, or 6, wherein the daily amount of trofinetide is about 16 g.

Embodiment 8. The method of any one of Embodiments 1-3, wherein the subject weighs between about 35.1 kg to about 50 kg.

Embodiment 9. The method of any one of Embodiments 1, 2, 3, or 8 wherein the daily amount of trofinetide is about 20 g.

Embodiment 10. The method of any one of Embodiments 1-3, wherein the subject weighs between about 50.1 kg to about 100 kg.

Embodiment 11. The method of any one of Embodiments 1, 2, 3, or 10 wherein the daily amount of trofinetide is about 24 g.

Embodiment 12. The method of any one of Embodiments 1-11, wherein trofinetide is administered to the subject in a single dose per day.

Embodiment 13. The method of any one of Embodiments 1-11, wherein trofinetide is administered to the subject in multiple doses per day that sum to the daily amount.

Embodiment 14. The method of Embodiment 13, wherein trofinetide is administered to the subject in two doses per day that sum to the daily amount.

Embodiment 15. The method of any one of Embodiments 1-14, wherein trofinetide is administered as a pharmaceutical composition comprising trofinetide and a pharmaceutically acceptable carrier.

Embodiment 16. The method of Embodiment 15, wherein the pharmaceutically acceptable carrier comprises water.

Embodiment 17. The method of Embodiment 16, wherein the pharmaceutical composition is a solution.

Embodiment 18. The method of Embodiment 17, wherein the concentration of trofinetide is about 0.05 g/mL-0.7 g/mL in the solution.

Embodiment 19. The method of Embodiment 18, wherein the concentration of trofinetide is about 0.2 g/mL in the solution.

Embodiment 20. The method of any one of Embodiments 1-19, wherein trofinetide is orally administered to the subject.

Embodiment 21. The method of any one of Embodiments 1-20, wherein the subject is human.

Embodiment 22. The method of Embodiment 21, wherein the subject is female.

Embodiment 23. The method of any one of Embodiments 1-22, wherein the subject is about 18 months to about 20 years old.

Embodiment 24. The method of any one of Embodiments 1-23, wherein the subject has a MECP2 mutation.

Embodiment 25. The method of any one of Embodiments 1-22, wherein the Rett syndrome is atypical Rett syndrome.

Embodiment 26. The method of any one of Embodiments 1-24, wherein the Rett syndrome is classic/typical Rett syndrome.

Embodiment 27. Trofinetide for use in treating Rett syndrome in a subject, wherein trofinetide is to be administered to the subject in a daily amount that provides an AUC_{0-12, ss} of about 755 μg·h/mL to about 1300 μg·h/mL.

Embodiment 28. The trofinetide for use of Embodiment 27, wherein trofinetide is to be administered to the subject a daily amount of trofinetide that provides an AUC_{0-12, ss} of about 800 μg·h/mL to about 1000 μg·h/mL.

Embodiment 29. Trofinetide for use in treating Rett syndrome in a subject, wherein trofinetide is to be administered to the subject in a daily amount that provides an AUC_{0-12, ss} of at least about 755 μg·h/mL, at least about 780 μg·h/mL, at least about 790 μg·h/mL, at least about 800 μg·h/mL, at least about 820 μg·h/mL, at least about 840 μg·h/mL, at least about 860 μg·h/mL, at least about 880 μg·h/mL, at least about 900 μg·h/mL, at least about 920 μg·h/mL, at least about 940 μg·h/mL, at least about 960 μg·h/mL, at least about 980 μg·h/mL, at least about 1000 μg·h/mL, at least about 1020 μg·h/mL, at least about 1040 μg·h/mL, at least about 1060 μg·h/mL, at least about 1080 μg·h/mL, at least about 1100 μg·h/mL, at least about 1120 μg·h/mL, at least about 1140 μg·h/mL, at least about 1160 μg·h/mL, at least about 1180 μg·h/mL, at least about 1200 μg·h/mL, at least about 1220 μg·h/mL, at least about 1240 μg·h/mL, at least about 1260 μg·h/mL, or at least about 1280 μg·h/mL in the subject.

Embodiment 30. The trofinetide for use of any one of Embodiments 27-29, wherein the subject weighs between about 12 kg to about 20 kg.

Embodiment 31. The trofinetide for use of any one of Embodiments 27-30, wherein the daily amount of trofinetide is about 12 g.

Embodiment 32. The trofinetide for use of any one of Embodiments 27-29, wherein the subject weighs between about 20.1 kg to about 35 kg.

Embodiment 33. The trofinetide for use of any one of Embodiments 27-29 or 32, wherein the daily amount of trofinetide is about 16 g.

Embodiment 34. The trofinetide for use of any one of Embodiments 27-29, wherein the subject weighs between about 35.1 kg to about 50 kg.

Embodiment 35. The trofinetide for use of any one of Embodiments 27-29, or 34, wherein the daily amount of trofinetide is about 20 g.

Embodiment 36. The trofinetide for use of any one of Embodiments 27-29, wherein the subject weighs between about 50.1 kg to about 100 kg.

Embodiment 37. The trofinetide for use of any one of Embodiments 27-29, or 36 wherein the daily amount of trofinetide is about 24 g.

Embodiment 38. The trofinetide for use of any one of Embodiments 27-37, wherein trofinetide is to be administered to the subject in a single dose per day.

Embodiment 39. The trofinetide for use of any one of Embodiments 27-37, wherein trofinetide is to be administered to the subject in multiple doses per day that sum to the daily amount.

Embodiment 40. The trofinetide for use of Embodiment 39, wherein trofinetide is to be administered to the subject in two doses per day that sum to the daily amount.

Embodiment 41. The trofinetide for use of any one of Embodiments 27-40, wherein trofinetide is to be administered as a pharmaceutical composition comprising trofinetide and a pharmaceutically acceptable carrier.

Embodiment 42. The trofinetide for use of Embodiment 41, wherein the pharmaceutically acceptable carrier comprises water.

Embodiment 43. The trofinetide for use of Embodiment 42, wherein the pharmaceutical composition is a solution.

Embodiment 44. The trofinetide for use of Embodiment 43, wherein the concentration of trofinetide is about 0.05 g/mL-0.7 g/mL in the solution.

Embodiment 45. The trofinetide for use of Embodiment 44, wherein the concentration of trofinetide is about 0.2 g/mL in the solution.

Embodiment 46. The trofinetide for use of any one of Embodiments 27-45, wherein trofinetide is to be orally administered to the subject.

Embodiment 47. The trofinetide for use of any one of Embodiments 27-46, wherein the subject is human.

Embodiment 48. The trofinetide for use of Embodiment 47, wherein the subject is female.

Embodiment 49. The trofinetide for use of any one of Embodiments 27-48, wherein the subject is about 18 months to about 20 years old.

Embodiment 50. The trofinetide for use of any one of Embodiments 27-49, wherein the subject has a MECP2 mutation.

Embodiment 51. The trofinetide for use of any one of Embodiments 27-50, wherein the Rett syndrome is atypical Rett syndrome.

Embodiment 52. The trofinetide for use of any one of Embodiments 27-50, wherein the Rett syndrome is classic/typical Rett syndrome.

Embodiment 53. Use of trofinetide for the manufacture of a medicament for treating Rett syndrome in a subject, wherein trofinetide is to be administered to the subject in a daily amount that provides an AUC_{0-12, ss} of about 755 μg·h/mL to about 1300 μg·h/mL.

Embodiment 54. The use of Embodiment 53, comprising administering to the subject a daily amount of trofinetide that provides an AUC_{0-12, ss} of about 800 μg·h/mL to about 1000 μg·h/mL.

Embodiment 55. Use of trofinetide for the manufacture of a medicament for treating Rett syndrome in a subject, wherein trofinetide is to be administered to the subject in a daily amount that provides an AUC_{0-12, ss} of at least about 755 μg·h/mL, at least about 780 μg·h/mL, at least about 790 μg·h/mL, at least about 800 μg·h/mL, at least about 820 μg·h/mL, at least about 840 μg·h/mL, at least about 860 μg·h/mL, at least about 880 μg·h/mL, at least about 900 μg·h/mL, at least about 920 μg·h/mL, at least about 940 μg·h/mL, at least about 960 μg·h/mL, at least about 980 μg·h/mL, at least about 1000 μg·h/mL, at least about 1020 μg·h/mL, at least about 1040 μg·h/mL, at least about 1060 μg·h/mL, at least about 1080 μg·h/mL, at least about 1100 μg·h/mL, at least about 1120 μg·h/mL, at least about 1140 μg·h/mL, at least about 1160 μg·h/mL, at least about 1180 μg·h/mL, at least about 1200 μg·h/mL, at least about 1220 μg·h/mL, at least about 1240 μg·h/mL, at least about 1260 μg·h/mL, or at least about 1280 μg·h/mL in the subject.

Embodiment 56. The use of any one of Embodiments 53-55, wherein the subject weighs between about 12 kg to about 20 kg.

Embodiment 57. The use of any one of Embodiments 53-56, wherein the daily amount of trofinetide is about 12 g.

Embodiment 58. The use of any one of Embodiments 53-55, wherein the subject weighs between about 20.1 kg to about 35 kg.

Embodiment 59. The use of any one of Embodiments 53-55, or 58, wherein the daily amount of trofinetide is about 16 g.

Embodiment 60. The use of any one of Embodiments 53-55, wherein the subject weighs between about 35.1 kg to about 50 kg.

Embodiment 61. The use of any one of Embodiments 53-55, or 60, wherein the daily amount of trofinetide is about 20 g.

Embodiment 62. The use of any one of Embodiments 53-55, wherein the subject weighs between about 50.1 kg to about 100 kg.

Embodiment 63. The use of any one of Embodiments 53-55, or 62 wherein the daily amount of trofinetide is about 24 g.

Embodiment 64. The use of any one of Embodiments 53-63, wherein trofinetide is to be administered to the subject in a single dose per day.

Embodiment 65. The use any one of Embodiments 53-63, wherein trofinetide is to be administered to the subject in multiple doses per day that sum to the daily amount.

Embodiment 66. The use of Embodiment 65, wherein trofinetide is to be administered to the subject in two doses per day that sum to the daily amount.

Embodiment 67. The use of any one of Embodiments 53-66, wherein trofinetide is to be administered as a pharmaceutical composition comprising trofinetide and a pharmaceutically acceptable carrier.

Embodiment 68. The use of Embodiment 67, wherein the pharmaceutically acceptable carrier comprises water.

Embodiment 69. The use of Embodiment 68, wherein the pharmaceutical composition is a solution.

Embodiment 70. The use of Embodiment 69, wherein the concentration of trofinetide is about 0.05 g/mL-0.7 g/mL in the solution.

Embodiment 71. The use of Embodiment 70, wherein the concentration of trofinetide is about 0.2 g/mL in the solution.

Embodiment 72. The use of any one of Embodiments 53-71, wherein trofinetide is to be orally administered to the subject.

Embodiment 73. The use of any one of Embodiments 53-72, wherein the subject is human.

Embodiment 74. The use of Embodiment 73, wherein the subject is female.

Embodiment 75. The use of any one of Embodiments 53-74, wherein the subject is about 18 months to about 20 years old.

Embodiment 76. The use of any one of Embodiments 53-75, wherein the subject has a MECP2 mutation.

Embodiment 77. The use of any one of Embodiments 53-76, wherein the Rett syndrome is atypical Rett syndrome.

Embodiment 78. The use of any one of Embodiments 53-76, wherein the Rett syndrome is classic/typical Rett syndrome.

Embodiment 79. The method of any one of Embodiments 3-26, wherein the administration of trofinetide results in an AUC_{0-12, ss} of at least 755 μg·h/mL, such as 755 μg·h/mL to 1300 μg·h/mL, such as 780 μg·h/mL to 1300 μg·h/mL, such as 790 μg·h/mL to 1300 μg·h/mL, such as 800 μg·h/mL to 1300 μg·h/mL, such as 820 μg·h/mL to 1300 μg·h/mL, such as 840 μg·h/mL to 1300 μg·h/mL, such as 860 μg·h/mL to 1300 μg·h/mL, such as 880 μg·h/mL to 1300 μg·h/mL, such as 900 μg·h/mL to 1300 μg·h/mL, such as 920 μg·h/mL to 1300 μg·h/mL, such as 940 μg·h/mL to 1300 μg·h/mL, such as 960 μg·h/mL to 1300 μg·h/mL, such as 980 μg·h/mL to 1300 μg·h/mL, such as 1000 μg·h/mL to 1300 μg·h/mL, such as 1020 μg·h/mL to 1300 μg·h/mL, such as 1040 μg·h/mL to 1300 μg·h/mL, such as 1060 μg·h/mL to 1300 μg·h/mL, such as 1080 μg·h/mL to 1300 μg·h/mL, such as 1100 μg·h/mL to 1300 μg·h/mL, such as 1120 μg·h/mL to 1300 μg·h/mL, such as 1140 μg·h/mL to 1300 μg·h/mL, such as 1160 μg·h/mL to 1300 μg·h/mL, such as 1180 μg·h/mL to 1300 μg·h/mL, such as 1200 μg·h/mL to 1300 μg·h/mL, such as 1220 μg·h/mL to 1300 μg·h/mL, such as 1240 μg·h/mL to 1300 μg·h/mL, such as 1260 μg·h/mL to 1300 μg·h/mL, or such as 1280 μg·h/mL to 1300 μg·h/mL, in the subject.

Embodiment 80. The method of Embodiment 79, wherein the administration of trofinetide results in an AUC_{0-12, ss} of 755 μg·h/mL to 1300 μg·h/mL in the subject.

Embodiment 81. The method of Embodiment 80, wherein the administration of trofinetide results in an AUC_{0-12, ss} of 780 μg·h/mL to 1300 μg·h/mL in the subject.

Embodiment 82. The method of Embodiment 81, wherein the administration of trofinetide results in an AUC_{0-12, ss} of 790 μg·h/mL to 1300 μg·h/mL in the subject.

Embodiment 83. The trofinetide for use of any one of Embodiments 29-52, wherein the administration of trofinetide results in an AUC_{0-12, ss} of at least 755 μg·h/mL, such as 755 μg·h/mL to 1300 μg·h/mL, such as 780 μg·h/mL to 1300 μg·h/mL, such as 790 μg·h/mL to 1300 μg·h/mL, such as 800 μg·h/mL to 1300 μg·h/mL, such as 820 μg·h/mL to 1300 μg·h/mL, such as 840 μg·h/mL to 1300 μg·h/mL, such as 860 μg·h/mL to 1300 μg·h/mL, such as 880 μg·h/mL to 1300 μg·h/mL, such as 900 μg·h/mL to 1300 μg·h/mL, such as 920 μg·h/mL to 1300 μg·h/mL, such as 940 μg·h/mL to 1300 μg·h/mL, such as 960 μg·h/mL to 1300 μg·h/mL, such as 980 μg·h/mL to 1300 μg·h/mL, such as 1000 μg·h/mL to 1300 μg·h/mL, such as 1020 μg·h/mL to 1300 μg·h/mL, such as 1040 μg·h/mL to 1300 μg·h/mL, such as 1060 μg·h/mL to 1300 μg·h/mL, such as 1080 μg·h/mL to 1300 μg·h/mL, such as 1100 μg·h/mL to 1300 μg·h/mL, such as 1120 μg·h/mL to 1300 μg·h/mL, such as 1140 μg·h/mL to 1300 μg·h/mL, such as 1160 μg·h/mL to 1300 μg·h/mL, such as 1180 μg·h/mL to 1300 μg·h/mL, such as 1200 μg·h/mL to 1300 μg·h/mL, such as 1220 μg·h/mL to 1300 μg·h/mL, such as 1240 μg·h/mL to 1300 μg·h/mL, such as 1260 μg·h/mL to 1300 μg·h/mL, or such as 1280 μg·h/mL to 1300 μg·h/mL, in the subject.

Embodiment 84. The trofinetide for use of Embodiment 83, wherein the administration of trofinetide results in an AUC_{0-12, ss} of 755 μg·h/mL to 1300 μg·h/mL in the subject.

Embodiment 85. The trofinetide for use of Embodiment 84, wherein the administration of trofinetide results in an AUC_{0-12, ss} of 780 μg·h/mL to 1300 μg·h/mL in the subject.

Embodiment 86. The trofinetide for use of Embodiment 85, wherein the administration of trofinetide results in an AUC_{0-12, ss} of 790 μg·h/mL to 1300 μg·h/mL in the subject.

Embodiment 87. The use of any one of Embodiments 55-78, wherein the administration of trofinetide results in an AUC_{0-12, ss} of at least 755 μg·h/mL, such as 755 μg·h/mL to 1300 μg·h/mL, such as 780 μg·h/mL to 1300 μg·h/mL, such as 790 μg·h/mL to 1300 μg·h/mL, such as 800 μg·h/mL to 1300 μg·h/mL, such as 820 μg·h/mL to 1300 μg·h/mL, such as 840 μg·h/mL to 1300 μg·h/mL, such as 860 μg·h/mL to 1300 μg·h/mL, such as 880 μg·h/mL to 1300 μg·h/mL, such as 900 μg·h/mL to 1300 μg·h/mL, such as 920 μg·h/mL to 1300 μg·h/mL, such as 940 μg·h/mL to 1300 μg·h/mL, such as 960 μg·h/mL to 1300 μg·h/mL, such as 980 μg·h/mL to 1300 μg·h/mL, such as 1000 μg·h/mL to 1300 μg·h/mL, such as 1020 μg·h/mL to 1300 μg·h/mL, such as 1040 μg·h/mL to 1300 μg·h/mL, such as 1060 μg·h/mL to 1300 μg·h/mL, such as 1080 μg·h/mL to 1300 μg·h/mL, such as 1100 μg·h/mL to 1300 μg·h/mL, such as 1120 μg·h/mL to 1300 μg·h/mL, such as 1140 μg·h/mL to 1300 μg·h/mL, such as 1160 μg·h/mL to 1300 μg·h/mL, such as 1180 μg·h/mL to 1300 μg·h/mL, such as 1200 μg·h/mL to 1300 μg·h/mL, such as 1220 μg·h/mL to 1300 μg·h/mL, such as 1240 μg·h/mL to 1300 μg·h/mL, such as 1260 μg·h/mL to 1300 μg·h/mL, or such as 1280 μg·h/mL to 1300 μg·h/mL, in the subject.

Embodiment 88. The use of Embodiment 87, wherein the administration of trofinetide results in an AUC_{0-12, ss} of 755 μg·h/mL to 1300 μg·h/mL in the subject.

Embodiment 89. The use of Embodiment 88, wherein the administration of trofinetide results in an AUC_{0-12, ss} of 780 μg·h/mL to 1300 μg·h/mL in the subject.

Embodiment 90. The use of Embodiment 89, wherein the administration of trofinetide results in an AUC_{0-12, ss} of 790 μg·h/mL to 1300 μg·h/mL in the subject.

The following embodiments are also provided.

Embodiment 1. A method of treating Rett syndrome in a subject in need thereof, the method comprising administering to the subject a daily amount of trofinetide that provides a C_{max, ss} of about 100 μg/mL to about 300 μg/mL in the subject.

Embodiment 2. The method of Embodiment 1, comprising administering to the subject a daily amount of trofinetide that provides a C_{max, ss} of about 100 μg/mL to about 200 μg/mL in the subject.

Embodiment 3. A method of treating Rett syndrome in a subject in need thereof, the method comprising administering to the subject a daily amount of trofinetide that provides a C_{max, ss} of at least about 100 μg/mL, at least about 110 μg/mL, at least about 120 μg/mL, at least about 130 μg/mL, at least about 140 μg/mL, at least about 150 μg/mL, at least about 160 μg/mL, at least about 170 μg/mL, at least about 180 μg/mL, at least about 190 μg/mL, or at least about 200 μg/mL in the subject.

Embodiment 4. The method of any one of Embodiments 1-3, wherein the subject weighs between about 12 kg to about 20 kg.

Embodiment 5. The method of any one of Embodiments 1-4, wherein the daily amount of trofinetide is about 12 g.

Embodiment 6. The method of any one of Embodiments 1-3, wherein the subject weighs between about 20.1 kg to about 35 kg.

Embodiment 7. The method of Embodiments 1, 2, 3, or 6, wherein the daily amount of trofinetide is about 16 g.

Embodiment 8. The method of any one of Embodiments 1-3, wherein the subject weighs between about 35.1 kg to about 50 kg.

Embodiment 9. The method of Embodiments 1, 2, 3, or 8 wherein the daily amount of trofinetide is about 20 g.

Embodiment 10. The method of any one of Embodiments 1-32, wherein the subject weighs between about 50.1 kg to about 100 kg.

Embodiment 11. The method of Embodiments 1, 2, 3, or 10 wherein the daily amount of trofinetide is about 24 g.

Embodiment 12. The method of any one of Embodiments 1-11, wherein trofinetide is administered to the subject in a single dose per day.

Embodiment 13. The method of any one of Embodiments 1-11, wherein trofinetide is administered to the subject in multiple doses per day that sum to the daily amount.

Embodiment 14. The method of Embodiment 13, wherein trofinetide is administered to the subject in two doses per day that sum to the daily amount.

Embodiment 15. The method of any one of Embodiments 1-14, wherein trofinetide is administered as a pharmaceutical composition comprising trofinetide and a pharmaceutically acceptable carrier.

Embodiment 16. The method of Embodiment 15, wherein the pharmaceutically acceptable carrier comprises water.

Embodiment 17. The method of Embodiment 16, wherein the pharmaceutical composition is a solution.

Embodiment 18. The method of Embodiment 17, wherein the concentration of trofinetide is about 0.05 g/mL-0.7 g/mL in the solution.

Embodiment 19. The method of Embodiment 18, wherein the concentration of trofinetide is about 0.2 g/mL in the solution.

Embodiment 20. The method of any one of Embodiments 1-19, wherein trofinetide is orally administered to the subject.

Embodiment 21. The method of any one of Embodiments 1-20, wherein the subject is human.

Embodiment 22. The method of Embodiment 21, wherein the subject is female.

Embodiment 23. The method of any one of Embodiments 1-22, wherein the subject is about 18 months to about 20 years old.

Embodiment 24. The method of any one of Embodiments 1-23, wherein the subject has a MECP2 mutation.

Embodiment 25. The method of any one of Embodiments 1-22, wherein the Rett syndrome is atypical Rett syndrome.

Embodiment 26. The method of any one of Embodiments 1-24, wherein the Rett syndrome is classic/typical Rett syndrome.

Embodiment 27. Trofinetide for use in treating Rett syndrome in a subject, wherein trofinetide is to be administered to the subject in a daily amount that provides a C_{max, ss} of about 100 μg/mL to about 300 μg/mL in the subject.

Embodiment 28. The trofinetide for use of Embodiment 27, wherein trofinetide is to be administered to the subject a daily amount of trofinetide that provides a C_{max, ss} of about 100 μg/mL to about 200 μg/mL in the subject.

Embodiment 29. Trofinetide for use in treating Rett syndrome in a subject, wherein trofinetide is to be administered to the subject in a daily amount that provides a C_{max, ss} of at least about 100 μg/mL, at least about 110 μg/mL, at least about 120 μg/mL, at least about 130 μg/mL, at least about 140 μg/mL, at least about 150 μg/mL, at least about 160 μg/mL, at least about 170 μg/mL, at least about 180 μg/mL, at least about 190 μg/mL, or at least about 200 μg/mL in the subject.

Embodiment 30. The trofinetide for use of any one of Embodiments 27-29, wherein the subject weighs between about 12 kg to about 20 kg.

Embodiment 31. The trofinetide for use of any one of Embodiments 27-30, wherein the daily amount of trofinetide is about 12 g.

Embodiment 32. The trofinetide for use of any one of Embodiments 27-29, wherein the subject weighs between about 20.1 kg to about 35 kg.

Embodiment 33. The trofinetide for use of any one of Embodiments 27-29 or 32, wherein the daily amount of trofinetide is about 16 g.

Embodiment 34. The trofinetide for use of any one of Embodiments 27-29, wherein the subject weighs between about 35.1 kg to about 50 kg.

Embodiment 35. The trofinetide for use of any one of Embodiments 27-29, or 34, wherein the daily amount of trofinetide is about 20 g.

Embodiment 36. The trofinetide for use of any one of Embodiments 27-29, wherein the subject weighs between about 50.1 kg to about 100 kg.

Embodiment 37. The trofinetide for use of any one of Embodiments 27-29, or 36 wherein the daily amount of trofinetide is about 24 g.

Embodiment 38. The trofinetide for use of any one of Embodiments 27-37, wherein trofinetide is to be administered to the subject in a single dose per day.

Embodiment 39. The trofinetide for use of any one of Embodiments 27-37, wherein trofinetide is to be administered to the subject in multiple doses per day that sum to the daily amount.

Embodiment 40. The trofinetide for use of Embodiment 39, wherein trofinetide is to be administered to the subject in two doses per day that sum to the daily amount.

Embodiment 41. The trofinetide for use of any one of Embodiments 27-40, wherein trofinetide is to be administered as a pharmaceutical composition comprising trofinetide and a pharmaceutically acceptable carrier.

Embodiment 42. The trofinetide for use of Embodiment 41, wherein the pharmaceutically acceptable carrier comprises water.

Embodiment 43. The trofinetide for use of Embodiment 42, wherein the pharmaceutical composition is a solution.

Embodiment 44. The trofinetide for use of Embodiment 43, wherein the concentration of trofinetide is about 0.05 g/mL-0.7 g/mL in the solution.

Embodiment 45. The trofinetide for use of Embodiment 44, wherein the concentration of trofinetide is about 0.2 g/mL in the solution.

Embodiment 46. The trofinetide for use of any one of Embodiments 27-45, wherein trofinetide is to be orally administered to the subject.

Embodiment 47. The trofinetide for use of any one of Embodiments 27-46, wherein the subject is human.

Embodiment 48. The trofinetide for use of Embodiment 47, wherein the subject is female.

Embodiment 49. The trofinetide for use of any one of Embodiments 27-48, wherein the subject is about 18 months to about 20 years old.

Embodiment 50. The trofinetide for use of any one of Embodiments 27-49, wherein the subject has a MECP2 mutation.

Embodiment 51. The trofinetide for use of any one of Embodiments 27-50, wherein the Rett syndrome is atypical Rett syndrome.

Embodiment 52. The trofinetide for use of any one of Embodiments 27-50, wherein the Rett syndrome is classic/typical Rett syndrome.

Embodiment 53. Use of trofinetide for the manufacture of a medicament for treating Rett syndrome in a subject, wherein trofinetide is to be administered to the subject in a daily amount that provides a C_{max, ss} of about 100 μg/mL to about 300 μg/mL in the subject.

Embodiment 54. The use of Embodiment 53, comprising administering to the subject a daily amount of trofinetide that provides a C_{max, ss} of about 100 μg/mL to about 200 μg/mL in the subject.

Embodiment 55. Use of trofinetide for the manufacture of a medicament for treating Rett syndrome in a subject, wherein trofinetide is to be administered to the subject in a daily amount that provides a C_{max, ss} of at least about 100 μg/mL, at least about 110 μg/mL, at least about 120 μg/mL, at least about 130 μg/mL, at least about 140 μg/mL, at least about 150 μg/mL, at least about 160 μg/mL, at least about 170 μg/mL, at least about 180 μg/mL, at least about 190 μg/mL, or at least about 200 μg/mL in the subject.

Embodiment 56. The use of any one of Embodiments 53-55, wherein the subject weighs between about 12 kg to about 20 kg.

Embodiment 57. The use of any one of Embodiments 53-56, wherein the daily amount of trofinetide is about 12 g.

Embodiment 58. The use of any one of Embodiments 53-55, wherein the subject weighs between about 20.1 kg to about 35 kg.

Embodiment 59. The use of any one of Embodiments 53-55, or 58, wherein the daily amount of trofinetide is about 16 g.

Embodiment 60. The use of any one of Embodiments 53-55, wherein the subject weighs between about 35.1 kg to about 50 kg.

Embodiment 61. The use of any one of Embodiments 53-55, or 60, wherein the daily amount of trofinetide is about 20 g.

Embodiment 62. The use of any one of Embodiments 53-55, wherein the subject weighs between about 50.1 kg to about 100 kg.

Embodiment 63. The use of any one of Embodiments 53-55, or 62 wherein the daily amount of trofinetide is about 24 g.

Embodiment 64. The use of any one of Embodiments 53-63, wherein trofinetide is to be administered to the subject in a single dose per day.

Embodiment 65. The use any one of Embodiments 53-63, wherein trofinetide is to be administered to the subject in multiple doses per day that sum to the daily amount.

Embodiment 66. The use of Embodiment 65, wherein trofinetide is to be administered to the subject in two doses per day that sum to the daily amount.

Embodiment 67. The use of any one of Embodiments 53-66, wherein trofinetide is to be administered as a pharmaceutical composition comprising trofinetide and a pharmaceutically acceptable carrier.

Embodiment 68. The use of Embodiment 67, wherein the pharmaceutically acceptable carrier comprises water.

Embodiment 69. The use of Embodiment 68, wherein the pharmaceutical composition is a solution.

Embodiment 70. The use of Embodiment 69, wherein the concentration of trofinetide is about 0.05 g/mL-0.7 g/mL in the solution.

Embodiment 71. The use of Embodiment 70, wherein the concentration of trofinetide is about 0.2 g/mL in the solution.

Embodiment 72. The use of any one of Embodiments 53-71, wherein trofinetide is to be orally administered to the subject.

Embodiment 73. The use of any one of Embodiments 53-72, wherein the subject is human.

Embodiment 74. The use of Embodiment 73, wherein the subject is female.

Embodiment 75. The use of any one of Embodiments 53-74, wherein the subject is about 18 months to about 20 years old.

Embodiment 76. The use of any one of Embodiments 53-75, wherein the subject has a MECP2 mutation.

Embodiment 77. The use of any one of Embodiments 53-76, wherein the Rett syndrome is atypical Rett syndrome.

Embodiment 78. The use of any one of Embodiments 53-76, wherein the Rett syndrome is classic/typical Rett syndrome.

Embodiment 79. The method of any one of Embodiments 3-26, wherein the administration of trofinetide to the subject results in a C_{max, ss} of at least 100 μg/mL in the subject, such as at 100 μg/mL to 300 μg/mL, such as 110 μg/mL to 300 μg/mL, such as 120 μg/mL to 300 μg/mL, such as 130 μg/mL to 300 μg/mL, such as 140 μg/mL to 300 μg/mL, such as 150 μg/mL to 300 μg/mL, such as 160 μg/mL to 300 μg/mL, such as 170 μg/mL to 300 μg/mL, such as 180 μg/mL to 300 μg/mL, such as 190 μg/mL to 300 μg/mL, such as 200 μg/mL to 300 μg/mL, such as 210 μg/mL to 300 μg/mL, such as 220 μg/mL to 300 μg/mL, such as 230 μg/mL to 300 μg/mL, such as 240 μg/mL to 300 μg/mL, such as 250 μg/mL to 300 μg/mL, such as 260 μg/mL to 300 μg/mL, such as 270 μg/mL to 300 μg/mL, such as 280 μg/mL to 300 μg/mL, or such as 290 μg/mL to 300 μg/mL, in the subject.

Embodiment 80. The trofinetide for use of any one of Embodiments 29-52, wherein the administration of trofinetide to the subject results in a C_{max, ss} of at least 100 μg/mL in the subject, such as at 100 μg/mL to 300 μg/mL, such as 110 μg/mL to 300 μg/mL, such as 120 μg/mL to 300 μg/mL, such as 130 μg/mL to 300 μg/mL, such as 140 μg/mL to 300 μg/mL, such as 150 μg/mL to 300 μg/mL, such as 160 μg/mL to 300 μg/mL, such as 170 μg/mL to 300 μg/mL, such as 180 μg/mL to 300 μg/mL, such as 190 μg/mL to 300 μg/mL, such as 200 μg/mL to 300 μg/mL, such as 210 μg/mL to 300 μg/mL, such as 220 μg/mL to 300 μg/mL, such as 230 μg/mL to 300 μg/mL, such as 240 μg/mL to 300 μg/mL, such as 250 μg/mL to 300 μg/mL, such as 260 μg/mL to 300 μg/mL, such as 270 μg/mL to 300 μg/mL, such as 280 μg/mL to 300 μg/mL, or such as 290 μg/mL to 300 μg/mL, in the subject

Embodiment 81. The use of any one of Embodiments 55-78, wherein the administration of trofinetide to the subject results in a C_{max, ss} of at least 100 μg/mL in the subject, such as at 100 μg/mL to 300 μg/mL, such as 110 μg/mL to 300 μg/mL, such as 120 μg/mL to 300 μg/mL, such as 130 μg/mL to 300 μg/mL, such as 140 μg/mL to 300 μg/mL, such as 150 μg/mL to 300 μg/mL, such as 160 μg/mL to 300 μg/mL, such as 170 μg/mL to 300 μg/mL, such as 180 μg/mL to 300 μg/mL, such as 190 μg/mL to 300 μg/mL, such as 200 μg/mL to 300 μg/mL, such as 210 μg/mL to 300 μg/mL, such as 220 μg/mL to 300 μg/mL, such as 230 μg/mL to 300 μg/mL, such as 240 μg/mL to 300 μg/mL, such as 250 μg/mL to 300 μg/mL, such as 260 μg/mL to 300 μg/mL, such as 270 μg/mL to 300 μg/mL, such as 280 μg/mL to 300 μg/mL, or such as 290 μg/mL to 300 μg/mL, in the subject.

The following embodiments are also provided.

Embodiment 1. A method of treating Rett syndrome in a subject in need thereof, the method comprising administering a therapeutically effective amount of trofinetide to the subject, wherein:

(i) a total daily dose of about 1 g to about 4 g of trofinetide is administered to the subject for the first 7±3 days of treatment Period A;

(ii) a total daily dose of about 4 g to about 6 g of trofinetide is administered to the subject for the next 14±3 days of treatment Period B;

(iii) a total daily dose of about 6 g to about 8 g of trofinetide is administered to the subject for the next 28±3 days of treatment Period C; and

(iv) a total daily dose of about 8 g to about 10 g of trofinetide is administered to the subject after completion of treatment Period C,

wherein the subject:

(a) is less than 4 years old at the time treatment Period A begins; and/or

(b) weighs between about 9 kg to about 12 kg at the time treatment Period A begins.

Embodiment 2. The method of Embodiment 1, wherein a total daily dose of about 1 g of trofinetide is administered to the subject for the 7±3 days of treatment Period A.

Embodiment 3. The method of Embodiment 1, wherein a total daily dose of about 2 g of trofinetide is administered to the subject for the 7±3 days of treatment Period A.

Embodiment 4. The method of Embodiment 1, wherein a total daily dose of about 3 g of trofinetide is administered to the subject for the 7±3 days of treatment Period A.

Embodiment 5. The method of Embodiment 1, wherein a total daily dose of about 4 g of trofinetide is administered to the subject for the 7±3 days of treatment Period A.

Embodiment 6. The method of any one of Embodiments 1-5, wherein a total daily dose of about 4 g of trofinetide is administered to the subject for the 14±3 days of treatment Period B.

Embodiment 7. The method of any one of Embodiments 1-5, wherein a total daily dose of about 5 g of trofinetide is administered to the subject for the 14 t 3 days of treatment Period B.

Embodiment 8. The method of any one of Embodiments 1-5, wherein a total daily dose of about 6 g of trofinetide is administered to the subject for the 14±3 days of treatment Period B.

Embodiment 9. The method of any one of Embodiments 1-8, wherein a total daily dose of about 6 g of trofinetide is administered to the subject for the 28±3 days of treatment Period C.

Embodiment 10. The method of any one of Embodiments 1-8, wherein about 7 g of trofinetide is administered to the subject for the 28±3 days of treatment Period C.

Embodiment 11. The method of any one of Embodiments 1-8, wherein a total daily dose of about 8 g of trofinetide is administered to the subject for the 28 t 3 days of treatment Period C.

Embodiment 12. The method of any one of Embodiments 1-11, wherein a total daily dose of about 8 g of trofinetide is administered to the subject after completion of treatment Period C.

Embodiment 13. The method of any one of Embodiments 1-11, wherein a total daily dose of about 9 g of trofinetide is administered to the subject after completion of treatment Period C.

Embodiment 14. The method of any one of Embodiments 1-11, wherein a total daily dose of about 10 g of trofinetide is administered to the subject after completion of treatment Period C.

Embodiment 15. The method of any one of Embodiments 1-14, wherein the subject experiences no treatment-emergent adverse events.

Embodiment 16. The method of any one of Embodiments 1-15, wherein trofinetide is administered to the subject in a single dose per day.

Embodiment 17. The method of any one of Embodiments 1-15, wherein trofinetide is administered to the subject in multiple doses per day that sum to the daily amount.

Embodiment 18. The method of Embodiment 17, wherein trofinetide is administered to the subject in two doses per day that sum to the daily amount.

Embodiment 19. The method of any one of Embodiments 1-18, wherein trofinetide is administered as a pharmaceutical composition comprising trofinetide and a pharmaceutically acceptable carrier.

Embodiment 20. The method of Embodiment 19, wherein the pharmaceutically acceptable carrier comprises water.

Embodiment 21. The method of Embodiment 20, wherein the pharmaceutical composition is a solution.

Embodiment 22. The method of Embodiment 21, wherein the concentration of trofinetide is about 0.05 g/mL-0.7 g/mL in the solution.

Embodiment 23. The method of Embodiment 22, wherein the concentration of trofinetide is about 0.2 g/mL in the solution.

Embodiment 24. The method of any one of Embodiments 1-23, wherein trofinetide is orally administered to the subject.

Embodiment 25. The method of any one of Embodiments 1-23, wherein trofinetide is administered by gastrostomy (G) tube to the subject.

Embodiment 26. The method of any one of Embodiments 1-25, wherein the subject is human.

Embodiment 27. The method of Embodiment 26, wherein the subject is female.

Embodiment 28. The method of any one of Embodiments 1-27, wherein the subject has a MECP2 mutation.

Embodiment 29. The method of any one of Embodiments 1-28, wherein the Rett syndrome is atypical Rett syndrome.

Embodiment 30. The method of any one of Embodiments 1-28, wherein the Rett syndrome is classic/typical Rett syndrome.

Embodiment 31. Trofinetide for use in treating Rett syndrome in a subject, wherein:

(i) a total daily dose of about 1 g to about 4 g of trofinetide is to be administered to the subject for the first 7±3 days of treatment Period A;

(ii) a total daily dose of about 4 g to about 6 g of trofinetide is to be administered to the subject for the next 14±3 days of treatment Period B;

(iii) a total daily dose of about 6 g to about 8 g of trofinetide is to be administered to the subject for the next 28±3 days of treatment Period C; and

(iv) a total daily dose of about 8 g to about 10 g of trofinetide is to be administered to the subject after completion of treatment Period C,

wherein the subject:

(a) is less than 4 years old at the time treatment Period A begins; and/or

(b) weighs between about 9 kg to about 12 kg at the time treatment Period A begins.

Embodiment 32. The trofinetide for use of Embodiment 31, wherein a total daily dose of about 1 g of trofinetide is to be administered to the subject for the 7±3 days of treatment Period A.

Embodiment 33. The trofinetide for use of Embodiment 31, wherein a total daily dose of about 2 g of trofinetide is to be administered to the subject for the 7±3 days of treatment Period A.

Embodiment 34. The trofinetide for use of Embodiment 31, wherein a total daily dose of about 3 g of trofinetide is to be administered to the subject for the 7±3 days of treatment Period A.

Embodiment 35. The trofinetide for use of Embodiment 31, wherein a total daily dose of about 4 g of trofinetide is to be administered to the subject for the 7±3 days of treatment Period A.

Embodiment 36. The trofinetide for use of any one of Embodiments 31-35, wherein a total daily dose of about 4 g of trofinetide is to be administered to the subject for the 14±3 days of treatment Period B.

Embodiment 37. The trofinetide for use of any one of Embodiments 31-35, wherein a total daily dose of about 5 g of trofinetide is to be administered to the subject for the 14±3 days of treatment Period B.

Embodiment 38. The trofinetide for use of any one of Embodiments 31-35, wherein a total daily dose of about 6 g of trofinetide is to be administered to the subject for the 14±3 days of treatment Period B.

Embodiment 39. The trofinetide for use of any one of Embodiments 31-38, wherein a total daily dose of about 6 g of trofinetide is to be administered to the subject for the 28±3 days of treatment Period C.

Embodiment 40. The trofinetide for use of any one of Embodiments 31-38, wherein about 7 g of trofinetide is to be administered to the subject for the 28±3 days of treatment Period C.

Embodiment 41. The trofinetide for use of any one of Embodiments 31-38, wherein a total daily dose of about 8 g of trofinetide is to be administered to the subject for the 28±3 days of treatment Period C.

Embodiment 42. The trofinetide for use of any one of Embodiments 31-41, wherein a total daily dose of about 8 g of trofinetide is to be administered to the subject after completion of treatment Period C.

Embodiment 43. The trofinetide for use of any one of Embodiments 31-41, wherein a total daily dose of about 9 g of trofinetide is to be administered to the subject after completion of treatment Period C.

Embodiment 44. The trofinetide for use of any one of Embodiments 31-41, wherein a total daily dose of about 10 g of trofinetide is to be administered to the subject after completion of treatment Period C.

Embodiment 45. The trofinetide for use of any one of Embodiments 31-44, wherein the subject experiences no treatment-emergent adverse events.

Embodiment 46. The trofinetide for use of any one of Embodiments 31-45, wherein trofinetide is to be administered to the subject in a single dose per day.

Embodiment 47. The trofinetide for use of any one of Embodiments 31-45, wherein trofinetide is to be administered to the subject in multiple doses per day that sum to the daily amount.

Embodiment 48. The trofinetide for use of Embodiment 47, wherein trofinetide is to be administered to the subject in two doses per day that sum to the daily amount.

Embodiment 49. The trofinetide for use of any one of Embodiments 31-48, wherein trofinetide is to be administered as a pharmaceutical composition comprising trofinetide and a pharmaceutically acceptable carrier.

Embodiment 50. The trofinetide for use of Embodiment 49, wherein the pharmaceutically acceptable carrier comprises water.

Embodiment 51. The trofinetide for use of Embodiment 50, wherein the pharmaceutical composition is a solution.

Embodiment 52. The trofinetide for use of Embodiment 51, wherein the concentration of trofinetide is about 0.05 g/mL-0.7 g/mL in the solution.

Embodiment 53. The trofinetide for use of Embodiment 52, wherein the concentration of trofinetide is about 0.2 g/mL in the solution.

Embodiment 54. The trofinetide for use of any one of Embodiments 31-53, wherein trofinetide is to be orally administered to the subject.

Embodiment 55. The trofinetide for use of any one of Embodiments 31-53, wherein trofinetide is to be administered by gastrostomy (G) tube to the subject.

Embodiment 56. The trofinetide for use of any one of Embodiments 31-55, wherein the subject is human.

Embodiment 57. The trofinetide for use hod of Embodiment 56, wherein the subject is female.

Embodiment 58. The trofinetide for use of any one of Embodiments 31-57, wherein the subject has a MECP2 mutation.

Embodiment 59. The trofinetide for use of any one of Embodiments 31-58, wherein the Rett syndrome is atypical Rett syndrome.

Embodiment 60. The trofinetide for use of any one of Embodiments 31-58, wherein the Rett syndrome is classic/typical Rett syndrome.

Embodiment 61. Use of Trofinetide for the manufacture of a medicament for treating Rett syndrome in a subject, wherein:

(i) a total daily dose of about 1 g to about 4 g of trofinetide is to be administered to the subject for the first 7±3 days of treatment Period A;

(ii) a total daily dose of about 4 g to about 6 g of trofinetide is to be administered to the subject for the next 14±3 days of treatment Period B;

(iii) a total daily dose of about 6 g to about 8 g of trofinetide is to be administered to the subject for the next 28±3 days of treatment Period C; and

(iv) a total daily dose of about 8 g to about 10 g of trofinetide is to be administered to the subject after completion of treatment Period C,

wherein the subject:

(a) is less than 4 years old at the time treatment Period A begins; and/or

(b) weighs between about 9 kg to about 12 kg at the time treatment Period A begins.

Embodiment 62. The use of Embodiment 61, wherein a total daily dose of about 1 g of trofinetide is to be administered to the subject for the 7±3 days of treatment Period A.

Embodiment 63. The use of Embodiment 61, wherein a total daily dose of about 2 g of trofinetide is to be administered to the subject for the 7±3 days of treatment Period A.

Embodiment 64. The use of Embodiment 61, wherein a total daily dose of about 3 g of trofinetide is to be administered to the subject for the 7±3 days of treatment Period A.

Embodiment 65. The use of Embodiment 61, wherein a total daily dose of about 4 g of trofinetide is to be administered to the subject for the 7±3 days of treatment Period A.

Embodiment 66. The use of any one of Embodiments 61-65, wherein a total daily dose of about 4 g of trofinetide is to be administered to the subject for the 14±3 days of treatment Period B.

Embodiment 67. The use of any one of Embodiments 61-65, wherein a total daily dose of about 5 g of trofinetide is to be administered to the subject for the 14±3 days of treatment Period B.

Embodiment 68. The use of any one of Embodiments 61-65, wherein a total daily dose of about 6 g of trofinetide is to be administered to the subject for the 14±3 days of treatment Period B.

Embodiment 69. The use of any one of Embodiments 61-68, wherein a total daily dose of about 6 g of trofinetide is to be administered to the subject for the 28±3 days of treatment Period C.

Embodiment 70. The use of any one of Embodiments 61-68, wherein about 7 g of trofinetide is to be administered to the subject for the 28±3 days of treatment Period C.

Embodiment 71. The use of any one of Embodiments 61-68, wherein a total daily dose of about 8 g of trofinetide is to be administered to the subject for the 28±3 days of treatment Period C.

Embodiment 72. The use of any one of Embodiments 61-71, wherein a total daily dose of about 8 g of trofinetide is to be administered to the subject after completion of treatment Period C.

Embodiment 73. The use of any one of Embodiments 61-71, wherein a total daily dose of about 9 g of trofinetide is to be administered to the subject after completion of treatment Period C.

Embodiment 74. The use of any one of Embodiments 61-71, wherein a total daily dose of about 10 g of trofinetide is to be administered to the subject after completion of treatment Period C.

Embodiment 75. The use of any one of Embodiments 61-74, wherein the subject experiences no treatment-emergent adverse events.

Embodiment 76. The use of any one of Embodiments 61-75, wherein trofinetide is to be administered to the subject in a single dose per day.

Embodiment 77. The use of any one of Embodiments 61-75, wherein trofinetide is to be administered to the subject in multiple doses per day that sum to the daily amount.

Embodiment 78. The use of Embodiment 77, wherein trofinetide is to be administered to the subject in two doses per day that sum to the daily amount.

Embodiment 79. The use of any one of Embodiments 61-78, wherein trofinetide is to be administered as a pharmaceutical composition comprising trofinetide and a pharmaceutically acceptable carrier.

Embodiment 80. The use of Embodiment 79, wherein the pharmaceutically acceptable carrier comprises water.

Embodiment 81. The use of Embodiment 80, wherein the pharmaceutical composition is a solution.

Embodiment 82. The use of Embodiment 81, wherein the concentration of trofinetide is about 0.05 g/mL-0.7 g/mL in the solution.

Embodiment 83. The use of Embodiment 52, wherein the concentration of trofinetide is about 0.2 g/mL in the solution.

Embodiment 84. The use of any one of Embodiments 61-83, wherein trofinetide is to be orally administered to the subject.

Embodiment 85. The use of any one of Embodiments 61-83, wherein trofinetide is to be administered by gastrostomy (G) tube to the subject.

Embodiment 86. The use of any one of Embodiments 61-85, wherein the subject is human.

Embodiment 87. The use of Embodiment 86, wherein the subject is female.

Embodiment 88. The use of any one of Embodiments 61-87, wherein the subject has a MECP2 mutation.

Embodiment 89. The use of any one of Embodiments 61-88, wherein the Rett syndrome is atypical Rett syndrome.

Embodiment 90. The use of any one of Embodiments 61-88, wherein the Rett syndrome is classic/typical Rett syndrome.

Embodiment 91. The method of any one of Embodiments 1-30, wherein the total daily dose of trofinetide administered to the subject after completion of treatment Period A, treatment Period B, or treatment Period C results in an AUC_{0-12, ss} of about 755 μg·h/mL to about 1300 μg·h/mL.

Embodiment 92. The method of Embodiment 91, wherein the total daily dose of trofinetide administered to the subject after completion of treatment Period C results in an AUC_{0-12, ss} of about 800 μg·h/mL to about 1000 μg·h/mL.

Embodiment 93. The method of any one of Embodiments 1-30, wherein the total daily dose of trofinetide administered to the subject after completion of treatment Period A, treatment Period B, or treatment Period C results in an AUC_{0-12, ss} of at least about 755 μg·h/mL, at least about 780 μg·h/mL, at least about 790 μg·h/mL, at least about 800 μg·h/mL, at least about 820 μg·h/mL, at least about 840 μg·h/mL, at least about 860 μg·h/mL, at least about 880 μg·h/mL, at least about 900 μg·h/mL, at least about 920 μg·h/mL, at least about 940 μg·h/mL, at least about 960 μg·h/mL, at least about 980 μg·h/mL, at least about 1000 μg·h/mL, at least about 1020 μg·h/mL, at least about 1040 μg·h/mL, at least about 1060 μg·h/mL, at least about 1080 μg·h/mL, at least about 1100 μg·h/mL, at least about 1120 μg·h/mL, at least about 1140 μg·h/mL, at least about 1160 μg·h/mL, at least about 1180 μg·h/mL, at least about 1200 μg·h/mL, at least about 1220 μg·h/mL, at least about 1240 μg·h/mL, at least about 1260 μg·h/mL, or at least about 1280 μg·h/mL in the subject.

Embodiment 94. The trofinetide for use of any one of Embodiments 31-60, wherein the total daily dose of trofinetide administered to the subject after completion of treatment Period A, treatment Period B, or treatment Period C results in an AUC_{0-12, ss} of about 755 μg·h/mL to about 1300 μg·h/mL.

Embodiment 95. The trofinetide for use of Embodiment 94, wherein the total daily dose of trofinetide administered to the subject after completion of treatment Period C results in an AUC_{0-12, ss} of about 800 μg·h/mL to about 1000 μg·h/mL.

Embodiment 96. The trofinetide for use of any one of Embodiments 31-60, wherein the total daily dose of trofinetide administered to the subject after completion of treatment Period A, treatment Period B, or treatment Period C results in an AUC_{0-12, ss} of at least about 755 μg·h/mL, at least about 780 μg·h/mL, at least about 790 μg·h/mL, at least about 800 μg·h/mL, at least about 820 μg·h/mL, at least about 840 μg·h/mL, at least about 860 μg·h/mL, at least about 880 μg·h/mL, at least about 900 μg·h/mL, at least about 920 μg·h/mL, at least about 940 μg·h/mL, at least about 960 μg·h/mL, at least about 980 μg·h/mL, at least about 1000 μg·h/mL, at least about 1020 μg·h/mL, at least about 1040 μg·h/mL, at least about 1060 μg·h/mL, at least about 1080 μg·h/mL, at least about 1100 μg·h/mL, at least about 1120 μg·h/mL, at least about 1140 μg·h/mL, at least about 1160 μg·h/mL, at least about 1180 μg·h/mL, at least about 1200 μg·h/mL, at least about 1220 μg·h/mL, at least about 1240 μg·h/mL, at least about 1260 μg·h/mL, or at least about 1280 μg·h/mL in the subject.

Embodiment 97. The use of any one of Embodiments 61-90, wherein the total daily dose of trofinetide administered to the subject after completion of treatment Period A, treatment Period B, or treatment Period C results in an AUC_{0-12, ss} of about 755 μg·h/mL to about 1300 μg·h/mL.

Embodiment 98. The trofinetide for use of Embodiment 97, wherein the total daily dose of trofinetide administered to the subject after completion of treatment Period C results in an AUC_{0-12, ss} of about 800 μg·h/mL to about 1000 μg·h/mL.

Embodiment 99. The trofinetide for use of any one of Embodiments 61-90, wherein the total daily dose of trofinetide administered to the subject after completion of treatment Period A, treatment Period B, or treatment Period C results in an AUC_{0-12, ss} of at least about 755 μg·h/mL, at least about 780 μg·h/mL, at least about 790 μg·h/mL, at least about 800 μg·h/mL, at least about 820 μg·h/mL, at least about 840 μg·h/mL, at least about 860 μg·h/mL, at least about 880 μg·h/mL, at least about 900 μg·h/mL, at least about 920 μg·h/mL, at least about 940 μg·h/mL, at least about 960 μg·h/mL, at least about 980 μg·h/mL, at least about 1000 μg·h/mL, at least about 1020 μg·h/mL, at least about 1040 μg·h/mL, at least about 1060 μg·h/mL, at least about 1080 μg·h/mL, at least about 1100 μg·h/mL, at least about 1120 μg·h/mL, at least about 1140 μg·h/mL, at least about 1160 μg·h/mL, at least about 1180 μg·h/mL, at least about 1200 μg·h/mL, at least about 1220 μg·h/mL, at least about 1240 μg·h/mL, at least about 1260 μg·h/mL, or at least about 1280 μg·h/mL in the subject.

Embodiment 100. The method of any one of Embodiments 1-30, wherein the total daily dose of trofinetide administered to the subject after completion of treatment Period A, treatment Period B, or treatment Period C results in a C_{max, ss} of about 100 μg/mL to about 300 μg/mL in the subject.

Embodiment 101. The method of Embodiment 100, wherein the total daily dose of trofinetide administered to the subject after completion of treatment Period C results in a C_{max, ss} of about 100 μg/mL to about 200 μg/mL in the subject.

Embodiment 102. The method of any one of Embodiments 1-30, wherein the total daily dose of trofinetide administered to the subject after completion of treatment Period A, treatment Period B, or treatment Period C results in a C_{max, ss} at least about 100 μg/mL, at least about 110 μg/mL, at least about 120 μg/mL, at least about 130 μg/mL, at least about 140 μg/mL, at least about 150 μg/mL, at least about 160 μg/mL, at least about 170 μg/mL, at least about 180 μg/mL, at least about 190 μg/mL, or at least about 200 μg/mL in the subject.

Embodiment 103. The trofinetide for use of any one of Embodiments 31-60, wherein the total daily dose of trofinetide administered to the subject after completion of treatment Period A, treatment Period B, or treatment Period C results in a C_{max, ss} of about 100 μg/mL to about 300 μg/mL in the subject.

Embodiment 104. The trofinetide for use of Embodiment 103, wherein the total daily dose of trofinetide administered to the subject after completion of treatment Period C results in a C_{max, ss} of about 100 μg/mL to about 200 μg/mL in the subject.

Embodiment 105. The trofinetide for use of any one of Embodiments 31-60, wherein the total daily dose of trofinetide administered to the subject after completion of treatment Period A, treatment Period B, or treatment Period C results in a C_{max, ss} at least about 100 μg/mL, at least about 110 μg/mL, at least about 120 μg/mL, at least about 130 μg/mL, at least about 140 μg/mL, at least about 150 μg/mL, at least about 160 μg/mL, at least about 170 μg/mL, at least about 180 μg/mL, at least about 190 μg/mL, or at least about 200 μg/mL in the subject.

Embodiment 106. The use of any one of Embodiments 61-90, wherein the total daily dose of trofinetide administered to the subject after completion of treatment Period A, treatment Period B, or treatment Period C results in a C_{max, ss} of about 100 μg/mL to about 300 μg/mL in the subject.

Embodiment 107. The use of Embodiment 106, wherein the total daily dose of trofinetide administered to the subject after completion of treatment Period C results in a C_{max, ss} of about 100 μg/mL to about 200 μg/mL in the subject.

Embodiment 108. The use of any one of Embodiments 61-90, wherein the total daily dose of trofinetide administered to the subject after completion of treatment Period A, treatment Period B, or treatment Period C results in a C_{max, ss} at least about 100 μg/mL, at least about 110 μg/mL, at least about 120 μg/mL, at least about 130 μg/mL, at least about 140 μg/mL, at least about 150 μg/mL, at least about 160 μg/mL, at least about 170 μg/mL, at least about 180 μg/mL, at least about 190 μg/mL, or at least about 200 μg/mL in the subject.

Embodiment 109. The method of Embodiment 93, wherein the administration of trofinetide results in an AUC_{0-12, ss} of at least 755 μg·h/mL, such as 755 μg·h/mL to 1300 μg·h/mL, such as 780 μg·h/mL to 1300 μg·h/mL, such as 790 μg·h/mL to 1300 μg·h/mL, such as 800 μg·h/mL to 1300 μg·h/mL, such as 820 μg·h/mL to 1300 μg·h/mL, such as 840 μg·h/mL to 1300 μg·h/mL, such as 860 μg·h/mL to 1300 μg·h/mL, such as 880 μg·h/mL to 1300 μg·h/mL, such as 900 μg·h/mL to 1300 μg·h/mL, such as 920 μg·h/mL to 1300 μg·h/mL, such as 940 μg·h/mL to 1300 μg·h/mL, such as 960 μg·h/mL to 1300 μg·h/mL, such as 980 μg·h/mL to 1300 μg·h/mL, such as 1000 μg·h/mL to 1300 μg·h/mL, such as 1020 μg·h/mL to 1300 μg·h/mL, such as 1040 μg·h/mL to 1300 μg·h/mL, such as 1060 μg·h/mL to 1300 μg·h/mL, such as 1080 μg·h/mL to 1300 μg·h/mL, such as 1100 μg·h/mL to 1300 μg·h/mL, such as 1120 μg·h/mL to 1300 μg·h/mL, such as 1140 μg·h/mL to 1300 μg·h/mL, such as 1160 μg·h/mL to 1300 μg·h/mL, such as 1180 μg·h/mL to 1300 μg·h/mL, such as 1200 μg·h/mL to 1300 μg·h/mL, such as 1220 μg·h/mL to 1300 μg·h/mL, such as 1240 μg·h/mL to 1300 μg·h/mL, such as 1260 μg·h/mL to 1300 μg·h/mL, or such as 1280 μg·h/mL to 1300 μg·h/mL, in the subject.

Embodiment 110. The use of Embodiment 109, wherein the administration of trofinetide results in an AUC_{0-12, ss} of 755 μg·h/mL to 1300 μg·h/mL in the subject.

Embodiment 111. The use of Embodiment 110, wherein the administration of trofinetide results in an AUC_{0-12, ss} of 780 μg·h/mL to 1300 μg·h/mL in the subject.

Embodiment 112. The use of Embodiment 111, wherein the administration of trofinetide results in an AUC_{0-12, ss} of 790 μg·h/mL to 1300 μg·h/mL in the subject.

Embodiment 113. The trofinetide for use of Embodiment 96, wherein the administration of trofinetide results in an AUC_{0-12, ss} of at least 755 μg·h/mL, such as 755 μg·h/mL to 1300 μg·h/mL, such as 780 μg·h/mL to 1300 μg·h/mL, such as 790 μg·h/mL to 1300 μg·h/mL, such as 800 μg·h/mL to 1300 μg·h/mL, such as 820 μg·h/mL to 1300 μg·h/mL, such as 840 μg·h/mL to 1300 μg·h/mL, such as 860 μg·h/mL to 1300 μg·h/mL, such as 880 μg·h/mL to 1300 μg·h/mL, such as 900 μg·h/mL to 1300 μg·h/mL, such as 920 μg·h/mL to 1300 μg·h/mL, such as 940 μg·h/mL to 1300 μg·h/mL, such as 960 μg·h/mL to 1300 μg·h/mL, such as 980 μg·h/mL to 1300 μg·h/mL, such as 1000 μg·h/mL to 1300 μg·h/mL, such as 1020 μg·h/mL to 1300 μg·h/mL, such as 1040 μg·h/mL to 1300 μg·h/mL, such as 1060 μg·h/mL to 1300 μg·h/mL, such as 1080 μg·h/mL to 1300 μg·h/mL, such as 1100 μg·h/mL to 1300 μg·h/mL, such as 1120 μg·h/mL to 1300 μg·h/mL, such as 1140 μg·h/mL to 1300 μg·h/mL, such as 1160 μg·h/mL to 1300 μg·h/mL, such as 1180 μg·h/mL to 1300 μg·h/mL, such as 1200 μg·h/mL to 1300 μg·h/mL, such as 1220 μg·h/mL to 1300 μg·h/mL, such as 1240 μg·h/mL to 1300 μg·h/mL, such as 1260 μg·h/mL to 1300 μg·h/mL, or such as 1280 μg·h/mL to 1300 μg·h/mL, in the subject.

Embodiment 114. The trofinetide for use of Embodiment 113, wherein the administration of trofinetide results in an AUC_{0-12, ss} of 755 μg·h/mL to 1300 μg·h/mL in the subject.

Embodiment 115. The trofinetide for use of Embodiment 114, wherein the administration of trofinetide results in an AUC_{0-12, ss} of 780 μg·h/mL to 1300 μg·h/mL in the subject.

Embodiment 116. The trofinetide for use of Embodiment 115, wherein the administration of trofinetide results in an AUC_{0-12, ss} of 790 μg·h/mL to 1300 μg·h/mL in the subject.

Embodiment 117. The use of Embodiment 99, wherein the administration of trofinetide results in an AUC_{0-12, ss} of at least 755 μg·h/mL, such as 755 μg·h/mL to 1300 μg·h/mL, such as 780 μg·h/mL to 1300 μg·h/mL, such as 790 μg·h/mL to 1300 μg·h/mL, such as 800 μg·h/mL to 1300 μg·h/mL, such as 820 μg·h/mL to 1300 μg·h/mL, such as 840 μg·h/mL to 1300 μg·h/mL, such as 860 μg·h/mL to 1300 μg·h/mL, such as 880 μg·h/mL to 1300 μg·h/mL, such as 900 μg·h/mL to 1300 μg·h/mL, such as 920 μg·h/mL to 1300 μg·h/mL, such as 940 μg·h/mL to 1300 μg·h/mL, such as 960 μg·h/mL to 1300 μg·h/mL, such as 980 μg·h/mL to 1300 μg·h/mL, such as 1000 μg·h/mL to 1300 μg·h/mL, such as 1020 μg·h/mL to 1300 μg·h/mL, such as 1040 μg·h/mL to 1300 μg·h/mL, such as 1060 μg·h/mL to 1300 μg·h/mL, such as 1080 μg·h/mL to 1300 μg·h/mL, such as 1100 μg·h/mL to 1300 μg·h/mL, such as 1120 μg·h/mL to 1300 μg·h/mL, such as 1140 μg·h/mL to 1300 μg·h/mL, such as 1160 μg·h/mL to 1300 μg·h/mL, such as 1180 μg·h/mL to 1300 μg·h/mL, such as 1200 μg·h/mL to 1300 μg·h/mL, such as 1220 μg·h/mL to 1300 μg·h/mL, such as 1240 μg·h/mL to 1300 μg·h/mL, such as 1260 μg·h/mL to 1300 μg·h/mL, or such as 1280 μg·h/mL to 1300 μg·h/mL, in the subject.

Embodiment 118. The use of Embodiment 117, wherein the administration of trofinetide results in an AUC_{0-12, ss} of 755 μg·h/mL to 1300 μg·h/mL in the subject.

Embodiment 119. The use of Embodiment 118, wherein the administration of trofinetide results in an AUC_{0-12, ss} of 780 μg·h/mL to 1300 μg·h/mL in the subject.

Embodiment 120. The use of Embodiment 119, wherein the administration of trofinetide results in an AUC_{0-12, ss} of 790 μg·h/mL to 1300 μg·h/mL in the subject.

Embodiment 121. The method Embodiment 102, wherein the total daily dose of trofinetide administered to the subject after completion of treatment Period A, treatment Period B, or treatment Period C results in a C_{max, ss} of at least 100 μg/mL in the subject, such as at 100 μg/mL to 300 μg/mL, such as 110 μg/mL to 300 μg/mL, such as 120 μg/mL to 300 μg/mL, such as 130 μg/mL to 300 μg/mL, such as 140 μg/mL to 300 μg/mL, such as 150 μg/mL to 300 μg/mL, such as 160 μg/mL to 300 μg/mL, such as 170 μg/mL to 300 μg/mL, such as 180 μg/mL to 300 μg/mL, such as 190 μg/mL to 300 μg/mL, such as 200 μg/mL to 300 μg/mL, such as 210 μg/mL to 300 μg/mL, such as 220 μg/mL to 300 μg/mL, such as 230 μg/mL to 300 μg/mL, such as 240 μg/mL to 300 g/mL, such as 250 g/mL to 300 g/mL, such as 260 g/mL to 300 μg/mL, such as 270 μg/mL to 300 μg/mL, such as 280 μg/mL to 300 μg/mL, or such as 290 μg/mL to 300 μg/mL, in the subject.

Embodiment 122. The trofinetide for use of Embodiment 105, wherein the total daily dose of trofinetide administered to the subject after completion of treatment Period A, treatment Period B, or treatment Period C results in a C_{max, ss} of at least 100 μg/mL in the subject, such as at 100 μg/mL to 300 μg/mL, such as 110 μg/mL to 300 μg/mL, such as 120 μg/mL to 300 μg/mL, such as 130 μg/mL to 300 μg/mL, such as 140 μg/mL to 300 μg/mL, such as 150 μg/mL to 300 μg/mL, such as 160 μg/mL to 300 μg/mL, such as 170 μg/mL to 300 μg/mL, such as 180 μg/mL to 300 μg/mL, such as 190 μg/mL to 300 μg/mL, such as 200 μg/mL to 300 μg/mL, such as 210 μg/mL to 300 μg/mL, such as 220 μg/mL to 300 μg/mL, such as 230 μg/mL to 300 μg/mL, such as 240 μg/mL to 300 μg/mL, such as 250 μg/mL to 300 μg/mL, such as 260 μg/mL to 300 μg/mL, such as 270 μg/mL to 300 μg/mL, such as 280 μg/mL to 300 μg/mL, or such as 290 μg/mL to 300 μg/mL, in the subject

Embodiment 123. The use of Embodiment 108, wherein the total daily dose of trofinetide administered to the subject after completion of treatment Period A, treatment Period B, or treatment Period C results in a C_{max, ss} of at least 100 μg/mL in the subject, such as at 100 μg/mL to 300 μg/mL, such as 110 μg/mL to 300 μg/mL, such as 120 μg/mL to 300 μg/mL, such as 130 μg/mL to 300 μg/mL, such as 140 μg/mL to 300 μg/mL, such as 150 μg/mL to 300 μg/mL, such as 160 μg/mL to 300 μg/mL, such as 170 μg/mL to 300 μg/mL, such as 180 μg/mL to 300 μg/mL, such as 190 μg/mL to 300 μg/mL, such as 200 μg/mL to 300 μg/mL, such as 210 μg/mL to 300 μg/mL, such as 220 μg/mL to 300 μg/mL, such as 230 μg/mL to 300 μg/mL, such as 240 μg/mL to 300 μg/mL, such as 250 μg/mL to 300 μg/mL, such as 260 μg/mL to 300 μg/mL, such as 270 μg/mL to 300 μg/mL, such as 280 μg/mL to 300 μg/mL, or such as 290 μg/mL to 300 μg/mL, in the subject.

Subjects with Rett syndrome (unless otherwise indicated, this term is used in the broadest sense, including Rett-like syndrome and variants of Rett syndrome) have been found to have mutations in any of a number of genes, including MECP2, ACTL6B, AGAP6, ANKRD31, ARHGEF10L, ATP8B1, BTBD9, CACNA1I, CDKL5, CHD4, CHRNA5, CTNNB1, EEF1A2, EIF2B2, EIF4G1, FAM151A, FAT3, FOXG1, GABBR2, GABRD, GRAMD1A, GRIN1, GRIN2B, HAP1, HCN1, HDAC1, HTT, IMPDH2, IQGAP3, IQSEC2, IZUMO4, JMJD1C, KCNA2, KCNJ10, KCNQ2, KIF1A, LAMB2, LRRC40, MEF2C, MGRN1, NCOR2, PDLIM7, PPT1, PWP2, RHOBTB2, SAFB2, SATB2, SCG2, SCN1A, SCN2A, SCN8A, SDHA, SEMA6B, SHANK3, SHROOM4, SLC2A1, SLC35A2, SLC39A13, SLC6A1, SMARCA1, SMC1A, SRRM3, STXBP1, SYNE2, SYNGAP1, TAF1B, TBLXR1, TCF4, TRRAP, VASH2, WDR45, X4B2, ZFX, ZNF238, ZNF620, and ZSCAN12. See, e.g., Ehrhart, F. et al., “Current Developments in the Genetics of Rett and Rett-Like Syndrome,” Curr Opin Psychiatry 31, no. 2 (March 2018): 103-08, dx.doi.org/10.1097/YCO.0000000000000389; Iwama, K., et al., “Genetic Landscape of Rett Syndrome-Like Phenotypes Revealed by Whole Exome Sequencing,” J Med Genet (Mar. 6 2019); jmedgenet-2018-105775, dx.doi.org/10.1136/jmedgenet-2018-105775; and Wang, J., et al., “Rett and Rett-Like Syndrome: Expanding the Genetic Spectrum to Kif1a and Grin1 Gene,” Mol Genet Genomic Med (Sep. 11, 2019): e968, dx.doi.org/10.1002/mgg3.968. Accordingly, in some embodiments, the subject has a mutation associated with Rett syndrome, e.g., which is in any of the genes listed above. In some embodiments, the subject has a MECP2 mutation. In some embodiments, the subject has a MECP2 mutation selected from a nonsense mutation, a missense mutation, a C-terminal truncation, a deletion, R168X, R270X, R255X, T158M, R306C, and R106W. In some embodiments, the subject has a MECP2 mutation selected from R168X, R270X, R255X, T158M, and R306C. In some embodiments, the subject has a MECP2 mutation selected from R168X, R270X, R255X, and T158M. In some embodiments, the subject has a MECP2 mutation selected from R168X, R270X, and R255X. In some embodiments, the subject has a MECP2 mutation selected from R168X and R270X. In some embodiments, the subject has a R168X MECP2 mutation. In some embodiments, the subject has a mutation in ACTL6B, AGAP6, ANKRD31, ARHGEF10L, ATP8B1, BTBD9, CACNA1I, CDKL5, CHD4, CHRNA5, CTNNB1, EFF1A2, EIF2B2, EIF4G1, FAM151A, FAT3, FOXG1, GABBR2, GABRD, GRAMD1A, GRIN1, GRIN2B, HAP1, HCN1, HDAC1, HTT, IMPDH2, IQGAP3, IQSEC2, IZUMO4, JMJD1C, KCNA2, KCNJ10, KCNQ2, KIF1A, LAMB2, LRRC40, MEF2C, MGRN1, NCOR2, PDLIM7, PPT1, PWP2, RHOBTB2, SAFB2, SATB2, SCG2, SCN1A, SCN2A, SCN8A, SDHA, SEMA6B, SHANK3, SHROOM4, SLC2A1, SLC35A2, SLC39A13, SLC6A1, SMARCA1, SMC1A, SRRM3, STXBP1, SYNE2, SYNGAP1, TAF1B, TBLXR1, TCF4, TRRAP, VASH2, WDR45, XAB2, ZFX, ZNF238, ZNF620, or ZSCAN12.

As used herein, a “subject in need thereof,” in the context of a method of treating Rett syndrome, comprising administering trofinetide to a subject in need thereof, is a subject with classic/typical Rett syndrome, atypical Rett syndrome, “possible,” “possibly,” “probable,” or “probably” Rett syndrome, “possible,” “possibly,” “probable,” or “probably” atypical Rett syndrome, “Rett-like syndrome,” one or more symptoms of Rett syndrome, a mutation associated with Rett syndrome (e.g., any of the mutations discussed above, or a MECP2 mutation that is a nonsense mutation, a C-terminal truncation, a deletion, R168X, R270X, R255X, T158M, R306C, or R106W), and/or a mutation that has been observed in subjects with Rett syndrome.

The term “therapeutically effective amount,” as used herein, refers to that amount of trofinetide sufficient to result in amelioration of one or more symptoms of Rett Syndrome, or prevent advancement of Rett Syndrome, or cause regression of Rett Syndrome in a subject in need thereof. For example, a therapeutically effective amount refers to the amount of trofinetide that causes a therapeutic response, e.g., delay the progression of Rett Syndrome in subject by at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%, or more.

The term “about,” as used herein, includes the recited number±10%. Thus, “about 10” means 9 to 11.

In some embodiments, the Rett syndrome is atypical or classic/typical Rett syndrome. In some embodiments, the Rett syndrome is classic/typical Rett syndrome. In some embodiments, the Rett syndrome is “possible” or “possibly” Rett syndrome. In some embodiments, the Rett syndrome is “possible” or “possibly” atypical Rett syndrome. In some embodiments, the Rett syndrome is “probable” or “probably” Rett syndrome. In some embodiments, the Rett syndrome is “probable” or “probably” atypical Rett syndrome.

In some embodiments, a subject with classic/typical Rett syndrome displays all of the following clinical parameters: partial or complete loss of acquired purposeful hand skills; partial or complete loss of acquired spoken language; gait abnormalities (e.g., impaired (dyspraxic) gait or absence of ability); and stereotypic hand movements (e.g., hand wringing/squeezing, clapping/tapping, mouthing, and washing/rubbing automatisms). Loss of acquired language is based on best acquired spoken language skill, not strictly on the acquisition of distinct words or higher language skills. Thus, an individual who had learned to babble but then loses this ability is considered to have a loss of acquired language.

In some embodiments, a subject with classic/typical Rett syndrome optionally displays one or more supporting criteria selected from: breathing disturbances when awake; bruxism when awake; impaired sleep pattern; abnormal muscle tone; peripheral vasomotor disturbances; scoliosis/kyphosis; growth retardation; small, cold hands and feet; inappropriate laughing/or screaming spells; diminished response to pain; and intense eye communication (e.g., eye pointing).

In some embodiments, a subject with classic/typical Rett syndrome has not had an insult that causes neurological problems (e.g., supported by clinical evidence, such as a neurological or ophthalmological examination and MRI/CT, that the presumed insult directly resulted in neurological dysfunction) or grossly abnormal psychomotor development in the first six months of life. In some embodiments, the insult is brain injury secondary to trauma (e.g., peri- or postnatally), neurometabolic disease, or severe infection. Grossly abnormal means abnormal to the point that normal milestones (acquiring head control, swallowing, developing social smile) were not met. Mild generalized hypotonia or other previously reported subtle developmental alterations during the first six months of life is common in RTT and do not constitute grossly abnormal psychomotor development in the first six months of life.

In some embodiments, a subject with atypical Rett syndrome displays:
1) two or three clinical parameters selected from: partial or complete loss of acquired purposeful hand skills; partial or complete loss of acquired spoken language; gait abnormalities (e.g., impaired (dyspraxic) gait or absence of ability); and stereotypic hand movements (e.g., hand wringing/squeezing, clapping/tapping, mouthing, and washing/rubbing automatisms); and
2) at least five supporting criteria selected from: breathing disturbances when awake; bruxism when awake; impaired sleep pattern; abnormal muscle tone; peripheral vasomotor disturbances; scoliosis/kyphosis; growth retardation; small, cold hands and feet; inappropriate laughing/or screaming spells; diminished response to pain; and intense eye communication (e.g., eye pointing).

If an individual has or ever had a clinical feature listed it is counted as a supportive criterion. Many of these features have an age dependency, manifesting and becoming more predominant at certain ages. Therefore, the diagnosis of atypical RTT may be easier for older individuals than for younger.

In some embodiments, a subject with atypical Rett syndrome is a younger individual (under 5 years old, e.g., 18 months to 5 years old) who has a period of regression and at least two clinical parameters but does not fulfill the requirement of at least five supportive criteria, the diagnosis of “probably atypical RTT” may be given. Individuals who fall into this category should be reassessed as they age and the diagnosis revised accordingly.

In some embodiments, a subject with Rett syndrome is an individual under 3 years old who has an MECP2 mutation and has not lost any skills (e.g., prior to any clear evidence of regression) but otherwise has clinical features suggestive of Rett syndrome, e.g., one or more of gait abnormalities (e.g., impaired (dyspraxic) gait or absence of ability); and stereotypic hand movements (e.g., hand wringing/squeezing, clapping/tapping, mouthing, and washing/rubbing automatisms); breathing disturbances when awake; bruxism when awake; impaired sleep pattern; abnormal muscle tone; peripheral vasomotor disturbances; scoliosis/kyphosis; growth retardation; small, cold hands and feet; inappropriate laughing/or screaming spells; diminished response to pain; and intense eye communication (e.g., eye pointing). In such cases, the Rett syndrome is “possible” atypical Rett syndrome. Such subjects should be reassessed every six to 12 months for evidence of regression.

In some embodiments, a subject with Rett syndrome is in a period of regression followed by recovery or stabilization.

In some embodiments, a subject with Rett-like syndrome does not completely fulfill the criteria for atypical Rett syndrome or typical Rett syndrome but shows some clinical features of RTT, such as any combination of clinical features discussed above; for example, two, three, or four of psychomotor retardation with or without regression, stereotypic hand movements, loss of hand use, and poor language.

### EXAMPLES

### Example 1: Pharmacokinetic Analysis of Trofinetide from Phase 2 Studies

In the prior Rett syndrome Phase 2 studies (Glaze el al., 2019), trofinetide showed linear pharmacokinetics across the dose range tested in pediatric RTT patients. These pharmacokinetic results are in agreement with the data obtained previously in healthy subjects and in adolescent and adult RTT patients. From a drug metabolism perspective, there was no accumulation, metabolic inhibition, or induction observed during treatment. For subjects treated with 50 mg/kg BID, median Cₘₐₓ was 17.7 μg/mL and median AUC(₀₋₁₂ₛₛ) was 139.4 μg/mL·h. For subjects treated with 100 mg/kg BID, median Cₘₐₓ was 52.6 μg/mL and median AUC(₀₋₁₂ₛₛ) was 338.6 μg/mL·h. For subjects treated with 200 mg/kg BID, median Cₘₐₓ was 82.2 μg/mL and median AUC(₀₋₁₂ₛₛ) was 505.1 μg/mL·h. The geometric mean of the apparent terminal elimination half-life (T_{1/2}) varied from 5.3 hr to 6.1 hr across the three dosing groups.

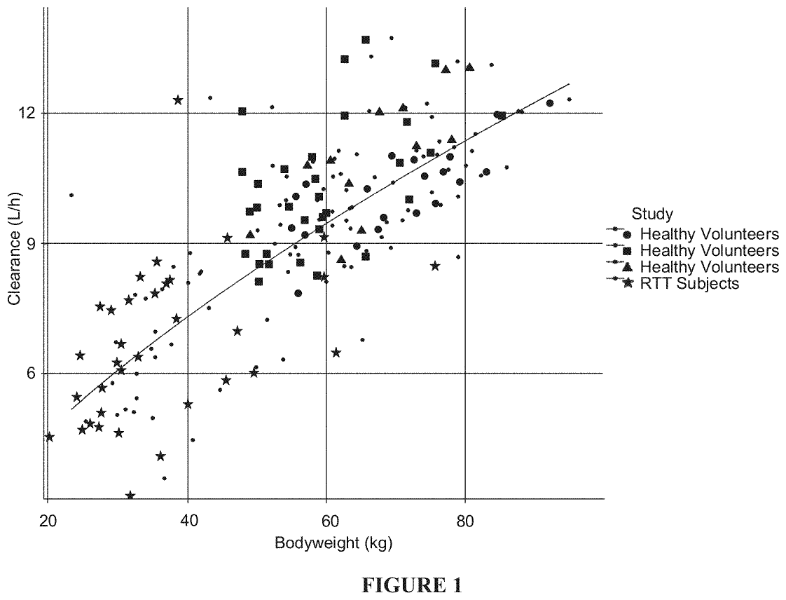

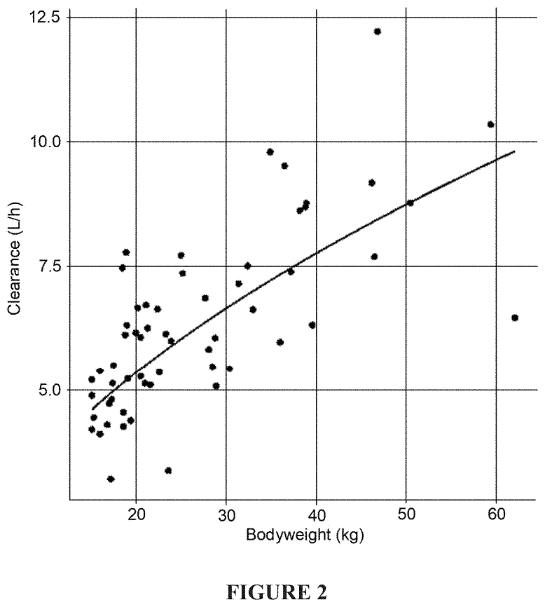

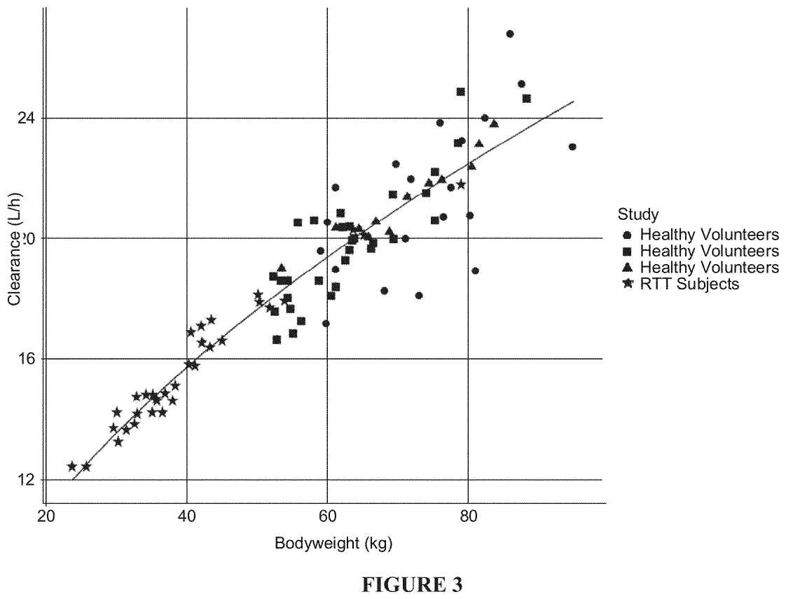

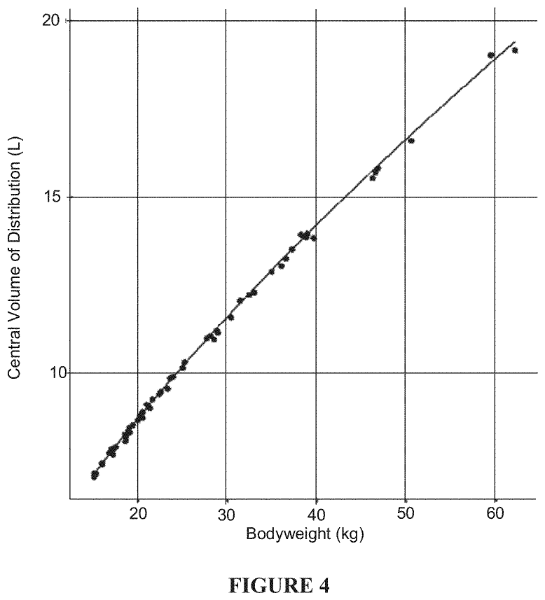

Further analysis provided by the present disclosure has now demonstrated that body weight had a significant effect on clearance, as shown in FIG. 1 (adolescents and adults vs. healthy volunteers) and FIG. 2 (children and adolescents), and on central volume of distribution, as shown in FIG. 3 (adolescents and adults vs. healthy volunteers) and FIG. 4 (children and adolescents). The range of drug exposure for subjects receiving the 200 mg/kg BID dose level in children and adolescents was below the expected exposure range for this dose level.

A model of simulated dose levels was developed to characterize the dose levels required to reach a minimal target exposure level of orally administered trofinetide at different weights in a pediatric population.

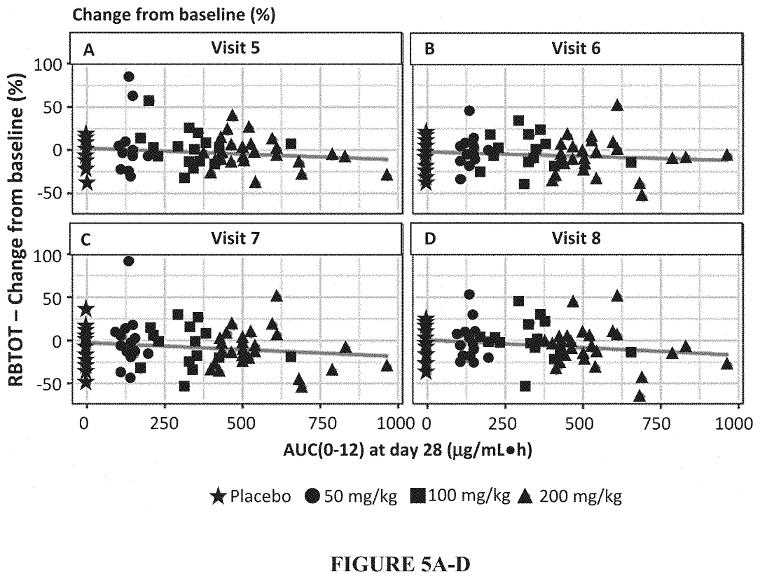

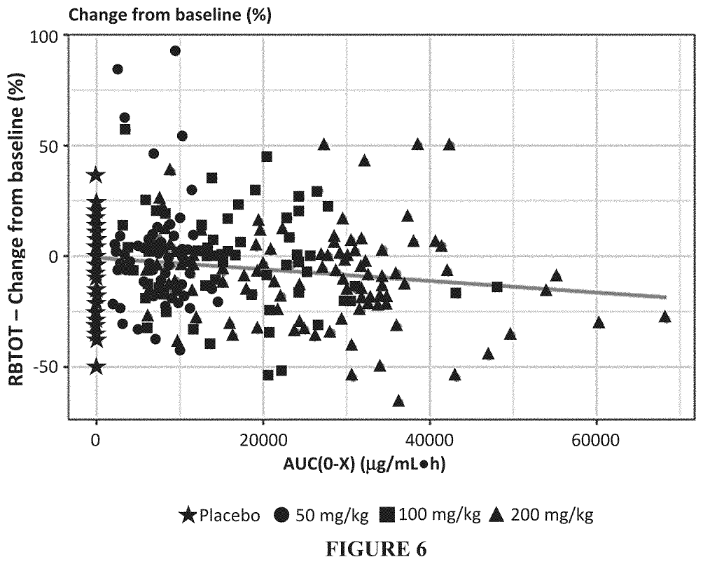

Even with a lower than expected range of exposures, the 200 mg/kg BID dose showed evidence of clinical efficacy, and exploratory analyses suggested a positive PK/PD relationship. A correlation was observed between systemic exposure and changes in RSBQ, RTT-DSC, and CGI-I, where improvements were observed with higher exposure (steady state AUC (AUCss)) and with higher cumulative exposure. For example, the results showed a correlation between percentage change from treatment baseline in RSBQ total score and trofinetide AUCss by visit (FIG. 5) and during the active dosing period (FIG. 6).

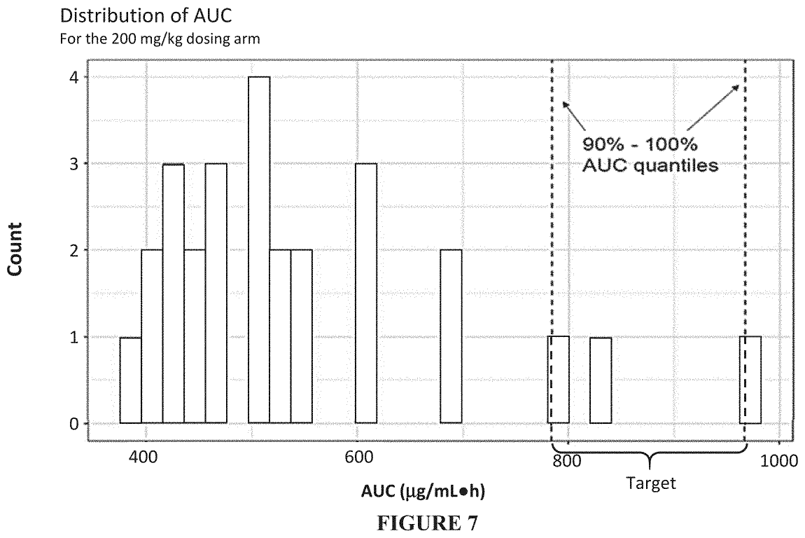

Therefore, the upper levels of exposure in the 200 mg/kg BID dose level were considered to be effective and thus an appropriate target exposure level. Thus, dosing strategies were developed with the aim of providing a minimal target exposure of 800 μg/mL·h, corresponding approximately to the 90-100% AUC quantile of exposure (AUC₀₋₁₂) observed in the children/adolescents study for the nominal dose of 200 mg/kg BID. The 90-100% quantile in that study included AUC₍₀₋₁₂₎ of 790 μg/mL·h and higher, as shown in FIG. 7. With weight-banding, a greater proportion of subjects is expected to achieve the target drug exposure.

As seen in Table 2 below, five scenarios for dosing were considered for Phase 3 studies. These scenarios each consisted of one or more subject body weight ranges, collectively covering 15 to 70 kg, with the indicated BID dose level assigned to each body weight range.

The body weight combinations were evaluated for projected exposures, as expressed by AUC₀₋₁₂, using population pharmacokinetics and simulation-based techniques to model a simulated 12-hour dosing interval. For each body weight/dose level combination, exposures reached by the 5^{th}, 25^{th}, 50^{th}, 75^{th}, and 95^{th} percentiles of subjects were projected. In some embodiments, the AUC₀₋₁₂ is measured at steady-state, e.g., after 42 days of dosing.

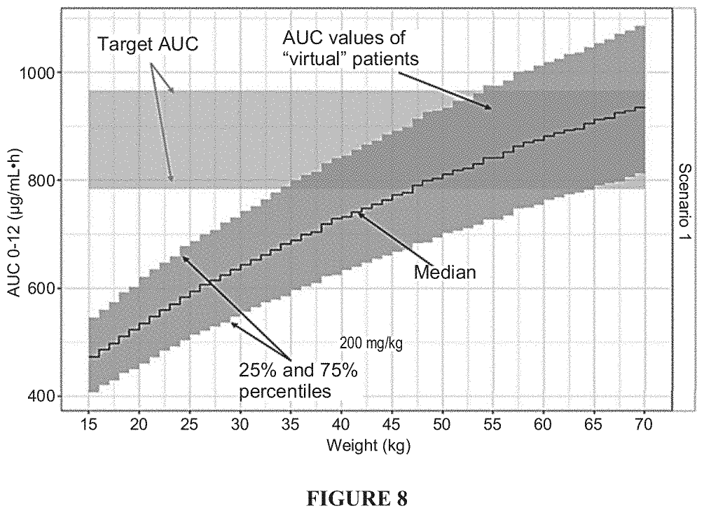

Scenario 1, using a single body weight band, was projected to leave the majority of patients under 45 kg under the target AUC₀₋₁₂ range, as shown in FIG. 8.

Scenario 4, using four body weight bands, provided overlap with the target exposure range for each body weight band and similarity of exposure across body weight bands. The 50^{th} percentile exposure was chosen for the exposure multiple calculation. In Scenario 4, the highest projected 50^{th} percentile exposure among the four body weight ranges was 882 h·ng/mL, and this value was used in the exposure multiple calculation.

TABLE 2
Five Dosing Scenarios Based on Weight Banding

| | Body | Dose | | | | | |
|---|---|---|---|---|---|---|---|
| | weight | (mg, | AUC₀₋₁₂ (h · ng/mL) by percentile | | | | |
| Scenario | (kg) | BID) | 5^{th} | 25^{th} | 50^{th} | 75^{th} | 95^{th} |
| 1 | 15-70 | 10,000 | 561.034 | 727.028 | 889.891 | 1116.340 | 1542.649 |
| 2 | 15-50 | 8000 | 530.392 | 679.656 | 819.701 | 997.779 | 1319.367 |
| | 50-70 | 12,000 | 609.448 | 752.767 | 871.825 | 1007.863 | 1246.036 |
| 3 | 15-35 | 8000 | 625.621 | 791.137 | 936.625 | 1110.149 | 1416.620 |
| | 35-50 | 10,000 | 610.134 | 752.405 | 871.710 | 1010.536 | 1244.382 |
| | 50-70 | 12,000 | 609.448 | 752.767 | 871.825 | 1007.863 | 1246.036 |
| 4 | 15-20 | 6000 | 587.206 | 725.078 | 839.469 | 971.4863 | 1194.847 |
| | 20-35 | 8000 | 605.615 | 755.268 | 882.203 | 1029.671 | 1283.605 |
| | 35-50 | 10,000 | 610.134 | 752.405 | 871.710 | 1010.536 | 1244.382 |
| | 50-70 | 12,000 | 609.448 | 752.767 | 871.825 | 1007.863 | 1246.036 |
| 5 | 15-20 | 6000 | 587.206 | 725.078 | 839.469 | 971.486 | 1194.847 |
| | 20-35 | 8000 | 605.615 | 755.268 | 882.203 | 1029.671 | 1283.605 |
| | 35-50 | 10,000 | 610.134 | 752.405 | 871.710 | 1010.536 | 1244.382 |
| | 50-65 | 12,000 | 625.897 | 772.407 | 892.313 | 1029.095 | 1266.288 |
| | 65-70 | 14,000 | 676.907 | 825.934 | 948.774 | 1096.816 | 1356.507 |

Abbreviations:
AUC0-12 = area under the plasma concentration-time curve from 0 to 12;
BID = twice daily
^{a}Value used in the exposure multiple calculation

The dose simulation modeling showed that a four-level model of weight dosing bands with fixed doses of 6000 mg BID, 8000 mg BID. 10,000 mg BID, or 12,000 mg BID (Table 3) would result in many subjects receiving exposures within the target range at weights from 12 to 100 kg.

TABLE 3
Designed Dosing Regimen for Trofinetide

| | | |
|---|---|---|
| Weight Band | Dose BID | Total Daily Dose |

| | | | |
|---|---|---|---|
| 12 to 20 kg | 6000 | mg | 12,000 mg |
| 21 to 35 kg | 8000 | mg | 16,000 mg |
| 36 to 50 kg | 10,000 | mg | 20,000 mg |
| 51 to 100 kg | 12,000 | mg | 24,000 mg |

### Example 2: Clinical Trial Protocol Using Herein Specified Dosing

Protocol Title a Randomized, Double-Blind, Placebo-Controlled, Parallel-Group Study of Trofinetide for the Treatment of Girls and Women with Rett Syndrome

Name of Investigational Product: Trofinetide Oral Solution

Rett syndrome is a devastating disorder for which there is as yet no treatment beyond symptomatic care resulting in a great unmet medical need. In the two studies of trofinetide in girls and women with RTT, trofinetide has been well tolerated. The highest dose assessed, 200 mg/kg BID, was observed to provide benefit compared to placebo even in a relatively small study, as described above. The rationale for the present study is to assess whether treatment with trofinetide does show a statistically significant benefit compared to placebo in a well-powered study.

Accordingly, the present study is designed to assess whether the effects of administration of trofinetide seen in the two Phase 2 studies, are confirmed in a larger population of children, adolescents and young adults.

This study will investigate effects in females only, and only in subjects who have typical RTT and a confirmed mutation of the MECP2 gene. The enrollees will be in a stable phase of their RTT; that is, they will not be undergoing active neurological regression. The exclusion of subjects with atypical RTT, subjects without a documented disease-causing MECP2 mutation, and male subjects is based on considerations of study design that will allow for a more homogeneous study population and, it is hoped, reduce variability that will allow observation of a difference between treatment with trofinetide and treatment placebo in this relatively small sample of the population.

The upper limit of the allowed age range has been increased to 20 years of age as compared to 15 years of age in the most recent Phase 2 study. The age range is not over 20 years in large part because in the United States school districts generally provide services until 20 or 21 years of age and the availability of services after that age is not consistent across the states.

The study will compare the efficacy and safety of a single trofinetide treatment group to treatment with placebo. Based on the results of the previous study treatment with trofinetide, 200 mg/kg BID was targeted. However, it was observed that subjects with lower body weights tended to have lower than expected exposure than did subjects who weighed more. Identification of body weight as a covariate has often been observed with drugs showing similar distributional properties, especially when considering adolescents or other groups with different demographic characteristics (Jusko et al. 1982; Kersting et al. 2012; Piana et al. 2014). A best practice approach to addressing this covariate is the development of a dosing model that accounts for the effect of weight on exposure. A model of simulated dose levels was developed as described in Example 1 to characterize the dose levels required to reach a minimal target exposure level of orally administered trofinetide at different weights in a pediatric population. The dose simulation modeling showed that a four-level model of weight dosing bands with fixed doses of 6 g BID, 8 g BID, 10 g BID or 12 g BID (Table 3, supra) would result in an optimal percentage of subjects with exposures within the target range at weights 12 kg-100 kg.

This study will be conducted as a multicenter, randomized, double-blind, placebo-controlled, parallel-group study in girls and women with Rett syndrome. The study will compare one active treatment group receiving weight-banded doses of trofinetide with a placebo group. Subjects will be stratified according to age stratum (5-10 years old, 11-15 years old, and 16-20 years old) and Baseline RSBQ severity (<35 total score and >35 total score). A minimum of 12 subjects are required to be randomized for each age stratum. The Sponsor, subjects, caregivers, and Investigators will be blinded to treatment assignment. The duration of participation for individual study subjects will be approximately 19 weeks. Approximately 18 sites will participate in this study.

The study will have 3 periods:
Screening period: up to 3 weeks
Double-blind Treatment period: 12 weeks
Safety follow-up period: 30 days

The study completion date is defined as the date the final subject, across all sites, completes their final protocol-defined assessment. An individual subject is considered to have completed the study on the date of her final protocol-defined assessment. Please note that the ‘final protocol-defined assessment’ includes the follow-up visit or contact, whichever is later.

Primary Objective
To investigate the efficacy of treatment with oral trofinetide versus placebo in girls and women with Rett syndrome

Co-Primary Endpoints
Rett Syndrome Behaviour Questionnaire (RSBQ) total score—Change from Baseline to Week 12
Clinical Global Impression-Improvement (CGI-I) Score at Week 12 Key Secondary Objective
To investigate the efficacy of treatment with oral trofinetide versus placebo on ability to communicate in girls and women with Rett syndrome

Key Secondary Endpoint

Change from Baseline to Week 12 in:
Communication and Symbolic Behavior Scales Developmental Profile™ Infant-Toddler Checklist-Social Composite Score (CSBS-DP-IT Social)

Other Secondary Objectives
To investigate the benefit of treatment with oral trofinetide versus placebo on overall quality of life for girls and women with Rett syndrome
To investigate the efficacy of treatment with oral trofinetide versus placebo in girls and women with Rett syndrome on:
   Hand function
   Ambulation and other gross motor skills
   Ability to communicate choices and preferences o Ability to communicate verbally
To investigate the efficacy of treatment with oral trofinetide versus placebo on a global assessment of the severity of illness in girls and women with Rett syndrome
To investigate the benefit of treatment with oral trofinetide versus placebo on the burden on caregivers of girls and women with Rett syndrome
To investigate the benefit of treatment with oral trofinetide versus placebo on the impact of the disability on the child's and family's everyday life

Other Secondary Endpoints

Change from Baseline to Week 12 in:
Overall Quality of Life Rating of the Impact of Childhood Neurologic Disability Scale (ICND)
Rett Syndrome Clinician Rating of Hand Function (RTT-HF) Rett Syndrome Clinician Rating of Ambulation and Gross Motor Skills (RTT-AMB)
Rett Syndrome Clinician Rating of Ability to Communicate Choices (RTT-COMC)
Rett Syndrome Clinician Rating of Verbal Communication (RTT-VCOM)
Clinical Global Impression-Severity (CGI-S)
Rett Syndrome Caregiver Burden Inventory (RTT-CBI) Total Score (items 1-24)
Impact of Childhood Neurologic Disability Scale (ICND) Total Score

Safety Objective
To investigate the safety and tolerability of treatment with oral trofinetide versus placebo in girls and women with Rett syndrome

Safety Endpoints
Treatment-emergent adverse events (TEAEs)
Serious adverse events (SAEs)
Withdrawals due to adverse events
Potentially clinically important changes in other safety assessments

Pharmacokinetic Objectives
To characterize the pharmacokinetics (PK) of trofinetide in girls and women with Rett syndrome
To assess the pharmacokinetic/pharmacodynamic (PK/PD) relationship using safety and efficacy endpoints in girls and women with Rett syndrome

Pharmacokinetic Endpoints
Whole blood concentration of trofinetide and possible metabolites
Trofinetide PK parameters using the population PK approach⋅PK/PD using appropriate PK/PD analysis methods

Number of Study Sites

Approximately 18 sites will participate in this study.

Number of Subjects Planned

184 subjects are expected to be randomized (with a minimum of 12 subjects randomized for three age ranges [5-10 years old, 11-15 years old, and 16-20 years old]) with a total of 92 subjects per treatment arm.

Test Product, Dose, and Administration

Subjects will receive an oral dose of trofinetide or placebo, for up to 12 weeks. Dose will be based on the subject's weight at Baseline, as outlined below. Doses may be administered by gastrostomy (G) tube (doses administered via gastrojejunal [GJ] tubes must be administered through the G-port).

TABLE 4
Dosing Schedule Based on Weight at Baseline

| | | |
|---|---|---|
| Weight | Dose | Total Daily Dose |

| | | | | | |
|---|---|---|---|---|---|
| 12-20 | kg | 30 mL | (6 g) | BID 60 mL | (12 g) |
| >20-35 | kg | 40 mL | (8 g) | BID 80 mL | (16 g) |
| >35-50 | kg | 50 mL | (10 g) | BID 100 mL | (20 g) |
| >50 | kg | 60 mL | (12 g) | BID 120 mL | (24 g) |

Abbreviations: BID = twice daily

Study Design

This is a 12-week, multicenter, randomized, double-blind, placebo-controlled, parallel group study. The study will compare one active treatment group receiving weight-banded doses of trofinetide with a placebo group. Subjects will be stratified according to age stratum (5-10 years old, 11-15 years old, and 16-20 years old) and Baseline RSBQ severity (<35 total score and >35 total score). The Sponsor, subjects, caregivers, and Investigators will be blinded to treatment assignment.

The study will have 3 periods:
Screening period: up to 3 weeks
Double-blind Treatment period: 12 weeks
Safety follow-up period: 30 days

Screening Period (Up to 3 Weeks)

During the Screening period, subjects will be assessed for study eligibility. Only those subjects who meet all inclusion and no exclusion criteria will be eligible for the study. Investigators should not withdraw a subject's prohibited medication for the purpose of enrolling them into the study. Medications should be discontinued only if it is deemed clinically appropriate to do so and in consultation with the treating physician. Subjects will be evaluated for the diagnosis of Rett syndrome. In addition, there must be verified documentation of a MECP2 mutation. Genotyping may be done as part of the study if documentation is not adequate. Caregivers will begin to keep a semi-structured caregiver diary during the screening period.

Double-Blind Treatment Period (12 Weeks)

The Baseline visit (Visit 2) may occur after screening procedures are completed and have not ruled the subject out of eligibility for the study. At Visit 2, and upon confirmation of eligibility, subjects will be randomized in a 1:1 ratio to trofinetide oral solution or matching placebo. Dose will be based on weight as outlined in Table 4. The first dose of study drug will be administered at the study site after all Baseline assessments are completed, or, if the Investigator judges that it is too late in the day, on the following day. The day the first dose is taken will be considered Day 1 of dosing. An ECG must be performed 2-3 hours after the first dose and a PK sample will be taken upon completion of the ECG. Study drug must be discontinued in the event that a post-randomization QTcF duration of ≥500 ms or an increase of ≥60 ms compared to the average QTcF interval at Baseline (before dosing) is observed.

Dosing is twice a day, once in the morning and once in the evening. The subject should not eat for 1 hour before dose administration and for 1 hour after dose administration. In addition to the study drug dispensed at the site at the Baseline visit, additional investigational product will be shipped directly to the subject or visiting nurse. Study drug shipment, return, and accountability will be performed in accordance with the drug distribution plan. Each site will also have a plan for distribution of drug to the subjects. Confirmation of any delivery to the subject's home will be made by a visiting nurse at a home visit. Subjects will return to the clinic for assessments at Week 2, Week 6, and Week 12/early termination (ET).

Safety Follow-Up Period (30 Days)

Subjects who do not continue into the open-label extension study will receive a follow-up telephone call to assess safety 30 days after the last dose of study drug.

Study Duration

The duration of participation for individual study subjects will be approximately 19 weeks, consisting of a screening period of up to 3 weeks, a treatment period of 12 weeks, and a safety follow-up period of 30 days. The study completion date is defined as the date the final subject, across all sites, completes their final protocol-defined assessment.

Main Criteria for Inclusion and Exclusion

To be eligible for this study, subjects must meet all of the inclusion criteria and none of the exclusion criteria.

Inclusion Criteria:
1. Informed consent prior to the conduct of any study procedures is required as follows:
   a. For subjects who are minors: written informed consent will be obtained from the legally acceptable representative (LAR). The subject should provide written or oral assent if deemed able by the Investigator. The process of obtaining informed consent will be conducted in accordance with institutional review board (IRB) or ethics committee (EC) policy and applicable local law.
   b. For subjects who are not minors: written informed consent will be obtained from the LAR or the subject if deemed able by the Investigator. If the subject is deemed not able to provide consent, the subject should provide written or oral assent if deemed able by the Investigator. The process of obtaining informed consent will be conducted in accordance with IRB or EC policy and applicable local law.
   c. The subject's caregiver must also provide informed consent regarding their participation in the study prior to participating in any study procedures.
2. Female subjects 5 to 20 years of age, inclusive, at Screening
3. Body weight ≥12 kg at Screening
4. Can swallow the study medication provided as a liquid solution or can take it by gastrostomy tube
5. The subject's caregiver has sufficient language skills to complete the caregiver assessments in the language in which the study assessments are provided

Diagnosis
6. Has classic/typical Rett syndrome (RTT)
7. Has a documented disease-causing mutation in the MECP2 gene
8. Is post-regression at Screening, defined as: No loss or degradation of ambulation (including gait, coordination, independence of walking/standing) within 6 months of Screening
   b. No loss or degradation of hand function within 6 months of Screening
   c. No loss or degradation of speech (including babbling, words or previously developed communicative vocalizations) within 6 months of Screening
   d. No loss or degradation of non-verbal communicative or social skills (including eye gaze, using body to indicate communicative intent, social attentiveness) within 6 months of Screening
9. Has a severity rating of 10-36, inclusive, on the Rett Syndrome Clinical Severity Scale at Screening
10. Has a CGI-S score of ≥4 at Screening and Baseline

Concomitant Treatment
11. If the subject is taking or was taking an anticonvulsant or any other psychoactive medication (including cannabinoids):
   a. the treatment regimen has been stable for at least 4 weeks before Baseline and there is no current plan to change the dose, OR
   b. if the medication was discontinued, the discontinuation has occurred no fewer than 2 weeks or 5 half-lives (whichever is greater) before Baseline
12. If the subject is taking or was taking any other medication daily for a chronic illness (not including antibiotics, pain relievers, and laxatives):
   a. the treatment regimen of the medication has been stable for at least 4 weeks before Baseline and there is no current plan to change the dose, OR
   b. if the medication was discontinued, the discontinuation has occurred no fewer than 2 weeks or 5 half-lives (whichever is greater) before Baseline 13. If the subject is receiving or was receiving a non-pharmacologic somatic treatment (e.g., a ketogenic diet or vagal nerve stimulation):
      a. the treatment regimen has been stable for at least 4 weeks before Baseline and there is no current plan to change the treatment, OR
      b. if the treatment was discontinued, the discontinuation has occurred no fewer than 2 weeks before Baseline
14. If the subject is receiving or was receiving non-pharmacologic treatments such as an educational, behavioral, physical, occupational, or speech therapy:
   a. the treatment regimen has been stable for at least 4 weeks before Baseline and there is no current plan to change the treatment (Note: changes to a treatment regimen that are due to school schedules or are otherwise seasonally related are not exclusionary), OR
   b. if the treatment was discontinued, the discontinuation has occurred no fewer than 2 weeks before Baseline

Seizures
15. Has a stable pattern of seizures, or has had no seizures, within 8 weeks of Screening

Childbearing Potential
16. Subjects of childbearing potential must abstain from sexual activity for the duration of the study and for at least 30 days thereafter. If a subject is sexually active or becomes sexually active during the study, she must use 2 clinically acceptable methods of contraception (e.g., oral, intrauterine device [IUD], diaphragm plus spermicide, injectable, transdermal or implantable contraception) for the duration of the study and for at least 30 days thereafter. Subject must not be pregnant or breastfeeding.

Exclusion Criteria:

Concomitant Treatment
1. Has been treated with growth hormone within 12 weeks of Baseline
2. Has been treated with IGF-1 within 12 weeks of Baseline
3. Has been treated with insulin within 12 weeks of Baseline

Medical Conditions Other than Rett Syndrome
4. Has current clinically significant cardiovascular, endocrine (such as hypo- or hyperthyroidism, Type 1 diabetes mellitus, or uncontrolled Type 2 diabetes mellitus), renal, hepatic, respiratory or gastrointestinal disease (such as celiac disease or inflammatory bowel disease) or has major surgery planned during the study
5. Has a history of, or current, cerebrovascular disease or brain trauma
6. Has significant, uncorrected visual or uncorrected hearing impairment
7. Has a history of, or current, malignancy

Laboratory Studies, Vital Signs, and Electrocardiogram
8. Has a clinically significant abnormal laboratory value at Screening. Laboratory testing may be repeated during the Screening period with agreement of the Medical Monitor.
9. Has serum potassium below the normal range for the subject (according to the central laboratory) at Screening. Serum potassium may be repeated during the Screening period with the agreement of the Medical Monitor.
10. Has a hemoglobin A1C (HbA1c) >7.0% at Screening
11. Has a thyroid stimulating hormone (TSH) value outside the normal range for the subject (according to the central laboratory) at Screening
12. Has clinically significant abnormality in vital signs at Screening or Baseline
13. Has any of the following:
   a. QTcF interval of >450 ms at Screening or Baseline (before dosing)
   b. History of a risk factor for torsades de pointes (e.g., heart failure or family history of long QT syndrome)
   c. History of clinically significant QT prolongation that is deemed to put the subject at increased risk of clinically significant QT prolongation
14. Has any other clinically significant finding on ECG at Screening or Baseline (before dosing)
15. Has a positive pregnancy test at Screening

Other Criteria
16. Has a significant sensitivity or allergic reaction to trofinetide or its excipients
17. Has participated in another interventional clinical study within 30 days prior to Screening
18. Is judged by the Investigator or the Medical Monitor to be inappropriate for the study for any reason

Pharmacokinetic Assessments

PK blood samples will be collected at 5 timepoints for trofinetide concentration measurements at the Baseline visit (both before dosing and approximately 2-3 hours after dosing) and at Visit 3, Visit 4, and Visit 5/early termination (ET). The second PK sample taken at the Baseline visit (Visit 2) approximately 2-3 hours after dosing should take place as soon as possible after the postdose ECG is performed. PK samples at Visits 3, 4, and 5 should be collected at one of the following time intervals:
2-3 hours after dosing
3-7 hours after dosing
7-11 hours after dosing

Over the duration of the study, every effort should be made to collect the PK samples across Visits 3, 4, and 5 at each one of the different time intervals (2-3 hours after dosing, 3-7 hours after dosing, and 7-11 hours after dosing). If samples are taken within the same time interval across visits, every effort should be made to collect the samples at different times within the specified time interval. Pharmacokinetic samples will also be collected, if possible, at any ET visit or the visit immediately following any SAE or following any AE leading to discontinuation.

For all PK samples (scheduled and unscheduled), the dates and times of administration of the last 3 doses of study drug, the dates and times of the meals closest to the times those doses were given, as well as the date and time of the sample draw should be recorded. For samples collected from subjects who experience any SAE or experience an AE leading to discontinuation, the date and time of the last dose of study drug prior to the SAE or AE should also be recorded.

Optional Biomarker Analysis

Participation in the effort to identify biomarkers is an optional component of the study. Subjects for whom separate informed consent for the identification of biomarkers of response to trofinetide is provided will have blood samples taken at Baseline (before dosing) and at Visit 5/ET. Blood samples will be used to investigate differences between responders and non-responders in both trofinetide-treated and placebo-treated subjects in RNA transcripts (transcriptomics), proteins (proteomics), and metabolites (metabolomics).

Sample Size Calculations

The sample size calculation was performed for the co-primary endpoints as a family of two hypothesis tests at an overall two-sided significance level of 0.05. A total sample size of 174 evaluable subjects in a 1:1 ratio to trofinetide or placebo was estimated to provide at least 90% power for the hypothesis testing family assuming the following treatment differences (SD) estimated from Phase 2 study data: −4.4 (8) for the mean change from Baseline to Week 12 in the RSBQ total score and −0.5 (0.7) for the CGI-I mean score at Week 12.

The sample size of 174 evaluable subjects will provide at least 95% power at a two-sided significance level of 0.05 for each individual hypothesis test within the family. Trofinetide will be superior to placebo if both hypothesis tests within the family are shown to be statistically significant at 0.05. Based on data from Phase 2 study, the correlation between the RSBQ total score and the CGI-I score is low, so the measures are assumed to be independent. Therefore, the overall power to detect a treatment difference on both of the co-primary endpoints will be at least 90% (0.952). Adjusting for an anticipated discontinuation rate of up to 5%, approximately 184 subjects will be randomized in a 1:1 ratio to trofinetide or placebo.

Statistical Methods

Analysis Sets

The following populations will be defined and used in the analysis:

Safety Analysis Set

The Safety Analysis Set will consist of all randomized subjects who received at least one dose of study medication. The Safety Analysis Set will be analyzed according to the actual treatment received.

Full Analysis Set (FAS)

The FAS will consist of all randomized subjects who received at least one dose of study medication and who have both a Baseline value and at least one post-Baseline value for the RSBQ total score or who have at least one post-Baseline value for the CGI-I score. The FAS will be analyzed according to the treatment they were assigned regardless of the actual treatment received.

Per-Protocol (PP) Analysis Set

The PP Analysis Set will consist of the subjects in FAS who did not have a major protocol violation that would affect interpretation of the efficacy data. The PP Analysis Set will be defined prior to study unblinding. The PP Analysis Set will be analyzed according to the actual treatment received.

Pharmacokinetic (PK) Analysis Set

The PK Analysis Set will consist of subjects in the Safety Analysis Set with at least one measurable trofinetide whole blood concentration.

Descriptive Statistics

Unless stated otherwise, all statistical tests will be 2-sided using a 5% significance level, leading to 95% (2-sided) confidence intervals. Trofinetide will be superior to placebo if both co-primary endpoints are shown to be statistically significant. Continuous measurement results will be reported using the number of subjects with data values, mean, standard error of the mean, median, standard deviation, minimum, and maximum. For each categorical outcome, the number and percentage of subjects in each category will be reported.

A hierarchical approach will be used to control for the multiple endpoints (co-primary and secondary).

Primary Analysis

The co-primary efficacy endpoints will be analyzed using a mixed model for repeated measures (MMRM). An unstructured covariance matrix will be used and the Kenward-Roger approximation will be used to adjust the denominator degrees of freedom. The treatment comparisons will be based on the difference in least squares means at Week 12.

For the change from Baseline in the RSBQ total score, the MMRM model will include effects for treatment group, age group (5-10 years old, 11-15 years old, and 16-20 years old), visit, Baseline RSBQ total score, and interactions for treatment group by visit and Baseline RSBQ Total Score by Visit.

For the CGI-I score, the MMRM model will include effects for treatment group, age group (5-10 years old, 11-15 years old, and 16-20 years old), visit, Baseline RSBQ severity (<35 total score and ≥35 total score), Baseline CGI-S score, and interactions for treatment group by visit and Baseline CGI-S score by visit.

Sensitivity analyses will be performed to assess the impact of missing data, including analyses based on a missing not at random assumption.

Secondary Analyses

The key secondary endpoint will be analyzed using a MMRM method with effects for treatment group, age group (5-10 years old, 11-15 years old, and 16-20 years old), visit, Baseline RSBQ severity (<35 total score and >35 total score), Baseline CSBS-DP-IT Social score, and interactions for treatment group by visit and Baseline CSBS-DP-IT Social score by visit. An unstructured covariance matrix will be used and the Kenward-Roger approximation will be used to adjust the denominator degrees of freedom. The treatment comparisons will be based on the difference in least squares means at Week 12.

For the other secondary endpoints that are assessed at multiple post-Baseline visits, the change from Baseline will be analyzed using a MMRM analysis similar to those described above for the co-primary endpoints. The MMRM model will include effects for treatment group, age group (5-10 years old, 11-15 years old, and 16-20 years old), visit, Baseline RSBQ severity (<35 total score and ≥35 total score), Baseline score, and interactions for treatment group by visit and Baseline score by visit.

For the other secondary endpoints that are assessed at a single post-Baseline visit (i.e. Week 12 only), the change from Baseline will be analyzed using an analysis of covariance (ANCOVA) model with effects for treatment group, age group (5-10 years old, 11-15 years old, and 16-20 years old), Baseline RSBQ severity (<35 total score and ≥35 total score), and Baseline score.

Safety Analyses

Safety results will be summarized by treatment group using descriptive statistics. No formal statistical testing will be performed for any of the safety endpoints. Adverse events will be classified into standard terminology using the Medical Dictionary for Regulatory Activities (MedDRA). Treatment-emergent adverse events (TEAEs), TEAEs leading to discontinuation, TEAEs related to study drug, TEAEs by maximum severity, fatal TEAEs, serious adverse events (SAEs), and SAEs related to study drug will all be summarized. Descriptive statistics for ECG, vital signs and weight, and clinical laboratory parameters, including changes from Baseline, will be tabulated by timepoint. Additionally, categorical analyses will be conducted on the incidence of subjects with prolonged QTc intervals and changes in QTc intervals in accordance with International Council on Harmonisation (ICH) guidelines.

Pharmacokinetic Analyses

Pharmacokinetic (PK) and efficacy (PD) measures will be collected from all subjects at the Baseline (Week 0) visit before dosing, at the Baseline (Week 0) visit after dosing, and after dosing at Weeks 2, 6, and 12/EOT.

Whole blood concentration and possible metabolites data for trofinetide will be listed and summarized using descriptive statistics. If data allow, population PK and PK/PD analyses will be performed to further characterize the PK profile and exposure response relationship of trofinetide using measures of safety and efficacy parameters. Trofinetide whole blood concentration data will remain blinded until the unblinding of the clinical database at the end of the study.

Screening Period (Up to 3 Weeks)

During the Screening period, subjects will be assessed for study eligibility. Only those subjects who meet all inclusion and no exclusion criteria will be eligible for the study. Investigators should not withdraw a subject's prohibited medication for the purpose of enrolling them into the study. Medications should be discontinued only if it is deemed clinically appropriate to do so and in consultation with the treating physician. Subjects will be evaluated for the diagnosis of Rett syndrome. In addition, there must be verified documentation of a MECP2 mutation. Genotyping may be done as part of the study if documentation is not adequate.

Caregivers will begin to keep a semi-structured caregiver diary during the screening period.

Double-Blind Treatment Period (12 Weeks)

The Baseline visit (Visit 2) may occur after screening procedures are completed and have not ruled the subject out of eligibility for the study. At Visit 2, and upon confirmation of eligibility, subjects will be randomized in a 1:1 ratio to trofinetide oral solution or matching placebo. Dose will be based on the subject's weight at Baseline as outlined in Table 3, supra. Doses may be administered by gastrostomy (G) tube (doses administered via gastrojejunal [GJ] tubes must be administered through the G-port).

The first dose of study drug will be administered at the study site after all Baseline assessments are completed, or, if the Investigator judges that it is too late in the day, on the following day. The day the first dose is taken will be considered Day 1 of dosing. An ECG must be performed 2-3 hours after first dose and a PK sample will be taken upon completion of the ECG.

Dosing is twice a day, once in the morning and once in the evening. The subject should not eat for 1 hour before dose administration and for 1 hour after dose administration.

In addition to the study drug dispensed at the site at the Baseline visit, additional investigational product will be shipped directly to the subject or visiting nurse. Study drug shipment, return, and accountability will be performed in accordance with the drug distribution plan. Each site will also have a plan for distribution of drug to the subjects. Confirmation of any delivery to the subject's home will be made by a visiting nurse at a home visit.

Subjects will return to the clinic for assessments at Week 2, Week 6, and Week 12/early termination (ET). For the 2 days before clinic visits and the morning of the clinic visit, the dates and times of trofinetide dosing, concomitant medication dosing, and meals should be recorded in the caregiver diary.

Safety Follow-Up Period (30 Days)

A 30 day safety follow-up telephone contact is to be completed for subjects who complete the treatment period of the study and decide not to continue into the open-label study or are not eligible for the open-label study, as well as those who discontinue prematurely from the study. The telephone contact includes assessment of concomitant medications and treatments and assessment of AEs.

### Subject Eligibility and Withdrawal Criteria

To be eligible for this study, subjects must meet all of the inclusion criteria and none of the exclusion criteria.

Inclusion Criteria

A subject must meet all of the following inclusion criteria to be eligible for participation in the study:
1. Informed consent prior to the conduct of any study procedures is required as follows:
   a. For subjects who are minors: written informed consent will be obtained from the legally acceptable representative (LAR). The subject should provide written or oral assent if deemed able by the Investigator. The process of obtaining informed consent will be conducted in accordance with institutional review board (IRB) or ethics committee (EC) policy and applicable local law.
   b. For subjects who are not minors: written informed consent will be obtained from the LAR or the subject if deemed able by the Investigator. If the subject is deemed not able to provide consent, the subject should provide written or oral assent if deemed able by the Investigator. The process of obtaining informed consent will be conducted in accordance with IRB or EC policy and applicable local law.
   c. The subject's caregiver must also provide informed consent regarding their participation in the study prior to participating in any study procedures.
2. Female subjects 5 to 20 years of age, inclusive, at Screening
3. Body weight ≥12 kg at Screening
4. Can swallow the study medication provided as a liquid solution or can take it by gastrostomy tube
5. The subject's caregiver has sufficient language skills to complete the caregiver assessments in the language in which the study assessments are provided

Diagnosis
6. Has classic/typical Rett syndrome (RTT)
7. Has a documented disease-causing mutation in the MECP2 gene
8. Is post-regression at Screening, defined as:
   a. No loss or degradation of ambulation (including gait, coordination, independence of walking/standing) within 6 months of Screening
   b. No loss or degradation of hand function within 6 months of Screening
   c. No loss or degradation of speech (including babbling, words or previously developed communicative vocalizations) within 6 months of Screening
   d. No loss or degradation of nonverbal communicative or social skills (including eye gaze, using body to indicate communicative intent, social attentiveness) within 6 months of Screening
9. Has a severity rating of 10-36, inclusive, on the Rett Syndrome Clinical Severity Scale at Screening
10. Has a CGI-S score of ≥4 at Screening and Baseline

Concomitant Treatment
11. If the subject is taking or was taking an anticonvulsant or any other psychoactive medication (including cannabinoids):
   a. the treatment regimen has been stable for at least 4 weeks before Baseline and there is no current plan to change the dose, OR
   b. if the medication was discontinued, the discontinuation has occurred no fewer than 2 weeks or 5 half-lives (whichever is greater) before Baseline
12. If the subject is taking or was taking any other medication daily for chronic illness (not including antibiotics, pain relievers, and laxatives):
   a. the treatment regimen of the medication has been stable for at least 4 weeks before Baseline and there is no current plan to change the dose, OR
   b. if the medication was discontinued, the discontinuation has occurred no fewer than 2 weeks or 5 half-lives (whichever is greater) before Baseline
13. If the subject is receiving or was receiving a non-pharmacologic somatic treatment (e.g., a ketogenic diet or vagal nerve stimulation):
   a. the treatment regimen has been stable for at least 4 weeks before Baseline and there is no current plan to change the treatment, OR
   b. if the treatment was discontinued, the discontinuation has occurred no fewer than 2 weeks before Baseline
14. If the subject is receiving or was receiving non-pharmacologic treatments such as an educational, behavioral, physical, occupational, or speech therapy:
   a. the treatment regimen has been stable for at least 4 weeks before Baseline and there is no current plan to change the treatment (Note: changes to a treatment regimen that are due to school schedules or are otherwise seasonally related are not exclusionary), OR
   b. if the treatment was discontinued, the discontinuation has occurred no fewer than 2 weeks before Baseline

Seizures
15. Has a stable pattern of seizures, or has had no seizures, within 8 weeks of Screening

Childbearing Potential
16. Subjects of childbearing potential must abstain from sexual activity for the duration of the study and for at least 30 days thereafter. If a subject is sexually active or becomes sexually active during the study, she must use 2 clinically acceptable methods of contraception (e.g., oral, intrauterine device [IUD], diaphragm plus spermicide, injectable, transdermal or implantable contraception) for the duration of the study and for at least 30 days thereafter. Subject must not be pregnant or breastfeeding.

Exclusion Criteria

A subject must meet none of the following exclusion criteria to be eligible for the study:

Concomitant Treatment
1. Has been treated with growth hormone within 12 weeks of Baseline
2. Has been treated with IGF-1 within 12 weeks of Baseline
3. Has been treated with insulin within 12 weeks of Baseline

Medical Conditions Other than Rett Syndrome
4. Has current clinically significant cardiovascular, endocrine (such as hypo- or hyperthyroidism, Type 1 diabetes mellitus, or uncontrolled Type 2 diabetes mellitus), renal, hepatic, respiratory or gastrointestinal disease (such as celiac disease or inflammatory bowel disease) or has major surgery planned during the study
5. Has a history of, or current, cerebrovascular disease or brain trauma
6. Has significant, uncorrected visual or uncorrected hearing impairment
7. Has a history of, or current, malignancy

Laboratory Studies, Vital Signs, and Electrocardiogram
8. Has a clinically significant abnormal laboratory value at Screening. Laboratory testing may be repeated during the Screening period with agreement of the Medical Monitor.
9. Has serum potassium below the normal range for the subject (according to the central laboratory) at Screening. Serum potassium may be repeated during the Screening period with the agreement of the Medical Monitor.
10. Has a hemoglobin A1C (HbA1c) >7% at Screening
11. Has a thyroid stimulating hormone (TSH) value outside the normal range for the subject (according to the central laboratory) at Screening
12. Has clinically significant abnormality in vital signs at Screening or Baseline
13. Has any of the following:
   a. QTcF interval of >450 ms at Screening or Baseline (before dosing)
   b. History of a risk factor for torsades de pointes (e.g., heart failure or family history of long QT syndrome)
   c. History of clinically significant QT prolongation that is deemed to put the subject at increased risk of clinically significant QT prolongation
14. Has any other clinically significant finding on ECG at Screening or Baseline (before dosing)
15. Has a positive pregnancy test at Screening

Other Criteria
16. Has a significant sensitivity or allergic reaction to trofinetide or its excipients
17. Has participated in another interventional clinical study within 30 days prior to Screening
18. Is judged by the Investigator or the Medical Monitor to be inappropriate for the study for any reason

Subject Withdrawal of Consent

In accordance with the Declaration of Helsinki and other applicable regulations, a subject and legally acceptable representatives consenting on behalf of subjects have the right to withdraw from the study at any time, and for any reason, without prejudice to his or her future medical care.

Should a subject (or LAR) request or decide to withdraw consent, every reasonable effort should be made to complete and report observations as thoroughly as possible up to the date of withdrawal, including the evaluations specified at the ET or safety follow-up (whichever is applicable).

Subject or Study Discontinuation

Subjects may be discontinued from the study for a number of reasons, including, but not limited to, those listed below:
Adverse event
Death
Increase in post-Baseline QTcF interval (defined below)
Lack of efficacy
Lost to follow-up
Non-compliance with study drug
Physician decision
Pregnancy
Protocol deviation
Study terminated by sponsor
Use of prohibited medication
Other

The Sponsor reserves the right to discontinue the study at any time for any reason. Such reasons may be any of, but not limited to, the following:
Occurrence of AEs unknown to date in respect of their nature, severity, and duration or the unexpected incidence of known AEs
Medical, ethical or business reasons affecting the continued performance of the study
Regulatory authorities also have the right to terminate the conduct of the study in their region for any reason.

Post-Baseline QTcF Interval Stopping Criteria

Study drug must be discontinued in the event that a post-randomization QTcF duration of ≥500 ms or an increase of ≥60 ms compared to the average QTcF interval at Baseline (before dosing) is observed.

Handling of Subject Discontinuation During the Treatment Period

Unless the subject has withdrawn consent (or the LAR has withdrawn consent on behalf of the subject) to be contacted for this study, every reasonable effort should be made to complete Visit 5/early termination (ET) and the safety follow-up (as outlined in Table S-2) if a subject discontinues prematurely for any reason. All information will be reported on the applicable pages of the electronic case report form (eCRF).

If a subject is discontinued from the study because of an AE, every reasonable attempt should be made to follow the subject until the AE resolves or until the Investigator deems the AE to be chronic or stable. For subjects who continue to be followed for safety, SAEs should continue to be reported as described in Section 7.3.2. All SAEs will continue to be followed until such events have resolved or the Investigator deems them to be chronic or stable.

Pharmacokinetic samples will also be collected, if possible, at any ET visit or the visit immediately following any SAE or following any AE leading to discontinuation, even if it is an unscheduled visit.

Subject Lost to Follow-Up

A subject will be considered lost to follow-up if they fail to attend a scheduled visit (excluding the safety follow-up telephone call) and the study site is unable to contact the subject or caregiver.

Every reasonable effort should be made to contact the caregiver and will include a minimum of 3 documented telephone calls (each performed at different times of the day) and, if necessary, a certified letter to the caregiver's last known mailing address or local equivalent methods. All contact attempts are to be documented in the source documents.

Prior and Concomitant Therapy

All medications used up to 8 weeks prior to Baseline through completion of the safety follow-up visit or ET are to be recorded.

In order to ensure that appropriate concomitant therapy is administered, it is essential that caregivers be instructed not to administer any medication to the subject without prior consultation with the Investigator (unless the subject is receiving treatment for a medical emergency).

The Investigator may prescribe appropriate medication to treat AEs. The Sponsor and Investigator or designee will confer to determine whether it is appropriate to continue such a subject in the trial if a prohibited medication is prescribed.

Every effort should be made to maintain stable regimens of concomitant medications and allowed non-medicine based therapies throughout the course of the study, with the understanding that there will be some changes to a treatment regimen that are due to school schedules or are otherwise seasonally related. Special cases are medications that have diarrhea as an acute side effect which should be monitored and may be adjusted as needed if diarrhea occurs. Concomitant treatments for chronic constipation should also be monitored.

Permitted and Prohibited Medications

Prohibited medications are IGF-1, growth hormone, and insulin. Prohibitions for concomitant medications should be followed between Visit 2 and Visit 5/ET. Medications that can prolong QT interval are not prohibited but should be used with caution. Any use of medications that could interfere with study conduct should be discussed with the Medical Monitor.

Use of medications that could interfere with study conduct or any questions regarding concomitant medications should be reviewed and/or discussed with the Medical Monitor.

Psychoactive concomitant medications should remain at a stable dose throughout the study if possible. Any non-pharmacologic somatic treatment regimen (e.g., a ketogenic diet or vagal nerve stimulation) that has CNS effects should remain stable throughout the study if possible. Treatment of constipation may be changed as needed.

Subjects who require current treatment with a prohibited medication will be withdrawn from the study.

Subjects who have previously taken a prohibited medication during the study will be withdrawn from the study unless:
the prohibited medication has been discontinued AND
withdrawal from the study presents an unacceptable medical risk to the subject

The justification to allow the subject to continue in the trial will be made by the Sponsor/Medical Monitor, with medical input from the Investigator, and will be documented. If a subject is allowed to remain in the trial, this will be reported as a major protocol deviation and not a waiver.

### Investigational Product

Investigational Product Description

The investigational product will be trofinetide oral solution or matching placebo for trofinetide oral solution. Dose will be based on weight as outlined in Table 4. Trofinetide will be provided in a ready-to-use aqueous solution for oral administration. Doses will be administered orally or by G-tube (doses administered by GJ tubes must be administered through the G-port).

Formulation, Appearance, and Packaging

The Sponsor will supply trofinetide oral solution and matching placebo for trofinetide oral solution as an aqueous, ready-to-use, strawberry-flavored liquid in 500 mL high density polyethylene (HDPE) plastic bottles with a child-resistant closure.

Trofinetide oral solution is a clear, red-colored liquid containing 1 gram of trofinetide in each 5 mL. The trofinetide oral solution also contains purified water, maltitol, strawberry flavor, sucralose, methylparaben sodium, propylparaben sodium, and FD&C Red #40 as inactive ingredients.

The placebo aqueous solution does not contain trofinetide active pharmaceutical ingredient (API), but contains purified water, acetic acid, caramel color, citric acid, lemon flavor, maltitol, methylparaben sodium, natural quinine flavor, propylparaben sodium, FD&C Red #40, strawberry flavor, sucralose, xanthan gum, and D&C Yellow #10.

Trofinetide and matched placebo are manufactured under current Good Manufacturing Practices.

During the treatment period, study drug will be distributed in a quantity sufficient to ensure the subject has an adequate supply of study drug between study visits.

Product Storage and Stability

Investigational product will be shipped refrigerated at a temperature between 2° C. and 8° C. (36° F. and 46° F.) and should be stored at this temperature. Do not freeze.

Dosing and Administration

The first dose of study drug will be administered at the study site after all Baseline assessments are completed, or, if the Investigator judges that it is too late in the day, on the following day. The day the first dose is taken will be considered Day 1 of dosing. An ECG must be performed 2-3 hours after the first dose and a PK sample will be taken upon completion of the ECG.

The dose is based on the subject's weight at Baseline (see Table 41). The dose will remain the same for each subject throughout the study even if the subject's weight changes. In addition to the study drug dispensed at the site at the Baseline visit, additional investigational product will be shipped directly to the subject or visiting nurse. Study drug shipment, return, and accountability will be performed in accordance with the drug distribution plan. Each site will also have a plan for distribution of drug to the subjects. Confirmation of any delivery to the subject's home will be made by a visiting nurse at a home visit.

Doses may be taken orally or via gastrostomy tube. For gastrojejunal tubes, medication should be given via the gastric port. The subject should not eat for 1 hour before dose administration and for 1 hour after dose administration. Subjects receiving continuous tube feeding may have their feeding administration stopped for study drug administration 1 hour before dose administration and for 1 hour after dose administration, if they can tolerate it.

Doses may be taken over a 10 minute period with a follow up of 250 mL of water. Dosing is twice a day, once in morning and once in afternoon or evening. There should be at least 8 hours between doses.

Method of Assigning Subjects to Treatment Groups

At Visit 2, eligible subjects who meet inclusion and do not meet exclusion criteria will be randomized in a 1:1 ratio to receive either trofinetide or placebo.

Blinding

Treatment assignments will be blinded to all study subjects, caregivers, Investigators, raters, site personnel, and Sponsor personnel. In the event of a potential suspected unexpected serious adverse reaction (SUSAR), in accordance with current health authority guidance, treatment assignments for the affected subject may be unblinded to a controlled group of the Sponsor's Safety and/or Regulatory personnel for reporting purposes. Details regarding medical emergency unblinding procedures are provided in Section 9.9.

Study Drug Compliance

If a subject misses one dose of study drug, she should not take an extra dose the next day.

Overdose

An overdose is a deliberate or inadvertent administration of a treatment at a dose higher than the maximum recommended dose per protocol. It must be reported, irrespective of outcome, even if toxic effects were not observed (Section 7.3.4). All events of overdose are to be captured as protocol deviations.

### Study Procedures

Study-specific procedures are detailed below.

Screening Assessments

Confirm Diagnosis of Rett Syndrome and MECP2 Mutation

The site will confirm that the subject meets criteria for typical/classic Rett syndrome and that there is documentation of disease-causing mutation of the MECP2 gene. The genotyping must have been performed at a laboratory certified by the College of American Pathologists (CAP), or under the Clinical Laboratory Improvement Act/Amendment (CLIA), or by an equivalent organization. If documentation of the mutation is not adequate, genomic testing for a mutation in the MECP2 gene may be conducted as part of Screening.

The documented mutation should be associated with Rett syndrome according to the MECP2 mutation database RettBASE (mecp2.chw.edu.au/cgibin/mecp2/search/search.cgi?form=combined) (Krishnaraj et al. 2017). If there is any question regarding the association of the mutation with Rett syndrome, the Medical Monitor must be consulted. The disease-causing mutation will be documented in the source documentation and eCRF.

Medical History, Including Rett Syndrome History, and Demographics

A complete medical history, including history of symptoms associated with Rett syndrome, will be performed at Screening to document all current medical conditions, and previous major medical events and conditions. For subjects who were already receiving care for Rett syndrome at the study site, summary documents from the medical record (such as clinician's summaries) should be available as source documentation of major medical conditions or events (e.g., surgeries) within the last two years. For subjects who were not a part of the clinical site's practice, the study team should obtain summary medical records from their other providers in preparation of the screening visit.

The Rett Syndrome Clinical Severity Scale (RTT-CSS)

The RTT-CSS has been evaluated in over 1200 RTT children, adolescents and adults enrolled in the NIH-sponsored Natural History of Rare Diseases Project. This scale has been used as a measure of severity as reported in studies of genotype/phenotype correlations and epilepsy in RTT (Glaze et al. 2010) and was evaluated as an outcome measure in the Neu-2566-RETT-001 study of trofinetide in adolescent and adults with RTT. The scale was derived from that reported in Amir et al. (2000) and Monros et al. (2001).

The RTT-CSS is a clinician-completed rating scale that measures the severity of core symptoms of RTT. The CSS consists of 13 items, 3 of which measure historical or static characteristics (age of onset of regression, age of onset of stereotypes, head growth) and 10 of which measure current function (somatic growth, independent sitting, ambulation, hand use, scoliosis, language, nonverbal communication, respiratory dysfunction, autonomic symptoms, and epilepsy/seizures) at the time of assessment, i.e., during the study visit. All items are scored during a clinical interview and examination by the Investigator or qualified designee using either a 4- or 5-point Likert scale. Individual subscale scores and a total score are calculated.

The RTT-CSS will be administered at Screening only.

Efficacy Assessments

All assessments will be administered in a standardized manner. Clinician completed measures will be completed by trained practitioners. Caregiver completed assessments will be reviewed by study personnel and caregivers will receive standardized training and guidance on how to complete the measures. To the extent possible, all efforts should be made to maintain the same caregiver informant or clinician rater (as applicable) across visits for a single subject.

Rett Syndrome Behaviour Questionnaire (RSBQ)

The Rett Syndrome Behaviour Questionnaire (RSBQ) is a 45 item caregiver-completed rating scale assessing a wide range of neurobehavioral symptoms known to be impaired in RTT (Mount et al. 2002). The RSBQ is a well-validated instrument that has been used in the Phase 2 study, Neu-2566-RETT-002, as well as in other observational and interventional studies in RTT (Glaze et al. 2019; Khwaja et al. 2014; O'Leary et al. 2018). The RSBQ has been correlated with functioning and quality of life and has been characterized and validated across a range of ages and genetic variations in RTT (Cianfaglione et al. 2015, 2016; Robertson et al. 2006; Barnes et al. 2015). The scale includes 45 items, 39 of them grouped into 8 subscales, whose ratings reflect the severity and frequency of symptoms. The caregiver rates items as “0” (Not True), “1” (somewhat or sometimes true) or “2” (very true). The eight subscales include:
1. General Mood
2. Breathing problems
3. Hand Behavior
4. Face movements
5. Body rocking/expressionless face
6. Night-time behaviors
7. Fear/anxiety
8. Walking/standing

The RSBQ will be administered at Screening, Baseline (Visit 2), and Visits 3, 4, and 5/ET. As much as possible, caregiver raters will remain the same throughout the study. At the start of the study all caregiver raters will be required to complete a standardized training on how to complete the scale.

Clinical Global Impression-Improvement (CGI-I) and Clinical Global Impression-Severity (CGI-S)

The CGI-I scale will be administered at Visits 3, 4, and 5/ET. Completion of this scale requires the clinician to rate how much the subject's illness has improved or worsened relative to a baseline state. A 7-point scale is used from 1=very much improved, 2=much improved, 3=minimally improved, 4=no change, 5=minimally worse, 6=much worse, 7=very much worse.

The CGI-S assessment will be made at Screening, Baseline, and Visits 3, 4, and 5/ET. The CGI-S is a 7-point scale that requires the clinician to rate the severity of the subject's illness at the time of assessment, relative to the clinician's experience with subjects who have the same diagnosis. Considering total clinical experience, a subject is assessed on severity of illness at the time of rating: 1, normal, not at all ill; 2, borderline ill; 3, mildly ill; 4, moderately ill; 5, markedly ill; 6, severely ill; or 7, extremely ill. In this study, the illness being assessed is Rett syndrome as a whole. Following best practice, the CGI-S and CGI-I ratings for the study will be assessed using RTT-specific anchors across major symptom areas in the same manner as in the Phase 2 studies (Neul et al. 2015; Busner and Targum 2007; Glaze et al. 2017; Glaze et al. 2019).

Communication and Symbolic Behavior Scales Developmental Profile Infant-Toddler (CSBS-DP-IT) Checklist

The Communication and Symbolic Behavior Scales-Developmental Profile™ (CSBS-DP) is standardized screening scale for assessing communication and pre-linguistic skills in young children 12-24 months (Wetherby et al. 2002) and can be used with older children with developmental delay (Anagnostou et al. 2015; Urbanowicz et al. 2016). The CSBS-DP includes a suite of three separate measures: The Infant-Toddler Checklist, a follow-up Caregiver Questionnaire and a Behavior Sample. In this study only the Infant-Toddler (CSBS-DP-IT) Checklist will be used.

Given the limited communication abilities of individuals with Rett Syndrome, the CSBS-DP-IT Checklist was assessed and a subset of items were found to be appropriate for assessing communication skills of individuals with Rett syndrome 8 to 19 years of age (Urbanowicz et al. 2016). The CSBS-DP-IT was assessed in a Phase 2 trial of mecasermin (recombinant human IGF-1) a compound related to trofinetide, in children with Rett syndrome 2 to 10 years of age (O'Leary et al. 2018). In that study, the first 16 items were completed, which allowed for calculation of the Social composite score. The CSBS-DP-IT demonstrated evidence of benefit in subjects in the active treatment group compared to those in the placebo treatment group (O'Leary et al. 2018). The CSBS-DP Social composite score has also shown evidence of sensitivity to change in behavioral intervention studies in other developmental disorders (e.g., Wetherby et al. 2014; Anagnostou et al. 2015).

The CSBS-DP-IT Checklist is a 24-item rating scale completed by the caregiver. Each item is scored using a three-level rating of frequency: “not yet”, “sometimes” and “often”. Three composite scores assessing 7 skill areas can be calculated: 1) Social Composite (including Emotion and Eye Gaze, Communication Rate and Function, and Gestures); 2) Speech Composite (including Sounds and Words); 3) Symbolic Composite (including Understanding and Object Use).

All 24 items on the Infant-Toddler Checklist are to be completed by the caregiver. The question after item 24 (“Do you have any concerns about your child's development?”) is not to be completed. At each administration, the study staff will review the instructions and scoring rubric with the caregiver. The Social Composite raw score, comprised of items 1 to 13, is used for the key secondary endpoint. The CSBS-DP-IT Checklist will be administered at Baseline, Visit 3, Visit 4, and Visit 5/ET.

Impact of Childhood Neurologic Disability Scale (ICND)

The Impact of Childhood Neurologic Disability Scale (ICND) was developed to evaluate the impact that a child's condition has on the child's and the family's everyday life at the present time and during the previous 3 months (Camfield et al. 2003). The parent or other caregiver evaluates the effect of four conditions or health problems on 11 aspects of the child's or the family's life as “A lot”, “Some”, “A little”, “Not at all”, or “Does not apply”. The four conditions or health problems are 1) inattentiveness, impulsivity, or mood, 2) ability to think and remember, 3) neurologic or physical limitations, and 4) epilepsy.

The caregiver then rates overall quality of life of the subject by responding to the following:
“Please rate your child's overall ‘Quality of Life’ on the scale below. Choose the number which you feel is best and circle it”. The choices range from 1 (“Poor”) to 6 (“Excellent”). The assessment will be administered at Baseline and Visit 5/ET.

Rett Syndrome Clinician Rating Scale of Hand Function (RTT-HF)

The Rett Syndrome Clinician Rating of Hand Function is a clinician completed clinical assessment of the subject's ability to use her hands for functional purposes (such as reaching for and grasping objects, self-feeding or drawing). The assessment is made on an 8-point Likert scale (0-7) with 0 denoting normal functioning and 7 the most severe impairment.

This rating is a further development of the RTT-DSC Hand Use Rating used in Study Neu-2566-RETT-002. The assessment will be administered at Baseline, Visit 3, Visit 4, and Visit 5/ET.

Rett Syndrome Clinician Rating Scale of Ambulation and Gross Motor Skills (RTT-AMB)

The Rett Syndrome Clinician Rating of Ambulation and Gross Motor Skills is a clinician completed clinical assessment of the subject's ability to sit, stand, and ambulate (e.g., walking, running, climbing stairs). The assessment is made on an 8-point Likert scale (0-7) with 0 denoting normal functioning and 7 the most severe impairment. This rating is a further development of the RTT-DSC Ambulation Rating used in Study Neu-2566-RETT-002. The assessment will be administered at Baseline, Visit 3, Visit 4, and Visit 5/ET.

Rett Syndrome Clinician Rating Scale of Ability to Communicate Choices (RTT-COMC)

The Rett Syndrome Clinician Rating of the Ability to Communicate Choices is a clinician completed clinical assessment of the subject's ability to communicate her choices or preferences, which can include the use of nonverbal means such as eye contact or gestures.

The assessment is made on an 8-point Likert scale (0-7) with 0 denoting normal functioning and 7 the most severe impairment. This rating is a further development of the RTT-DSC Language/Communication Rating used in Study Neu-2566-RETT-002. The assessment will be administered at Baseline, Visit 3, Visit 4, and Visit 5/ET.

Rett Syndrome Clinician Rating Scale of Verbal Communication (RTT-VCOM)

The Rett Syndrome Clinician Rating of Verbal Communication is a clinician completed clinical assessment of the subject's ability to communicate verbally (e.g., words and phrases).

The assessment is made on an 8-point Likert scale (0-7) with 0 denoting normal functioning and 7 the most severe impairment. This rating is a further development of the RTT-DSC Language/Communication Rating used in Study Neu-2566-RETT-002. The assessment will be administered at Baseline, Visit 3, Visit 4, and Visit 5/ET.

Rett Syndrome Caregiver Burden Inventory (RTT-CBI)

The RTT-CBI is a syndrome-specific, caregiver-completed questionnaire that is based on the Caregiver Burden Inventory designed for Alzheimer's disease (Lane et al. 2017; Novak and Guest 1989). The scale is intended to directly address caregiver burden and indirectly assess the significance of treatment effects on function in the context of activities of daily living. Caregivers rate how often a given statement describes their feeling or experience. Frequency ratings are on a 5-point Likert scale including: 0-never; 1-rarely; 2-sometimes; 3-frequently and 4-nearly always. As in the original Caregiver Burden Inventory, the RTT-CBI has 24 negatively worded items (items 1 through 24) yielding a total score up to 96. The RTT-CBI also includes 2 positively worded items (items 25 and 26) that comprise the Optimism Index (Lane et al. 2017). In this study, as in the 2 previous studies of trofinetide in Rett syndrome, the total score is defined as the total Burden score (items 1-24). The RTT-CBI will be completed at Baseline and Visit 5/ET.

Safety Assessments

Physical Examination

A general physical examination will be conducted at Screening, Baseline, Visit 3, Visit 4, and Visit 5/ET. The physical exam procedures will include the following organ systems:
Neurological
Head, ears, eyes, nose, and throat
Skin
Cardiovascular
Respiratory
Abdomen
Genitourinary (optional)
Musculoskeletal
Vital Signs

Vital signs will include body temperature, resting respiration rate, sitting systolic and diastolic blood pressure, and pulse rate. The sitting blood pressure should be measured after the subject has been sitting for ≥3 minutes. Vital signs to be measured at Screening, Baseline, Visit 3, Visit 4, and Visit 5/ET.

Height, Weight, and Body Mass Index

Height will be measured at Baseline and at Visit 5/ET.

Weight will be measured at Screening, Baseline, and Visit 5/ET.

Body mass index will be calculated using the following formula:

Weight (kg)/[height (m)]².

Electrocardiograms

All 12-lead ECGs will be complete, standardized recordings. ECGs will be completed in triplicate at Visit 1 (Screening), at Visit 2 (Baseline) both before dosing and 2-3 hours after dosing, and at Visit 5/ET (Week 12/EOT). A single ECG will be completed at Visit 3 (Week 2) and Visit 4 (Week 6).

The subject must rest for 5 minutes in a supine position before the ECG is obtained. ECG tracings (paper or electronic) will be reviewed and interpreted by a qualified clinician for prolongation of the QTcF interval and for other cardiac irregularities. ECG tracings and results (ventricular rate, PR, QRS, QT, QTcF and QTcB intervals) will be included in the subject's study records.

ECGs will also be read by a qualified central reader. The central reading will be the reading that is entered in the database. The results from the reports from the central reader will also be reviewed by the Investigator. At Screening and Baseline (before dosing) the average QTcF interval of all legible ECGs will be used to determine eligibility. At Baseline, the Investigator will use the machine readings to determine eligibility if the central reading has not yet been received.

If the mean QTcF value from the set of ECGs done at Screening is prolonged due to an identifiable cause, and it is medically appropriate to address that cause, a repeat set of triplicate ECGs may be performed during the Screening period and before the Baseline visit with the agreement of the Medical Monitor. In this case, the repeat ECGs will be used in determination of subject eligibility.

Post-Baseline QTcF Interval Stopping Criteria

In the event that a post-randomization QTcF duration of 2500 ms or an increase of ≥60 ms compared to the average QTcF interval at Baseline (before dosing) is observed, study drug administration is to be discontinued.

Laboratory Evaluations

Laboratory evaluations will be completed according to the schedule presented in Table S-2 and procedures detailed in the laboratory manual. Additional safety testing may be performed at the discretion of the Investigator or designee. Laboratory testing may be repeated during the Screening period with agreement of the Medical Monitor if the Investigator suspects that a laboratory abnormality is a temporary or reversible finding.

Clinical laboratory sample collection is not required to be completed under fasting conditions. The laboratory evaluations will include the following:
Clinical chemistry serum tests
   Sodium (Na), potassium (K), chloride (Cl), phosphorus (P), calcium (Ca), magnesium (Mg), carbon dioxide (CO2), blood urea nitrogen (BUN), creatinine (CR), uric acid
      Mg should only be performed at Visit 1 (Screening)
   Alanine aminotransferase (ALT), aspartate aminotransferase (AST), gamma-glutamyl transpeptidase (GGT), alkaline phosphatase (ALP), total bilirubin (TBIL), lactate dehydrogenase (LDH)
   HbA1c
      HbA1c should only be performed at Visit 1 (Screening)
   Glucose
   Albumin (ALB), total protein
   Thyroid stimulating hormone (TSH), free T3, and free T4
      Thyroid function tests will be performed at Visit 1 (Screening), Visit 2 (Baseline), and Visit 5/ET
Pregnancy test
   A serum pregnancy test should be performed at the designated visits (Table S-2) for subjects of childbearing potential
Hematology tests
   Complete blood count (CBC) including:
      White blood cell (WBC) count
      Complete differential (relative and absolute)
      Hematocrit (Hct), hemoglobin, red blood cells (RBC), platelets
      Reticulocyte count
Urinalysis
   Blood, RBCs, WBCs, protein, glucose, ketones, specific gravity, pH
   Reasonable efforts should be made to collect a urine sample from all subjects.

When collection of a urine sample proves impractical or impossible (e.g., because the subject is incontinent or unable to cooperate), failure to collect a urine sample should be recorded in the subject's eCRF, and will not be considered a protocol deviation.

For the 2 days before clinic visits as well as the morning of the clinic visit, the dates and times of trofinetide dosing, concomitant medication dosing, and meals should be recorded in the caregiver diary.

The caregiver diary will be completed and collected on an ongoing basis throughout the study from Screening to Visit 5/ET. The clinician will verbally ask about AEs (see Section 7.3.1), review the events recorded in the diary with the caregiver and will make a clinical evaluation, including an evaluation of whether an adverse event should be reported.

Pharmacokinetic Assessments

Pharmacokinetic blood samples will be collected for measurement of whole blood concentrations of trofinetide and possible metabolites identified.

PK blood samples will be collected at 5 timepoints for trofinetide concentration measurements at the Baseline visit (both before dosing and approximately 2-3 hours after dosing) and at Visit 3, Visit 4, and Visit 5/ET in accordance with the sampling schedule outlined below (Table 6-1).

The second PK sample to be taken at the Baseline visit (Visit 2), approximately 2-3 hours after dosing, should take place as soon as possible after the postdose ECG is performed.

PK samples at Visits 3, 4, and 5 should be collected at one of the following time intervals:
2-3 hours after dosing
3-7 hours after dosing
7-11 hours after dosing

Over the duration of the study, every effort should be made to collect the PK samples across Visits 3, 4, and 5 at each one of the different time intervals (2-3 hours after dosing, 3-7 hours after dosing, and 7-11 hours after dosing). If samples are taken within the same time interval across visits, every effort should be made to collect the samples at different times within the specified time interval.

Pharmacokinetic samples will also be collected, if possible, at any ET visit or the visit immediately following any SAE or following any AE leading to discontinuation, even if it is an unscheduled visit.

For all PK samples (scheduled and unscheduled) the dates and times of administration of the last 3 doses of study drug, the dates and times of the meals closest to the times those doses were given, as well as the date and time of the sample draw should be recorded. For samples collected from subjects who experience any SAE or experience an AE leading to discontinuation, the date and time of the last dose of study drug prior to the SAE or AE should also be recorded.

Specimen Preparation, Handling, Storage, and Shipment

PK blood samples may be collected from a cannula port or via venipuncture. Pre-prepared PK sampling tubes will be provided to each site within the lab visit kits for collection and storage of PK samples. Blood samples will be processed for determination of trofinetide whole blood concentrations (and of concentrations of possible metabolites identified). At each time point, blood will be collected, processed as appropriate, and samples will be shipped to the central laboratory for storage and to the bioanalytical laboratory for analysis. A laboratory manual will be provided for sample processing, storage, and shipping procedures.

When possible, an additional PK sample will be collected from subjects who experience any SAE or experience an AE leading to discontinuation as soon as possible after the occurrence of that event.

Identification of Biomarkers of Response to Trofinetide in Rett Syndrome

Subjects for whom separate informed consent for the identification of biomarkers of response to trofinetide is provided (where local regulations permit) will have blood drawn and stored for future investigations. Blood samples will be taken at Baseline (before dosing) and at Visit 5/ET. Participation in the effort to identify biomarkers is an optional component of the study requiring a separate informed consent, which may be obtained at any time during the study. If consent is obtained after Baseline, only the sample at Visit 5/ET will be taken. Blood samples will be used to investigate differences between responders and non-responders in both trofinetide-treated and placebo-treated subjects in RNA transcripts (transcriptomics), proteins (proteomics), and metabolites (metabolomics). Unbiased analyses and targeted analyses will test candidate molecular pathways based on the available knowledge of RTT and trofinetide at the time of the investigation (Erhart et al. 2016; Shovlin and Tropea 2018; West et al 2014; Buchovecky et al. 2013). The analysis of biomarkers does not include any DNA, genomic, or genetic testing or analysis.

Stored samples and relevant clinical data will be made non-identifiable after the clinical study report has been issued. Any personal identifiers will be removed, and each study subject identifier will be replaced with a new number to limit the possibility of linking genetic data to a subject's identity.

### Adverse Events

Specification of Safety Parameters

Definition of Adverse Event

An AE is defined as “any untoward medical occurrence in a patient or clinical study participant, temporally associated with the use of study drug, whether or not considered related to study drug”.

An AE can therefore be any unfavorable and unintended sign (e.g., an abnormal laboratory finding), symptom, or disease temporally associated with the use of a drug, without any judgment about causality or seriousness. An AE can arise from any use of the drug (e.g., off-label use, use in combination with another drug) and from any route of administration, formulation, or dose, including an overdose.

A suspected adverse reaction is any AE for which there is a reasonable possibility that the drug caused the AE. AEs do not include the following.
Stable or intermittent chronic conditions (such as myopia requiring eyeglasses) that are present prior to Baseline and do not worsen during the study
Medical or surgical procedures (e.g., surgery, endoscopy, tooth extraction, transfusion). The condition that leads to the procedure is an AE if not present at Baseline
Overdose of concomitant medication without any signs or symptoms unless the subject is hospitalized for observation
Hospitalization for elective surgery planned prior to study (situation where an untoward medical occurrence has not occurred)
Pregnancy will not be considered an AE, but if it occurs, it will be reported on a pregnancy form

For subjects who enroll into the open-label extension study, AEs will be recorded from the time informed consent is obtained in the present study until the first dose of study drug in the open-label study. For subjects who discontinue from the study or do not enroll into the open-label extension, AEs will be recorded from the time informed consent is obtained until 30 days after the last dose of study drug.

Definition of Serious Adverse Event

In addition to the severity rating, each AE will be classified by the Investigator as “serious” or “not serious.” The seriousness of an event will be defined according to the applicable regulations and generally refers to the outcome of an event. An SAE is one that meets one or more of the following:
Is fatal
Is immediately life threatening
Results in disability or permanent damage
Requires hospitalization
Prolongs existing hospitalization
Is a congenital anomaly or birth defect (in an offspring)
Is medically significant

Definition of Life Threatening

A life-threatening event places the subject at immediate risk of death from the event as it occurred. This does not include an AE, which, had it occurred in a more severe form, might have caused death.

Definition of Hospitalization

Hospitalization is defined by the Sponsor as a full admission to the hospital for diagnosis and treatment. This includes prolongation of an existing inpatient hospitalization. Examples of visits to a hospital facility that do not meet the serious criteria for hospitalization include:
Emergency room visits (that do not result in a full hospital admission)
Outpatient surgery
Preplanned or elective procedures
Protocol procedures
Social hospitalization, defined as admission to the hospital as a result of inadequate family support or care at the subject's primary residence

Definition of Disability or Permanent Damage

Disability is defined as a persistent or significant incapacity or substantial disruption of the ability to conduct normal life functions.

Definition of Medically Significant

Important medical events (medically significant events) that may not result in death, be life threatening, or require hospitalization may be considered to be an SAE when, based upon appropriate medical judgment, they may jeopardize the subject or may require medical or surgical intervention to prevent one of the outcomes listed in this definition. Examples of such events are intensive treatment in an emergency room or at home for allergic bronchospasm, blood dyscrasias, or convulsions that do not result in hospitalization or development of drug dependency or drug abuse.

An SAE may also include any other event that the Investigator or Medical Monitor judges to be serious or that suggests a significant hazard, contraindication, side effect, or precaution.

Classification of an Adverse Event

Severity of Event

The severity of each AE will be graded on a 3-point scale and reported in detail as indicated on the eCRF:
Mild: awareness of sign or symptom but easily tolerated, causing minimal discomfort, and not interfering with normal everyday activities
Moderate: sufficiently discomforting to interfere with normal everyday activities
Severe: incapacitating and/or preventing normal everyday activities

Relationship to Study Drug

The causality of each AE should be assessed and classified by the Investigator as “related” or “not related.” An event is considered related if there is a reasonable possibility that the event may have been caused by the product under investigation (i.e., there are facts, evidence, or arguments to suggest possible causation).

Consider the Following when Assessing Causality:
Temporal associations between the agent and the event
Response to cessation (de-challenge) or re-challenge
Compatibility with known class effect
Known effects of concomitant medications
Pre-existing risk factors
A plausible mechanism
Concurrent illnesses

Duration

The start and stop dates for AEs will be recorded using the following criteria:
Start: Date of the first episode of the AE or date of significant sustained worsening in severity
Stop: Date when AE either ceased permanently or changed in severity

Frequency

The frequency of the AE should be indicated according to the following definitions:
Single: Experienced once, without recurrence
Recurrent: More than one discrete episode with the same severity

Action Taken with Study Drug
Dose not changed: No change in study drug
Drug interrupted: Study drug temporarily stopped
Drug withdrawn: Study drug discontinued permanently

Therapy
None: No new treatment instituted
Medication: New treatment initiated as a direct result of AE
Other: Other action required

Outcome
Recovered/resolved: Recovered or resolved
Recovered/resolved with sequelae: Recovered or resolved with sequelae
Not recovered/not resolved: Not recovered or not resolved
Fatal: Death due to an AE
Unknown: Unknown

Seriousness
Not serious
Serious

Definition of Unexpectedness

An AE, the nature or severity of which is not consistent with the information provided in the Reference Safety Information section of the current trofinetide Investigator's brochure.

Time Period and Frequency for Event Assessment and Follow-Up

Adverse events will be recorded from the time informed consent is obtained through the safety follow-up period. All AEs must be either resolved or stable at the end of the safety follow-up period. If ongoing at the end of the safety follow-up period, the subject should be referred for appropriate treatment.

In the event that a subject discontinues and has an ongoing AE at the time of discontinuation or is withdrawn from the study because of an AE, the subject should be followed and treated by the Investigator until the AE has resolved, stabilized, or a new chronic baseline has been established.

Adverse Event Reporting

The Investigator must record all observed AEs and all reported AEs. At each visit, the Investigator should ask the subject a nonspecific question (e.g., “Have you noticed anything different since your last visit?”) to assess whether any AEs have been experienced since the last report or visit.

Note that any use of medication (and specifically any newly prescribed medication) during the course of a study may indicate the occurrence of an AE that may need to be recorded on both the AE and the concomitant medication page.

All AEs, serious and not serious, will be recorded on the AE eCRF page using appropriate medical terminology. Severity and relationship to study drug will be assessed by the Investigator. When possible, clinical AEs should be described by diagnosis and not by symptoms (e.g., “cold” or “seasonal allergies” instead of “runny nose”).

All AEs, whether or not related to the study drug, must be fully and completely documented on the AE eCRF and in the subject's notes.

Serious Adverse Event Reporting

The reporting of SAEs by the Sponsor or designee to the regulatory authorities is a regulatory requirement. Each regulatory authority has established a timetable for reporting SAEs based upon established criteria.

Serious AEs must be reported within 24 hours of discovery to the Sponsor or its designee; use the appropriate form for initial and/or follow-up reporting.

At a minimum, events identified by the Sponsor to require expedited reporting as serious, unexpected, and related to study drug must be brought to the attention of the responsible Institutional Review Board/Ethics Committee (IRB/EC), as per applicable regulations. These will be provided by the Sponsor after their assessment. For European Union member states, the Sponsor or its designee will provide reports of suspected unexpected serious adverse reactions (SUSARs) directly to the ECs, as required by local legislation. In all other countries, it is the Investigator's responsibility to provide these expedited reports to the responsible IRB/EC. It is also the Investigator's responsibility to notify the responsible IRB/EC regarding any new and significant safety information.

When an SAE occurs, Investigators will review all documentation related to the event and will complete the paper SAE form (for initial and/or follow-up information) and fax or email (within 24 hours of discovery) to the contact information provided on the SAE form. Subjects will be followed through the safety follow-up period for 30 days after last dose of study drug for any SAEs and/or other reportable information until such events have resolved or the Investigator, in conjunction with the Sponsor, deems them to be chronic or stable. In the event of any SAE (other than death), the study subject will be instructed to contact the Investigator (or designee) using the telephone number provided in the ICF. All subjects experiencing an SAE will be seen by the Investigator or designee as soon as is feasible following the report of the SAE.

Serious AEs occurring after the study follow-up period (i.e., 30 days after last dose of study drug) should be reported if in the judgment of the Investigator there is “a reasonable possibility” that the event may have been caused by the product. SAEs should also be reported to the IRB/EC according to local regulations.

Reporting of Pregnancy

Any subject who becomes pregnant during the study (with or without AEs) must be withdrawn from the study and the pregnancy must be reported on the Pregnancy form within 24 hours of discovery to the Sponsor or its designee. Any subject who becomes pregnant during the study will be followed through the pregnancy outcome.

Any AEs that are the consequence of pregnancy and which meet the criteria for serious should also be reported via the SAE form.

Reporting Paternal Drug Exposure

Paternal drug exposure is defined as a father's exposure to a medicinal product before or during his partner's pregnancy. Any paternal drug exposure cases must be reported to the Sponsor within 24 hours of discovery via the Pregnancy form. Any AEs that are the consequence of paternal drug exposure and which meet the criteria for serious must also be reported to the Sponsor within 24 hours of discovery via the SAE form. Since no males are enrolling in this study, paternal drug exposure would occur only if a male who was not a study subject ingested study drug.

Reporting of Overdose

An overdose is a deliberate or inadvertent administration of a treatment at a dose higher than the maximum recommended dose per protocol. It must be reported to the Sponsor or designee on the Overdose form within 24 hours of discovery. In addition, all events of overdose are to be captured as protocol deviations.

### Clinical Monitoring

Clinical site monitoring is conducted to ensure that the rights and well-being of human subjects are protected, that the reported study data are accurate, complete, and verifiable, and that the conduct of the study is in compliance with the currently approved protocol and amendment(s) as applicable, with GCP, and with applicable regulatory requirements. Details of the study site monitoring process are described in a separate clinical monitoring plan document.

### Statistical Methods and Data Analysis

Statistical Hypotheses

The co-primary endpoints are change from Baseline to Week 12 in RSBQ total score and CGI-I score at Week 12. Let ΔRSBQ and ΔCGI-I be the difference between the trofinetide and placebo groups in the mean change from Baseline to Week 12 in RSBQ total score and in the mean CGI-I score at Week 12 respectively.

RSBQ: The null hypothesis is: ΔRSBQ=0 and the alternative hypothesis is: ΔRSBQ≠0.

CGI-I: The null hypothesis is: ΔCGI-I=0 and the alternative hypothesis is: ΔCGI-I≠0.

Sample Size Determination

The sample size calculation was performed for the co-primary endpoints as a family of two hypothesis tests at an overall two-sided significance level of 0.05. A total sample size of 174 evaluable subjects in a 1:1 ratio to trofinetide or placebo was estimated to provide at least 90% power for the hypothesis testing family assuming the following treatment differences (SD) estimated from Phase 2 study data: −4.4 (8) for the mean change from Baseline to Week 12 in the RSBQ total score and −0.5 (0.7) for the CGI-I mean score at Week 12.

The sample size of 174 evaluable subjects will provide at least 95% power at a two-sided significance level of 0.05 for each individual hypothesis test within the family. Trofinetide will be superior to placebo if both hypothesis tests within the family are shown to be statistically significant at 0.05. Based on data from Phase 2 study, the correlation between the RSBQ total score and the CGI-I score is low, so the measures are assumed to be independent. Therefore, the overall power to detect a treatment difference on both of the co-primary endpoints will be at least 90% (0.952).

Adjusting for an anticipated discontinuation rate of up to 5%, approximately 184 subjects will be randomized in a 1:1 ratio to trofinetide or placebo.

Subject Populations for Analysis

The following populations will be defined and used in the analysis:

The Safety Analysis Set will consist of all randomized subjects who received at least one dose of study medication. The Safety Analysis Set will be analyzed according to the actual treatment received. The Safety Analysis Set will be used for the safety analysis.

The Full Analysis Set (FAS) will consist of all randomized subjects who received at least one dose of study medication and who have both a Baseline value and at least one post-Baseline value for the RSBQ total score or who have at least one post-Baseline value for the CGI-I score. The FAS will be analyzed according to the treatment they were assigned regardless of the actual treatment received. The Full Analysis Set will be used for the efficacy analysis.

The Per-protocol (PP) Analysis Set will consist of the subjects in the Full Analysis Set who did not have a major protocol violation that would affect interpretation of the efficacy data. The Per-protocol Analysis Set will be defined prior to study unblinding. The Per-protocol Analysis Set will be analyzed according to the actual treatment received. The Per-protocol Analysis Set will be used for the sensitivity analysis.

The Pharmacokinetic Analysis Set will consist of subjects in the Safety Analysis Set with at least one measurable trofinetide whole blood concentration.

Statistical Analyses

General Approach

Unless stated otherwise, all statistical tests will be 2-sided using a 5% significance level, leading to 95% (2-sided) confidence intervals. Trofinetide will be superior to placebo if both of the hypothesis tests with respect to the co-primary endpoints are shown to be statistically significant.

Continuous measurement results will be reported using the number of subjects with data values, mean, standard error of the mean, median, standard deviation, minimum, and maximum. For each categorical outcome, the number and percentage of subjects in each category will be reported.

A hierarchical approach will be used to control for the multiple endpoints (co-primary and secondary).

Primary Analyses

The co-primary efficacy endpoints will be analyzed using a mixed model for repeated measures (MMRM). An unstructured covariance matrix will be used and the Kenward-Roger approximation will be used to adjust the denominator degrees of freedom. The treatment comparisons will be based on the difference in least squares means at Week 12. For the change from Baseline in the RSBQ total score, the MMRM model will include effects for treatment group, age group (5-10 years old, 11-15 years old, and 16-20 years old), visit, Baseline RSBQ total score, and interactions for treatment group by visit and Baseline RSBQ total score by visit.

For the CGI-I score, the MMRM model will include effects for treatment group, age group (5-10 years old, 11-15 years old, and 16-20 years old), visit, Baseline RSBQ severity (<35 total score and >35 total score), Baseline CGI-S score, and interactions for treatment group by visit and Baseline CGI-S score by visit.

Sensitivity analyses will be performed to assess the impact of missing data, including analyses based on a missing not at random assumption.

Secondary Analyses

The key secondary endpoint will be analyzed using a MMRM method with effects for treatment group, age group (5-10 years old, 11-15 years old, and 16-20 years old), visit, Baseline RSBQ severity (<35 total score and ≥35 total score), Baseline CSBS-DP-IT Social score, and interactions for treatment group by visit and Baseline CSBS-DP-IT Social score by visit. An unstructured covariance matrix will be used and the Kenward-Roger approximation will be used to adjust the denominator degrees of freedom. The treatment comparisons will be based on the difference in least squares means at Week 12.

For the other secondary endpoints that are assessed at multiple post-Baseline visits, the change from Baseline will be analyzed using a MMRM analysis similar to those described above for the co-primary endpoints. The MMRM model will include effects for treatment group, age group (5-10 years old, 11-15 years old, and 16-20 years old), visit, Baseline RSBQ severity (<35 total score and >35 total score), Baseline score, and interactions for treatment group by visit and Baseline score by visit.

For the other secondary endpoints that are assessed at a single post-Baseline visit (i.e. Week 12 only), the change from Baseline will be analyzed using an analysis of covariance (ANCOVA) model with effects for treatment group, age group (5-10 years old, 11-15 years old, and 16-20 years old), Baseline RSBQ severity (<35 total score and >35 total score), and Baseline score.

Safety Analyses

Safety results will be summarized by treatment group using descriptive statistics. No formal statistical testing will be performed for any of the safety endpoints. Adverse events will be classified into standard terminology using the Medical Dictionary for Regulatory Activities (MedDRA). Treatment-emergent adverse events (TEAEs), TEAEs leading to discontinuation, TEAEs related to study drug, TEAEs by maximum severity, fatal TEAEs, SAEs, and SAEs related to study drug will all be summarized.

Descriptive statistics for ECG, vital signs and weight, and clinical laboratory parameters, including changes from Baseline, will be tabulated by timepoint. Additionally, categorical analyses will be conducted on the incidence of subjects with prolonged QTc intervals and changes in QTc intervals in accordance with International Council on Harmonisation (ICH) guidelines.

Pharmacokinetic Analyses

Pharmacokinetic (PK) and efficacy (PD) measures will be collected from all subjects at the Baseline (Week 0) visit before dosing, at the Baseline (Week 0) visit after dosing, and after dosing at Weeks 2, 6, and 12/EOT.

Whole blood concentration and possible metabolites data for trofinetide will be listed and summarized using descriptive statistics. If data allow, population PK and PK/PD analyses

will be performed to further characterize the PK profile and exposure response relationship of trofinetide using measures of safety and efficacy parameters. Trofinetide whole blood concentration data will remain blinded until the unblinding of the clinical database at the end of the study. The details of the PK and PK/PD analysis will be presented in a separate Clinical.

Data and Safety Monitoring Board

An independent Data and Safety Monitoring Board (DSMB) will review safety information on a regular basis throughout the study. The DSMB will be independent of the Sponsor and will be empowered to recommend stopping the study due to safety concerns. The DSMB may review blinded, unblinded, or partially unblinded data, but the Sponsor and the Investigators will remain blinded to the data provided to the DSMB until the official unblinding of the database at the completion of the study. The membership, activities, responsibilities, and frequency of meetings will be described separately in the DSMB charter.

Measures to Minimize Bias

Eligible subjects will be randomized into one of two treatment groups (trofinetide or placebo) in a 1:1 ratio using an interactive response technology (IRT) system. The randomization will be stratified by age group (5-10 years old, 11-15 years old, and 16-20 years old) and Baseline RSBQ severity (<35 total score and >35 total score). The assignments will be based on a pre-generated permuted-block randomization schedule. Blinding will be assured by restricting access of Investigators and Sponsor personnel and/or designee to the treatment codes and providing identical packaging for the trofinetide and placebo treatments.

Breaking the Study Blind/Subject Code

For the final analysis, the treatment codes for all subjects will be released to the Sponsor after all subjects have completed the study and the clinical database is locked. For DSMB safety reviews, the treatment codes will be released to an independent statistician/programmer to produce unblinded statistical outputs. The Sponsor and the Investigators will remain blinded.

Unblinding of individual treatment assignment during the study is discouraged. The Investigator may break the blind in the event of a medical emergency if it is considered necessary for the care of the subject. The Investigator should attempt whenever possible to contact the Medical Monitor before unblinding a subject's treatment to discuss the event. Lack of Medical Monitor contact does not preclude the Investigator from unblinding the subject. In an emergency situation, the subject's treatment assignment may be obtained by the Investigator from the IRT system. Details of the process to be followed are provided in a separate IRT manual. In the event that the IRT system is used to perform a code break, the Sponsor or designee will be notified immediately via an automated notification from the IRT system that an unblinding has occurred. The notification only alerts the Sponsor or designee that the unblinding occurred and does not include any information about the unblinded subject's treatment assignment.

### Ethical Considerations

The study will be conducted in compliance with the protocol, the Declaration of Helsinki, ICH GCP, and other applicable regulatory requirements (e.g., Serious Breach reporting, urgent safety measures, and EU GDPR).

The study will be performed in accordance with the US Health Insurance Portability and Accountability Act (HIPAA) regulations, US FDA GCP Regulations (US CFR 21 parts 50, 54, 56, and 312), and ICH guidance on GCP (E6) and clinical safety data management (E2A). In accordance with Directive 75/318/EEC, as amended by Directive 91/507/EEC, the final clinical study report will be signed by an Investigator and/or Coordinating Investigator who will be designated prior to the writing of the clinical study report. The Investigator or designee will provide the IRB/EC with all requisite material, including a copy of the protocol, informed consent, and any subject information or advertising materials.

The study will not be initiated until the IRB/EC provides written approval of the protocol and the informed consent and until approved documents have been obtained by the Investigator and copies received by the Sponsor. All amendments will be sent to the IRB/EC for information (minor amendment) or for submission (major amendment) before implementation. The Investigator will supply the IRB/EC and the Sponsor with appropriate reports on the progress of this study, including any necessary safety updates, in accordance with the applicable government regulations and in agreement with policy established by the Sponsor.

In accord with the provisions of the US CFR 21 part 50, and since this study involves greater than minimal risk but presents the prospect of direct benefit to all subjects enrolled, consent shall be obtained from an LAR, typically a guardian, or at least one parent, in accordance with local IRB requirements. Minors will be given the opportunity to assent to participation if and when they are considered capable of doing so by the PI and per local IRB requirements. Properly executed, informed consent/assent must be obtained from each LAR/subject prior to any screening procedures. The LAR is defined as ‘An individual or judicial or other body authorized under applicable law to consent on behalf of a prospective subject to the subject's participation in the procedures involved in the research’ (US CFR 21 part 50).

For subjects who are minors, written informed consent will be obtained from the LAR. For subjects who are not minors, written informed consent will be obtained from the LAR or the subject if deemed able by the Investigator. When consent is being provided by an LAR, subject assent for participation should be documented, when possible. Assent is the affirmative agreement to participate in the research of a minor or of an adult who does not have the capacity to consent. If written assent is not possible, verbal assent is allowed and must be documented. If subject assent is not possible, then the site must document rationale for not being able to provide written or documented subject assent.

If a subject's 18th birthday takes place during the study and the subject is deemed able to consent by the Investigator, the subject should sign the informed consent. Reconsenting should take place if required by and in accordance with IRB or EC policy and applicable local law.

The subject's caregiver must also provide informed consent regarding their participation in the study prior to participating in any study procedures.

The informed consent must, at a minimum, include the elements of consent described in the ICH guidance on GCP and the US CFR 21 part 50.25. A copy of the ICF planned for use will be reviewed by the Sponsor or designee for acceptability and must be submitted by the Investigator or designee together with the protocol, to the appropriate IRB/EC for review and approval prior to the start of the study at that investigational site. Consent forms must be in a language fully comprehensible to the LAR of the prospective subject. The Investigator must provide the Sponsor or designee with a copy of the IRB/EC letter approving the protocol and the ICF before the study drug supplies will be shipped and the study can be initiated.

The consent form must be revised if new information becomes available during the study that may be relevant to the subject. Any revision must be submitted to the appropriate IRB/EC for review and approval in advance of use.

### Example 3. OPEN LABEL EXTENSION

A 40-week, multicenter, open-label extension (OLE) study is planned to be conducted. Subjects who complete the preceding double-blind study (Example 2) will be eligible to enroll in the OLE. Legally acceptable representatives (LARs)/subjects must be consented prior to the procedures being performed at the Week 12/end of treatment (EOT) visit of the antecedent study. The Week 12/EOT visit of the antecedent study will serve as the Baseline visit of the present study. The data gathered at the Week 12/EOT visit of the antecedent study serves as the baseline data of the present study. The study will have two periods:
Treatment period: 40 weeks (Treatment with trofinetide is as described above in Example 2)
Safety follow-up period: 30 days

### Open-Label Treatment Period (40 Weeks

The Week 12/EOT visit of the antecedent study serves as the Baseline visit (Visit 1) of the present study. The first dose of study drug for the present study is intended to be administered at the study site after all Baseline assessments are completed. Since in most cases, the subject will have taken the last dose of study drug from the antecedent study on the morning of the Baseline visit for this study, the first dose of study drug for this study will then be administered as an evening dose after the Baseline visit has been completed. An electrocardiogram (ECG) must be performed 2-3 hours after the first dose and a PK sample will be taken upon completion of the ECG. Study drug must be discontinued in the event that a post-enrollment QTcF duration of ≥500 ms or an increase of ≥60 ms compared to the average QTcF interval at Baseline of the present study (before dosing) is observed.

If the Investigator judges it is too late in the day for the subject to take the first dose for the present study and then wait 2-3 hours for an ECG and drawing of a PK sample, the subject may return to the clinic the next morning to take the first dose and then wait 2-3 hours for an ECG and drawing of PK sample. Dosing is twice a day, once in the morning and once in the evening. The subject should not eat for 1 hour before dose administration and for 1 hour after dose administration. In addition to the study drug dispensed at the site at the Baseline visit, additional investigational product will be shipped directly to the subject or visiting nurse. Study drug shipment, return, and accountability will be performed in accordance with the drug distribution plan. Each site will also have a plan for distribution of drug to the subjects. Confirmation of delivery to the subject's home will be made by a visiting nurse at a home visit.

Subjects will return to the clinic for assessments at Week 2, Week 12, Week 26, and Week 40/early termination (ET).

### Safety Follow-Up Period (30 Days

Subjects will receive a follow-up telephone call to assess safety 30 days after the last dose of study drug.

### EQUIVALENTS

The disclosure may be embodied in other specific forms without departing from the spirit or essential characteristics thereof. The foregoing embodiments are therefore to be considered in all respects illustrative rather than limiting of the disclosure. Scope of the disclosure is thus indicated by the appended claims rather than by the foregoing description, and all changes that come within the meaning and range of equivalency of the claims are therefore intended to be embraced herein.

### Example 4

### Trofinetide Dose Selection in Pediatric Patients with Rett Syndrome

Objectives:

The objectives of the analyses were to: (i) Refine the population pharmacokinetic (PK) model using data from 5 Phase 1 studies and 4 Phase 2 studies; (ii) Use the updated population PK model to generate individual exposure measures (including area under the concentration-time curve from time 0 to 12 hours [AUC₀₋₁₂], maximum observed drug concentration [Cₘₐₓ], and average drug concentration during a dosing interval at steady state [C_{avg}]) for the patients with Rett syndrome; (iii) Develop exposure-response (E-R) models to characterize the relationships between primary efficacy endpoints (Clinical Global Impression-Improvement [CGI-I] and Rett Syndrome Behavior Questionnaire [RSBQ] scores) and trofinetide exposure from the data collected in Phase 2 Rett syndrome Studies; (iv) Perform stochastic simulations using the refined population PK model to predict trofinetide exposures following administration of the dosing regimens in the table below (Abbreviation: BID, twice daily). Compare the simulated exposure ranges to the target exposure range found to be associated with efficacy in the pediatric and adult patients with Rett syndrome; and (v) Use the population PK and E-R models to perform stochastic simulations to predict the response profile for younger pediatric patients (ages 2 to 5 years). Population pharmacokinetic studies are described in U.S. Department of Health and Human Services, Food and Drug Administration Guidance for Industry “Population Pharmacokinetics” (July 2019) and “Exposure-Response Relationships—Study Design, Data Analysis, and Regulatory Applications” (2003). These documents can be found at: https://www.fda.gov/drugs/guidance-compliance-regulatory-information/guidances-drugs. For example, the area under the concentration-time (AUC) full profile is a typical pharmacokinetic variable used to represent the average drug concentration over a time period. It is also a variable that can be used to compare exposure to a drug after multiple doses to single dose exposure. It is frequently useful to correlate long-term drug effects to steady-state AUC, as the effects usually reflect the daily exposure to drug following multiple dosing. Peak plasma concentrations (Cₘₐₓ) of a drug can be associated with a pharmacodynamics response, especially adverse events. The AUC, Cₘₐₓ, and other pharmacokinetic parameters of a drug in clinical use, e.g., trofinetide, in plasma or other biological fluids can be determined using standard methods known to one skilled in the art of clinical pharmacology

| | | |
|---|---|---|
| Body Weight (kg) | Dose | Total Daily Dose |

| | | | | |
|---|---|---|---|---|
| 12 to 20 | 30 mL | (6 g) BID | 60 mL | (12 g) |
| >20 to 35 | 40 mL | (8 g) BID | 80 mL | (16 g) |
| >35 to 50 | 50 mL | (10 g) BID | 100 mL | (20 g) |
| >50 | 60 mL | (12 g) BID | 120 mL | (24 g) |

Data Description:

Data for the population PK model development of trofinetide came from 5 Phase 1 studies and 4 Phase 2 studies, including patients with Rett syndrome and other diseases. A total of 3058 trofinetide concentration records collected in 290 subjects were available for potential use in the analysis. A blend of full-profile and sparse data from 94 healthy volunteers, 93 patients with Rett syndrome (adult and pediatric), 103 patients with other diseases was available for this analysis.

Exposure-response analyses of the CGI-I scores were performed on data from patients with Rett syndrome with available trofinetide exposure estimates, plus placebo patients. All of the patients included in this analysis had at least 1 CGI-I score measured after trofinetide or placebo dosing. Data from 52 adult patients with Rett syndrome and 81 pediatric patients with Rett syndrome were pooled for this analysis.

Exposure-response analyses of RSBQ scores were performed on data from pediatric patients with Rett syndrome with available trofinetide exposure estimates, plus placebo patients. All of the patients included in this analysis had at least 1 RSBQ score measured after trofinetide or placebo dosing. The RSBQ E-R analysis dataset included only the 81 pediatric patients with Rett syndrome.

Exposure Measurements

NONMEM (a nonlinear mixed effects modelling tool) was used to generate individual measures of daily trofinetide exposure (Cₘₐₓ, AUC₀₋₁₂, and C_{avg}) via integration of the predicted concentration-time profile for each patient based on the final population PK model and individual empiric Bayesian PK parameter estimates. These time-varying daily trofinetide exposure measures were used in subsequent E-R analyses of efficacy. Exposure measures were set to zero for placebo patients in the E-R analyses.

Pharmacodynamic Sampling Strategy:

The endpoints used for E-R efficacy modeling include CGI-I scores from two clinical studies and RSBQ scores from one clinical study. The CGI-I scores were measured on Days 5, 14, 17, and 26 and CGI-I scores were measured on Days 21, 28, 42, and 54. All patients received placebo for the first 14 days, so Day 14 was treated as the “true baseline” for trofinetide treatment. The RSBQ scores were collected on Days 14, 28, 42 and 54.

Population Pharmacokinetic Analysis Methodology:

Key steps for the development of the trofinetide population PK model were: 1) exploratory data analysis; 2) base structural model development via application, re-estimation, and refinement of a population PK model using pooled data from 9 clinical studies; 3) evaluation of covariate effects using forward selection; 4) full multivariable model evaluation: 5) backward elimination of covariates; 6) final model refinement; and 7) model evaluation. The continuous covariates evaluated included age, body weight, body mass index (BMI), estimated glomerular filtration rate, total bilirubin, alanine aminotransferase, and aspartate aminotransferase. The only categorical covariate evaluated was a combination of sex and disease state (SEXDS), where: SEXDS=0 for male healthy volunteers, SEXDS=1 for female healthy volunteers, SEXDS=2 for patients with Rett (female only), SEXDS=3 for patients with other disease A (male only), SEXDS=4 for male patients with other disease B, and SEXDS=5 for female patients with other disease B. The covariate analysis used a standard forward selection-backward elimination method to identify statistically significant (α=0.001) predictors of PK variability that also explained a sufficient proportion of interindividual variability (IIV) for the respective PK parameter upon which they were tested (that is, reducing IV by ≥5%). The final PK model was validated using a simulation-based, prediction-corrected visual predictive check (pcVPC) methodology to assess concordance between the model-based simulated data and the observed data.

Exposure-Response Analysis Methodology

The overall procedures followed for the development of the E-R models for CGI-I and RSBQ scores were: 1) generation of individual estimates of exposure based on the population PK model; 2) exploratory data analysis; 3) base structural model development incorporating drug exposure effects; 4) evaluation of covariate effects, 5) final model refinement; and 6) model evaluation. Covariates evaluated in the analysis of efficacy endpoints were age, body weight, and BMI. The final E-R efficacy models were validated using a simulation-based, visual predictive check (VPC) methodology to assess concordance between the model-based simulated data and the observed data.

Pediatric Simulations Methodology

A virtual population of 4000 pediatric patients (ages 2 to 5 years) was generated using paired age and body weights from National Health and Nutrition Examination Survey data. Trofinetide concentrations were simulated for the pediatric population following the administration of up to 5 proposed dosing regimens. This was done by randomly sampling from the estimated distributions of PK parameters in the final population PK model. Virtual patients were divided into body weight bands, with different dose amounts administered in each band. Predicted concentration-time profiles were used to compute individual trofinetide exposure estimates (area under the concentration-time curve [AUC], AUC₀₋₁₂, and Cₘₐₓ) in the virtual pediatric population. The pediatric dosing regimen(s) that yielded a range of exposures in the virtual pediatric patients that exhibited the greatest overlap with the target exposure range in patients 5 to 15 years of age were identified.

Population Pharmacokinetic Analysis Results:

A total of 2759 trofinetide plasma concentrations in 281 subjects were used in the development of the population PK model. The analysis dataset comprised subjects with a median (range) age of 22 (5 to 64) years and a median (range) body weight of 65.6 (15.1 to 139.6) kg, with 52.8% of the analysis population being male and 47.2% female.

The final population PK model for trofinetide was a 2-compartment model with first-order absorption, linear elimination, and 2 separate exponential error models for healthy volunteers and patients. The model included separate bioavailability terms for oral dosing (relative bioavailability [F1]) and dosing via gastric tube (F1G). Interindividual variability was estimated for first-order absorption (kₐ), clearance (CL), central volume of distribution (V_{c}), peripheral volume of distribution (Vₚ), and intercompartmental clearance (Q) using exponential error models.

A total of 4 covariate-parameter relationships were included in the final population PK model. Covariate analysis did not identify any additional statistically significant covariate effects that were able to describe at least 5% of IIV in the PK parameter on which it was tested. Therefore, the final PK model included body weight as a significant predictor of both CL (as a power function) and V_{c} (as a power function), and SEXDS as a significant predictor of both CL (as a proportional function) and Vc (as a proportional function).

During the refinement stage, the effect of the SEXDS variable on both CL and V_{c} was further refined by combining relevant categories and evaluating the effect on the precision of the parameter estimates for these covariate relationships with each parameter. After refining the SEXDS covariate-parameter relationships with both CL and Vc, the final population PK model included a proportional shift in CL in patients with one disease and a proportional shift in V_{c} for both Rett patients and patients with another disease.

The final PK model parameter estimates and their associated precisions (relative standard error expressed as a percent [% RSE]) are presented in the table below. All fixed effect parameters were estimated with excellent precision (% RSE≤9.77%). The covariate effect parameters were estimated with reasonable precision (% RSE≤37.8%). All random effect parameters were also estimated with good precision (≤33.5% and ≤11.0% for IIV and RV parameters, respectively).

| | | | Magnitude of | | |
|---|---|---|---|---|---|
| | Final Parameter | | Interindividual | | |
| | Estimate | | Variability | | |
| | Population | | Final | | |
| Parameter | Mean | % RSE | Estimate | | % RES |
| CL: Central Clearance (L/h) | 11.5 | 1.97 | 19.0% | CV | 15.9 |
| CL: Exponent of (WTKG/65) for CL | 0.525 | 6.60 | | | |
| CL: Proportional Shift in CL for disease A = 1 | 0.140 | 33.7 | | | |
| V_{c}: Central Volume (L) | 22.2 | 2.19 | 17.9% | CV | 21.1 |
| V_{c}: Proportional Shift in V_{c} for Rett = 1 | −0.562 | 36.1 | | | |
| V_{c}: Proportional Shift in Vc for disease B = 1 | −0.374 | 37.8 | | | |
| Vc: Exponent of (WTKG/65) for Vc | 0.770 | 14.1 | | | |
| Q: Distribution Clearance (L/h) | 1.02 | 8.09 | 81.9% | CV | 20.0 |
| Vp: Peripheral Volume (L) | 53.0 | 9.77 | 28.6% | CV | 33.5 |
| kₐ: First-Order Absorption Rate Constant (1/h) | 0.195 | 3.96 | 57.8% | CV | 24.8 |
| F1: Oral Bioavailability | 0.770 | 3.63 | NE | | NA |
| F1G: Gastric Tube Bioavailability | 0.847 | 6.52 | NE | | NA |
| Residual Variability HV | 0.0305 | 11.0 | 17.5% | CV | NA |
| Residual Variability Patient | 0.131 | 8.10 | 36.2% | CV | NA |

Minimum Value of the Objective Function = 8393.715
Abbreviations: % CV, coefficient, of variation expressed as a percent; HV, healthy volunteer; NA, not applicable; NE, not estimated; % RSE, relative standard error expressed as a percent; WTKG, weight (kg).

The pcVPC model evaluation indicated that the central tendency in the trofinetide concentration time-course and the magnitude of variability was described reasonably well by the final population PK model.

CGI-I Exposure-Response Analysis Results

The CGI-I E-R analysis dataset included 472 CGI-I scores measured in 133 patients with Rett syndrome. All of the patients in this analysis population were female, with the median age (range) being 14 (5 to 44) years. The median (range) body weight at baseline was 30.4 (15.1 to 79.0) kg. The median CGI-I score in the analysis population was 4, with a minimum score of 2 and a maximum score of 5.

The E-R efficacy model for the CGI-I scores was a proportional odds model with 3 additive components on the logit scale: baseline CGI-I score, placebo time-course, and drug effect. The placebo time-course was an exponential time-course model including parameters estimating rate of change, and a linear function of trofinetide Cₘₐₓ, described the drug effect. None of the covariate effects tested during forward selection resulted in a simultaneous statistically significant improvement of the objective function (α=0.01) and a 5% reduction in IIV.

The final CGI-I E-R model parameter estimates and their associated precisions (% RSE) are presented in the table below. Aside from the marginally high % RSE on the slope for AVGCMAX (% RSE=44.83), the remaining fixed effect parameters were estimated with excellent precision (% RSE≤8.101%). Additive IIV was estimated on the logit function, and was estimated with good precision (% RSE=25.42%).

| | | | Magnitude of | |
|---|---|---|---|---|
| | Final Parameter | | Interindividual | |
| | Estimate | | Variability | |
| | Population | | Final | |
| Parameter | Mean | % RSE | Estimate | % RES |
| LG1: Intercept for CGI-I Score = 2 (logit) | −5.234 | 6.842 | 1.588 SD | 25.42 |
| LG2: Intercept for CGI-I Score = 3 (logit) | 3.319 | 7.813 | NE | NA |
| LG3: Intercept for CGI-I Score = 4 or 5 (Logit) | 5.249 | 8.101 | NE | NA |
| RATE: Exponent of Change in CGI-I Response | 0.01420 | FIXED | NE | NA |
| Due to Time (Logit) | | | | |
| SLP: Slope for AVGCMAX (1/(μg/mL)) | 0.01210 | 44.83 | NE | NA |

Minimum Value of the Objective Function = 836.514
Abbreviations: AVGCMAX, average maximum observed trofinetide concentration; CGI-I, Clinical Global Impression-Improvement; % CV, coefficient of variation expressed as a percent; NA, not applicable; NE, not estimated; % RSE, relative standard error expressed as a percent; SD, standard deviation.

The VPC plots demonstrated that the CGI-I E-R model was reasonable and essentially unbiased, with no significant trends or signs of substantial misfit evident in any of the plots.

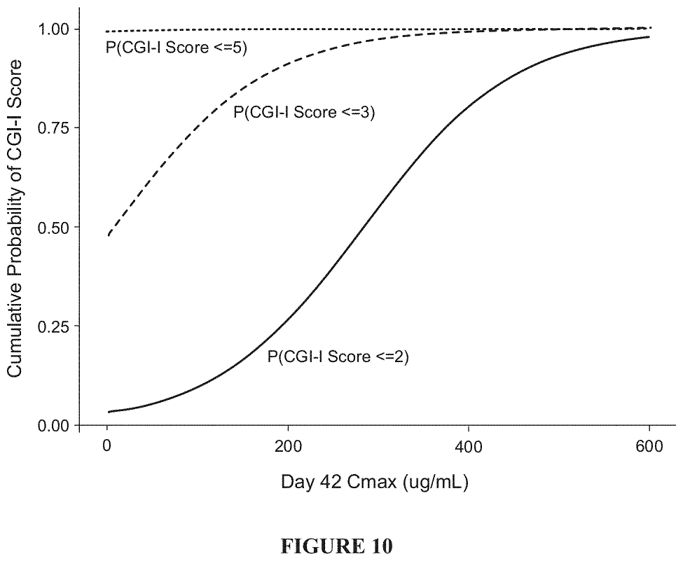

The typical values from the final CGI-1 model were used to generate predicted probabilities of CGI-I scores over a range of Day 42 Cₘₐₓ values of 0 to 600 μg/mL, as shown in FIG. 10. According to the model, for a typical patient on placebo treatment only, the probability of a CGI-I score of 3 (minimally improved) or less is predicted to be 47.5% after 42 days on treatment. While the probability of a CGI-I score of 3 or less by Day 42 of treatment increases to 84.5% in patients treated with trofinetide with an average Day 42 Cₘₐₓ of 150 μg/mL. The probability of a CGI-I score of 2 (much improved) or less by Day 42 of treatment is predicted to be 3.17% in patients treated with placebo and 16.8% in subjects treated with trofinetide with an average Day 42 Ca of 150 μg/mL. These results indicate that a patient treated with trofinetide, who obtained an average Day 42 Cmax of 150 μg/mL, was approximately 1.8 times more likely to exhibit “minimal improvement” from baseline and approximately 5.3 times more likely to exhibit “much improvement” from baseline after 42 days of treatment compared to placebo.

RSBQ Exposure-Response Analysis Results:

The RSBQ E-R analysis dataset included 324 RSBQ scores measured in 81 pediatric patients with Rett syndrome. All of the subjects in this analysis population were female, and the median (range) age of the patients was 9 (5 to 16) years. The median body weight at baseline was 23.3 kg with a range of 15.1 to 62.1 kg. The median (range) baseline RSBQ score in the analysis population was 42 (13 to 69) points. In the placebo group, the median percent change in RSBQ scores from baseline was −5.74% over 54 days. With trofinetide treatment at a dose of 200 mg/kg twice daily (BID), the median percent change in RSBQ scores over the 54 days was −14.4%, indicating greater improvement compared to placebo treatment.

The final E-R model for RSBQ response was a linear time-course model including parameters estimating the baseline RSBQ score and the slope. An exponential function described the relationship between the average daily trofinetide AUC₀₋₁₂ and slope. None of the covariate effects tested during forward selection resulted in a simultaneous statistically significant improvement of the objective function (α=0.01) and a 5% reduction in IIV.

The final RSBQ E-R model parameter estimates and their associated precisions (% RSE) are presented in the table below. The fixed effect parameters were estimated with good precision (% RSE≤18.7%). All random effect parameters were also estimated with good precision (% RSE≤30.6% and % RSE=19.2% for IIV and RV parameters, respectively).

| | | | Magnitude of | |
|---|---|---|---|---|
| | Final Parameter | | Interindividual | |
| | Estimate | | Variability | |
| | Population | | Final | |
| Parameter | Mean | % RSE | Estimate | % RES |
| BL: Baseline RSBQ Score | 41.6 | 3.10 | 11.1 SD | 15.3 |
| SLP: Slope for Time Effect | −0.0527 | FIXED | 0.155 SD | 30.6 |
| SLP: Exponent for AUC₀₋₁₂ | 0.00204 | 18.7 | | |
| on SLP | | | | |
| Additive Residual Error | 14.5 | 19.2 | 3.80 SD | NA |

Minimum Value of the Objective Function = 1548.535
Abbreviations: AUC0-12, area under the concentration-time curve from time 0 to 12 hours; NA, not applicable, RSBQ, Rett Syndrome Behavior Questionnaire; % RSE, relative standard error expressed as a percent; SD, standard deviation.

The VPC plots demonstrated that the RSBQ E-R model was reasonable and essentially unbiased, with no significant trends or signs of substantial misfit evident in any of the plots.

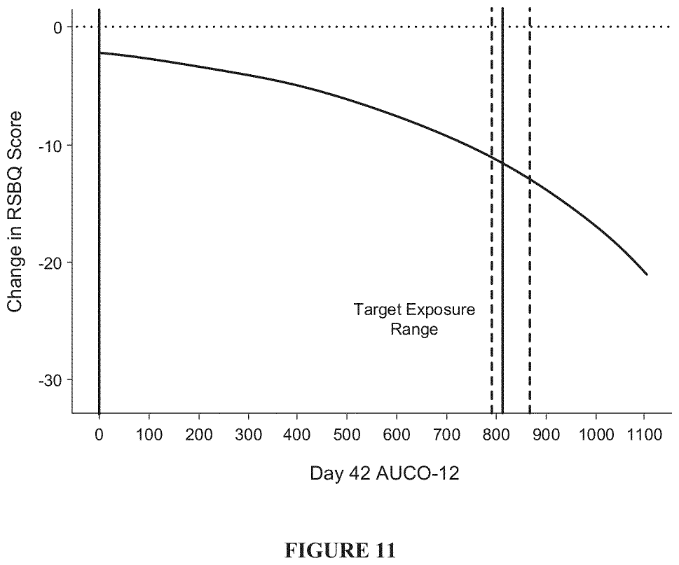

The typical values from the final RSBQ E-R model were used to generate predicted RSBQ scores over a range of area under the concentration-time curve from time 0 to 12 hours at steady state (AUC0-12, ss) values of 0 to 1300 μg×h/mL. FIG. 11 shows the predicted change in RSBQ scores versus AUC₀₋₁₂ on Day 42 of treatment. The dotted line represents a baseline change of zero, the solid lines represent the range of observed exposures in the data used to inform the model, and the dashed lines represent the target steady-state exposure range. According to the model, a typical subject on placebo only would experience an improvement in RSBQ score of −2.21 points from baseline after 42 days, whereas a subject treated with trofinetide for 42 days who obtained an AUC_{0-12,ss} value of 800 μg×h/mL would be expected to have an improvement in their RSBQ score from baseline of −11.3 points. These results indicated that a patient treated with trofinetide with an AUC_{0-12,ss} of 800 μg×h/mL would be expected to experience a 5.1 times greater improvement in their RSBQ score after 42 days of treatment compared to placebo.

Simulation Results

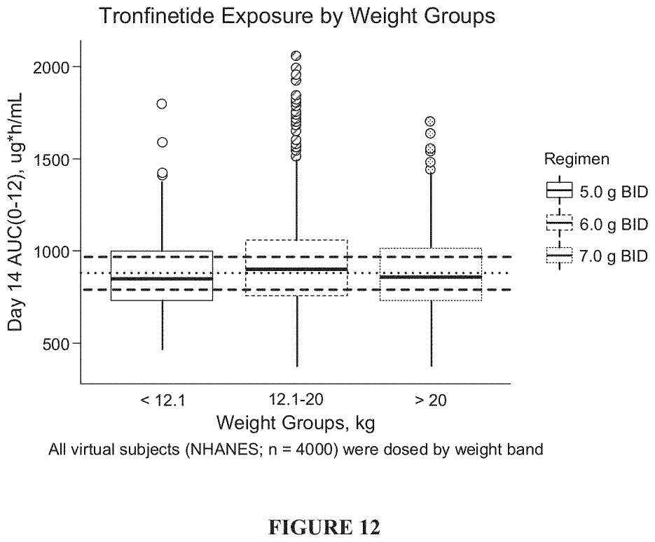

Simulations were performed using the final population PK model described above, and the dose for each weight band that was predicted to result in the greatest overlap with the target exposure range was identified. 5.18 g BID for the 9 to 12 kg body weight band, 5.86 g BID for the >12 to 20 kg body weight band, and 7.19 g BID for the >20 kg body weight band. Since these doses would not be practical in a clinical setting, the doses were rounded to the nearest gram, and the simulations were performed again using the estimated dosing regimens (5.0 g BID for the 9 to 12 kg body weight band, 6.0 g BID for the >12 to 20 kg body weight band, and 7.0 g BID for the >20 kg body weight band). FIG. 12 is a boxplot summarizing the predicted Day 14 AUC₀₋₁₂ values for each body weight band following the estimated dosing regimens (Abbreviations: AUC₀₋₁₂, area under the concentration-time curve from time 0 to 12 hours; BID, twice daily; n, number of subjects; NHANES, National Health and Nutrition Examination Survey).

The estimated doses of 5.0 g BID for the 9 to 12 kg body weight band, 6.0 g BID for the >12 to 20 kg body weight band, and 7.0 g BID for the >20 kg body weight band yielded Day 14 AUC₀₋₁₂ values that overlapped well with the target exposure range of Day 14 AUC₀₋₁₂=790 to 967 μg×h/mL.

Conclusions:

A refined population PK model was developed using PK results of 9 clinical studies with trofinetide. This model confirms that higher trofinetide exposure is associated with greater improvement in Rett syndrome, as measured by CGI-I and RSBQ scores, compared to placebo. The CGI-I model suggests that patients treated with trofinetide, who obtain an average steady-state Cmax of 150 μg/mL, are approximately 1.8 times more likely to exhibit “minimal improvement” from baseline and approximately 5.3 times more likely to exhibit “much improvement” from baseline after 42 days of treatment compared to placebo. The RSBQ model indicates that patients treated with trofinetide, who obtain an AUC_{0-12,ss} of 800 μg×h/mL, would be expected to experience a 5.1 times greater improvement from baseline in their RSBQ score after 42 days of treatment compared to placebo.

### Example 5

### Trofinetide for the Treatment of Girls Two to Five Years of Age with Rett Syndrome

### Primary Objectives

To investigate the safety and tolerability of treatment with oral trofinetide in girls two to five years of age with Rett syndrome and characterize the pharmacokinetics of oral trofinetide in girls two to five years of age with Rett syndrome

### Primary Endpoints

The safety endpoints are as follows: treatment-emergent adverse events (TEAEs); serious adverse events (SAEs); withdrawals due to adverse events; and/or potentially clinically important changes in other safety assessments.

The pharmacokinetic (PK) endpoints are as follows: whole blood concentration of trofinetide and/or trofinetide PK parameters using the population PK approach

### Exploratory Objectives

To investigate the efficacy of treatment with oral trofinetide in girls two to five years of age with Rett syndrome and investigate the benefit of treatment with oral trofinetide on overall quality of life for girls two to five years of age with Rett syndrome

### Exploratory Endpoints

The Exploratory endpoints are as follows: Clinical Global Impression-Improvement (CGI I) Score at Week 12 and End of Treatment (EOT); Caregiver Global Impression-Improvement (CaGI-I) Score at Week 12 and EOT; Clinical Global Impression-Severity (CGI S) change from Baseline to Week 12 and EOT; Caregiver Global Impression-Severity (CaGI-S) change from Baseline to Week 12 and EOT; and/or Overall Quality of Life Rating of the Impact of Childhood Neurologic Disability (ICND) Scale change from Baseline to Weeks 12, 24, 38, 64, 90, and 116

### Test Product, Dose, and Administration

Subjects will begin treatment with trofinetide 2 g BID. The dose will be increased to 3 g BID at the Week 2 visit, 4 g BID at the Week 4 visit, and 5 g BID at the Week 8 visit. In each case, the dose will be increased only if the Investigator judges that the subject is tolerating treatment well. Doses may be orally administered or by gastrostomy (G) tube (doses administered via gastrojejunal [GJ] tubes must be administered through the G-port).

At any point in the study, if the subject cannot tolerate administration of the assigned dose (for example, if the subject experiences diarrhea) the Investigator may instruct the caregiver to reduce the dose of study drug to a dose as low as 1 g BID. In addition, up to four doses (in total, consecutive or non-consecutive) may be withheld within the first 6 weeks. The Investigator will increase the dose as tolerated and will continue treatment on the highest dose the subject can tolerate (up to 5 g BID). This final assigned dose, i.e., the highest tolerated dose, must be given BID and the morning and evening doses must be identical.

### Study Design

This is a multicenter, open-label study of trofinetide for the treatment of girls 2 to 5 years of age with Rett syndrome. Girls 2 to 4 years of age must weigh ≥9 kg. Girls 5 years of age who weigh ≥9 and <12 kg can also be enrolled.

The study will have three main periods:

Screening period: up to 4 weeks

Treatment period
Period A: 12 weeks
Period B: up to approximately 24 months

Safety follow-up period: 30 days

### Screening Period (Up to 4 Weeks)

During the Screening period, subjects will be assessed for study eligibility. Only those subjects who meet all inclusion and no exclusion criteria will be eligible for the study.

Subjects will be evaluated for the diagnosis of Rett syndrome. In addition, there must be verified documentation of a MECP2 mutation.

Investigators should not withdraw a subject's prohibited medication for the purpose of enrolling them into the study. Medications should be discontinued only if it is deemed clinically appropriate to do so and in consultation with the treating physician.

Caregivers will begin to keep a semi-structured caregiver diary during the Screening period.

### Treatment Period A (12 Weeks)

Treatment Period A is designed for evaluation of the dosing, tolerability, and pharmacokinetics of trofinetide in this population. The treatment period is approximately 12 weeks. After Treatment Period A is complete, data will be analyzed.

The Baseline visit (Visit 2) may occur after screening procedures are completed and have not ruled the subject out of eligibility for the study. Eligible subjects will report to the clinical unit for Baseline evaluations on Day −1.

Dosing is twice a day, once in the morning and once in the evening.

The first dose of study drug will be administered after all Baseline assessments are completed, or, if the Investigator judges that it is too late in the day, on the following day. The day the first dose is taken will be considered Day 1 of dosing. A triplicate ECG must be performed 2-3 hours after the first dose and two PK samples will be taken at least 1 hour apart upon completion of the ECG. Study drug must be discontinued in the event that a post-randomization QTcF duration of ≥500 ms or an increase of ≥260 ms compared to the average QTcF interval at Baseline (before dosing) is observed.

After Treatment Period A (and the 30-day follow-up period, if applicable) is completed by all participants, an interim clinical study report of those data will be written. A final clinical study report will be written after the entire study is completed.

### Treatment Period B (Up to Approximately 24 Months)

Treatment Period B is designed to assess the safety and efficacy of long-term treatment with trofinetide.

The dose may be decreased during the study as discussed in the Test Product, Dose, and Administration section above.

During Treatment Period B subjects will have assessments completed at 24 weeks and 38 weeks after the Baseline visit and then every 26 weeks thereafter until the study concludes or is terminated. Assessments may be completed in the clinic or off-site, at the discretion of the Principal Investigator and with the prior approval of the Sponsor or Medical Monitor.

### Safety Follow-Up Period (30 Days)

Caregivers will receive a follow-up telephone call to assess subject safety approximately 30 days after the last dose of study drug if the subject discontinues prematurely or does not enter another trofinetide study.

The schedule of assessments is provided in Table S-1 (Baseline, Screening, and Treatment Period A) and Table S-2 (Treatment Period B and Safety Follow-up).

TABLE S-1

| | Period | | | | | |
|---|---|---|---|---|---|---|
| | Screening | Baseline | Treatment Period A | | | |
| | | Visit | | | | |
| | | Day −1 | Week 2^{a} | Week 4 | Week 8 | Week 12 |
| | Visit Number | | | | | |
| | 1 | 2 | 3 | 4 | 5 | 6 |
| | Visit window (days) | | | | | |
| | N/A | N/A | ±3 | ±3 | ±3 | +3 |
| | Type of Visit^{g} | | | | | |
| | Clinic or Off-site | | | | | |
| Informed consent | X | | | | | |
| Indusion/exclusion criteria | X | X | | | | |
| Medical history and | X | | | | | |
| demographics | | | | | | |
| Confirm documented | X | | | | | |
| Rett diagnosis and MECP2 | | | | | | |
| mutation | | | | | | |
| Rett syndrome history/ | X | | | | | |
| Rett Syndrome Clinical Severity | X | | | | | |
| Scale | | | | | | |
| Physical examination^{g} | X | X | X | X | X | X |
| Vital signs^{b} | X | X | X | X | X | X |
| Height | X | | | | | X |
| Weight | X | X | X^{g} | X^{g} | X^{g} | X^{g} |
| 12-1ead electrocardiogram (ECG)^{c} | X^{c} | X^{c} | X | X | X | X^{c} |
| Clinical laboratory tests | X | X | X | X | X | X |
| Urinalysis | X | X | X | | | X |
| TSH, Free T3, Free T4 | X | | | | | |
| Blood samples for | | X^{d} | X^{e} | X^{e} | X^{e} | X^{e} |
| pharmacokinetics^{d} | | | | | | |
| Clinical Global Impression- | | | X | X | X | X |
| Improvement (CGI-I) | | | | | | |
| Clinical Global Impression- | X | X | X | X | X | X |
| Severity (CGI-S) | | | | | | |
| Caregiver Global Impression- | | | X | X | X | X |
| Improvement (CaGI-I) | | | | | | |
| Caregiver Global Impression- | X | X | X | X | X | X |
| Severity (CaGI-S) | | | | | | |
| Overall Quality of Life Rating | | X | | | | X |
| of the Impact of Childhood | | | | | | |
| Neurologic Disability (ICND) | | | | | | |
| Scale | | | | | | |
| Dispensing and review of semi- | X | X | X | X | X | X |
| caregiver diary | | | | | | |
| Concomitant medications | X | X | X | X | X | X |
| Assessment of adverse events | X | X | X | X | X | X |
| Authorization of study drug | | X------------------------------------------------------X | | | | |
| dispensation^{f} | | | | | | |
| Study drug return^{f} | | | X---------------------------------------X | | | |
| Study drug accountability^{f} | | | X | X | X | X |

Abbreviations:
EOT = end of treatment;
ET = early termination;
TSH = thyroid stimulating hormone
^{a}Timing of post-Baseline visits will be calculated from the first day of dosing (Day 1) (i.e. the Week 2 visit will occur 2 weeks [±3 days] after the first day of dosing).
^{b}Vital signs will include body temperature, resting respiration rate, sitting systolic and diastolic blood pressure, and pulse rate. The sitting blood pressure should be measured after the subject has been sitting for ≥3 minutes.
^{c}ECGs will be completed in triplicate at Visit 1 (Screening), at Visit 2 (Baseline), Day 1 at 2-3 hours after dosing, and at Week 12. A single ECG will be completed at all other designated visits.
^{d}A predose PK blood sample must be collected before administration of study drug at Baseline (Visit 2). After the first dose of study drug is administered, two PK samples wall be collected approximately 1-3 hours after dosing and every effort should be made to separate the two samples by 1 hour.
^{e}At Visit 3, Visit 4, Visit 5, and Visit 6, or upon early termination, two PK samples will be collected at one of the following time intervals: 1-3 hours after dosing, 4-7 hours after dosing, 8-11 hours after dosing. Over the duration of the study, every' effort should be made to collect two PK samples at Visits 3, 4, 5 and 6 within each of the specified time intervals (1-3 hours after dosing, 4-7 hours after dosing, and 8-11 hours after dosing). The two PK samples taken within a time interval should be collected at least one hour apart.
^{f}Investigational product will be shipped directly to the subject. Confirmation of any delivery to the subject will be made by a visiting nurse. Study drug shipment, return, and accountability will be performed in accordance with the drug distribution plan. In addition, study drug will be dispensed at the site during the Baseline visit: when the visit is conducted in the clinic, additional investigational product will be shipped directly to the subject..
^{g}Study visits may be done in the clinic or off-site, at the discretion of the Prinicpal Investigator and with the prior approval of the Sponsor or Medical Monitor. Screening, Baseline, and EOT visits should be done in the clinic whenever possible. When a study visit takes place off-site, the physical examination will not be required. Weight should be measured whenever possible at off-site visits.

TABLE S-2

| | Period | | | | | |
|---|---|---|---|---|---|---|
| | | | | | | Safety |
| | Treatment Period B | | | | | Follow-up |
| | Visit | | | | | |
| | Week | Week | Week | Week | Week 116/ | EOT/ET |
| | 24 | 38 | 64 | 90 | EOT/ET | +30 days |
| | Visit Number | | | | | |
| | 7 | 8 | 9 | 10 | 11 | 8 |
| | Visit window (days) | | | | | |
| | ±7 | ±7 | ±7 | ±7 | ±7 | +4 |
| | Type of Visit^{a} | | | | | |
| | | | | | | Telephone or |
| | Clinic or Off-site | | | | | Telemedicine |
| Physical examination^{a} | X | X | X | X | X | |
| Vital signs^{b} | X | X | X | X | X | |
| Height | | | | | X | |
| Weight | X | X | X^{a} | X^{a} | X^{a} | |
| 12-lead electrocardiogram | X | X | X | X | X | |
| (ECG) | | | | | | |
| Clinical laboratory tests | X | X | X | X | X | |
| Urinalysis | X | X | X | X | X | |
| TSH, Free T3, Free T4 | | | | | | |
| Clinical Global Impression- | X | X | X | X | X | |
| Improvement (CGI-I) | | | | | | |
| Clinical Global Impression- | X | X | X | X | X | |
| Severity (CGI-S) | | | | | | |
| Caregiver Global Impression- | X | X | X | X | X | |
| Improvement (CaGI -I) | | | | | | |
| Caregiver Global Impression- | X | X | X | X | X | |
| Severity (CaGI-S) | | | | | | |
| Overall Quality of Life Rating | X | X | X | X | X | |
| of the Impact of Childhood | | | | | | |
| Neurologic Disability (ICND) | | | | | | |
| Scale | | | | | | |
| Dispensing and review of | X | X | X | X | | |
| semi- caregiver diary | | | | | | |
| Concomitant medications | X | X | X | X | X | X |
| Assessment of adverse events | X | X | X | X | X | X |
| Authorization of study drug | X-----------------------------------------------------X | | | | | |
| dispensation^{c} | | | | | | |
| Study drug return^{c} | X-----------------------------------------------------X | | | | | |
| Study drug accountability^{c} | X | X | X | X | X | |

Abbreviations:
EOT = end of treatment;
ET = early termination;
TSH = thyroid stimulating hormone
^{a}Study visits may be done in the clinic or off-site, at the discretion of the Prinicpal Investigator and with the prior approval of the Sponsor or Medical Monitor. Screening, Baseline, and EOT visits should be done in the clinic whenever possible. When a study visit takes place off-site, the physical examination will not be required. Weight should be measured whenever possible at off-site visits.
^{b}Vital signs wall include body temperature, resting respiration rate, sitting systolic and diastolic blood pressure, and pulse rate. The sitting blood pressure should be measured after the subject has been sitting for ≥3 minutes.
^{c}Investigational product will be shipped directly to the subject. Confirmation of any delivery to the subject will be made by a visiting nurse. Study drug shipment, return, and accountability will be performed in accordance with the drug distribution plan. In addition, study drug will be dispensed at the site during the Baseline visit when the visit is conducted in the clinic, additional investigational product will be shipped directly to the subject.

### Study Duration

The duration of participation for individual study subjects will be approximately 29 months, consisting of a screening period of up to 4 weeks, a 12-week treatment period, an approximately 24-month treatment period, and a safety follow-up period of 30 days.

The study completion date is defined as the date the final subject, across all sites, completes their final protocol-defined assessment.

Main Criteria for Inclusion and Exclusion To be eligible for this study, subjects must meet all of the inclusion criteria and none of the exclusion criteria.

### Inclusion Criteria:

1. Informed consent prior to the conduct of any study procedures is required as follows:

a. Written informed consent will be obtained from the legally acceptable representative (LAR). The process of obtaining informed consent will be conducted in accordance with institutional review board (IRB) or ethics committee (EC) policy and applicable local law.

b. The subject's caregiver must also provide written informed consent regarding their participation in the study prior to participating in any study procedures.

2. Female subject

a. 2 to 4 years of age and body weight ≥9 kg at Screening OR

b. 5 years of age and body weight ≥9 kg and <12 kg

3. Can swallow the study medication provided as a liquid solution or can take it by gastrostomy tube

4. The subject's caregiver is English-speaking and has sufficient language skills to complete the caregiver assessments

### Diagnosis

5. Has classic/typical Rett syndrome (RTT) or possible RTT according to the Rett Syndrome Diagnostic Criteria (Appendix A)

6. Has a documented disease-causing mutation in the MECP2 gene

7. Has a CGI-S score of 24 at Screening and Baseline

Concomitant Treatment

8. If the subject is taking or was taking an anticonvulsant or any other psychoactive medication (including cannabinoids):

a. the treatment regimen has been stable for at least 4 weeks before Baseline and there is no current plan to change the dose, OR

b. if the medication was discontinued, the discontinuation has occurred no fewer than 2 weeks or 5 half-lives (whichever is greater) before Baseline

9. If the subject is taking or was taking any other medication daily for a chronic illness (not including antibiotics, pain relievers, and laxatives):

a. the treatment regimen of the medication has been stable for at least 4 weeks before Baseline and there is no current plan to change the dose, OR

b. if the medication was discontinued, the discontinuation has occurred no fewer than 2 weeks or 5 half-lives (whichever is greater) before Baseline

10. If the subject is receiving or was receiving a non-pharmacologic somatic treatment (e.g., a ketogenic diet or vagal nerve stimulation):

a. the treatment regimen has been stable for at least 4 weeks before Baseline and there is no current plan to change the treatment, OR

b. if the treatment was discontinued, the discontinuation has occurred no fewer than 2 weeks before Baseline

11. If the subject is receiving or was receiving non-pharmacologic treatments such as an educational, behavioral, physical, or speech therapy:

a. the treatment regimen has been stable for at least 4 weeks before Baseline and there is no current plan to change the treatment (Note: changes to a treatment regimen that are due to school schedules or are otherwise seasonally related are not exclusionary), OR

b. if the treatment was discontinued, the discontinuation has occurred no fewer than 2 weeks before Baseline

### Seizures

12. Has a stable pattern of seizures, or has had no seizures, within 8 weeks of Screening

### Place of Residence

13. Subject and caregiver(s) must reside at a location to which study drug can be delivered and have been at their present residence for at least 3 months prior to Screening

Exclusion Criteria:

Concomitant Treatment

1. Has been treated with growth hormone within 12 weeks of Baseline

2. Has been treated with IGF-1 within 12 weeks of Baseline

3. Has been treated with insulin within 12 weeks of Baseline

Medical Conditions Other than Rett Syndrome

4. Has current clinically significant cardiovascular, endocrine (such as hypo- or hyperthyroidism, Type 1 diabetes mellitus, or uncontrolled Type 2 diabetes mellitus), renal, hepatic, respiratory or gastrointestinal disease (such as celiac disease or inflammatory bowel disease) or has major surgery planned during the study

5. Has a history of, or current, cerebrovascular disease or brain trauma

6. Has significant, uncorrected visual or uncorrected hearing impairment

7. Has a history of, or current, malignancy

Laboratory Studies, Vital Signs, and Electrocardiogram

8. Has one or more clinical laboratory test value(s) outside the range specified below at Screening:

a. hemoglobin below the normal range

b. aspartate aminotransferase (AT) or alanine aminotransferase (ALT) value more than 1.5× the upper limit of the normal range (ULN) for that subject's age and gender

c. total bilirubin value >25.7 μmol/L (1.5 mg/dL)

d. Serum creatinine ≥ULN or a calculated creatinine clearance <90 mL/minute (CLcr) (using the revised Schwartz estimate of creatinine clearance; Schwartz et al. 2009, http://www-users.med.cornell.edu/-spon/picu/calc/crclsch2.htm)

e. serum potassium below the normal range; serum potassium may be repeated during the Screening period with the agreement of the Medical Monitor

f. hemoglobin A1C (HbA1c) >7.0%

g. thyroid stimulating hormone (TSH) value outside the normal range

9. Has a clinically significant abnormal laboratory value at Screening. Laboratory testing may be repeated during the Screening period with agreement of the Medical Monitor.

10. Has clinically significant abnormality in vital signs at Screening or Baseline

11. Has any of the following:

a. QTcF interval of >450 ms at Screening or Baseline

b. History of a risk factor for torsades de pointes (e.g., heart failure or family history of long QT syndrome)

c. History of clinically significant QT prolongation that is deemed to put the subject at increased risk of clinically significant QT prolongation

d. Other clinically significant finding on ECG at Screening or Baseline

Other Criteria

12. Has a significant sensitivity or allergic reaction to trofinetide or its excipients

13. Has participated in another interventional clinical study within 30 days prior to Screening

14. Is judged by the Investigator or the Medical Monitor to be inappropriate for the study for any reason

Pharmacokinetic and other assessments will be made according to the procedures set forth in Example 2.

Having now fully described the compounds, compositions, and methods herein, it will be understood by those of skill in the art that the same can be performed within a wide and equivalent range of conditions, formulations, and other parameters without affecting the scope of the compounds, compositions, and methods provided herein, or any embodiment thereof.

All patents, patent applications, and publications cited herein are fully incorporated by reference herein in their entirety

### LITERATURE REFERENCES

Amir R E, Van den Veyver I B, Schultz R, et al. Influence of mutation type and X chromosome inactivation on Rett syndrome phenotypes. Ann Neurol. 2000; 47:670-679.
Anagnostou E, Jones N, Huerta M, et al. Measuring Social Communication Behaviors as a Treatment Endpoint in Individuals with Autism Spectrum Disorder. Autism. 2015;19(5):622-36.
Barnes K V, Coughlin F R, O'Leary H M, et al. Anxiety-like behaviour in Rett syndrome: characteristics and assessment by anxiety scales. J Neurodev Disord. 2015:7(1):30.
Bienvenu T, Philippe C, De Roux N, et al. The incidence of Rett syndrome in France. Pediatr Neurol. 2006; 34(5):372-375.
Buchovecky C M, Turley S D, Brown H M, et al. A suppressor screen in Mecp2 mutant mice implicates cholesterol metabolism in Rett syndrome. Nat Genet. 2013; 45(9):1013-1020.
Busner J, Targum S D. The clinical global impressions scale: applying a research tool in clinical practice. Psychiatry. 2007;July;4(7):28-37.
Byiers B J, Symons F J. Issues in estimating developmental level and cognitive function in Rett syndrome. In: Hodapp R M ed. International Review of Research in Intellectual and Developmental Disabilities. Waltham, Mass.: Elsevier Inc.; 2012; 43:147-185.
Camfield C, Breau L, Camfield P. Assessing the impact of pediatric epilepsy and concomitant behavioral, cognitive, and physical/neurologic disability: Impact of Childhood Neurologic Disability Scale. Dev Med Child Neurol. 2003; 45:152-159.
Cass H, Reilly S, Owen L, et al. Findings from a multidisciplinary clinical case series of females with Rett syndrome. Dev Med Child Neurol. 2003; 45:325-337.
Cianfaglione R, Clarke A, Kerr M, et al. A national survey of Rett syndrome: behavioural characteristics. J Neurodev Disord. 2015;7(1):11.
Cianfaglione R, Clarke A, Kerr M, Hastings R P, Oliver C, Felce D. Ageing in Rett syndrome. J Intellect Disabil Res. 2016; 60(2):182-190.
Clarkson T, LeBlanc J, DeGregorio G, et al. Adapting the Mullen Scales of Early Learning for a standardized measure of cognition in children with Rett syndrome. Intellect Dev Disabil. 2017; 55(6):419-431.
Djukic A, McDermott M V. Social preferences in Rett syndrome. Pediatr Neurol. 2012;46(4):240-242.
Downs J, Bebbington A, Jacoby P, et al. Level of purposeful hand function as a marker of clinical severity in Rett syndrome. Dev Med Child Neurol. 2010; 52(9):817-823.
Downs J, Bebbington A, Kaufmann W E, Leonard H. Longitudinal hand function in Rett syndrome. J Child Neurol. 2011:26(3):334-340.
Ehrhart F, Coort S L, Cirillo E, Smeets E, Evelo C T, Curfs L. New insights in Rett syndrome using pathway analysis for transcriptomics data. Wien Med Wochenschr. 2016; 166:346-352.
Epstein A, Leonard H, Davis E, et al. Conceptualizing a quality of life framework for girls with Rett syndrome using qualitative methods. Am J Med Genet A. 2016; 170(3):645-653.
Glaze D G, Neul J L, Kaufmann W E, et al. Double-blind, randomized, placebo-controlled study of trofinetide in pediatric Rett syndrome. Neurology. 2019 Mar. 27. pii: 10.1212/WNL.0000000000007316. [Epub ahead of print].
Glaze D G, Neul J L, Percy A, et al. A double-blind, randomized, placebo-controlled clinical study of trofinetide in the treatment of Rett syndrome. Pediatr Neurol. 2017; 76:37-46.
Glaze D G, Percy A K, Skinner S, et al. Epilepsy and the natural history of Rett syndrome. Neurology. 2010; 74:909-912.
Hite K C, Adams V H, Hansen J C. Recent advances in MeCP2 structure and function. Biochem Cell Biol. 2009;87(1):219-227.
ICH Harmonized Tripartite Guideline: Clinical Safety Data Management: Definitions and Standards for Expedited Reporting E2A. International Conference on Harmonisation of Technical Requirements for Registration of Pharmaceuticals for Human Use. October 1994.
Iemmi V, Knapp M, Brown F J. Positive behavioural support for children and adolescents with intellectual disabilities whose behaviour challenges: an exploration of the economic case. J Intellect Disabil. 2015; 20(3):281-295.
Ip J P K, Mellios N, Sur M. Rett syndrome: insights into genetic, molecular and circuit mechanisms. Nat Rev Neurosci. 2018; 19(6):368-382.
Jusko W J, Chiang S T. Distribution volume related to body weight and protein binding. J Pharm Sci. 1982; 71(4):469-70.
Kaufmann W E, Tierney E, Rohde C A, et al. Social impairments in Rett syndrome: characteristics and relationship to clinical severity. J Intellect Disabil Res. 2012; 56(3):233-247.
Kerr A M, Armstrong D D, Prescott R J, Doyle D, Kearney D L. Rett syndrome: analysis of deaths in the British survey. Eur Child Adolesc Psychiatry. 1997;6 Suppl 1:71-74.
Kersting G, Willmann S, Würthwein G, et al. Physiologically based pharmacokinetic modelling of high- and low-dose etoposide: from adults to children. Cancer Chemother Pharmacol. 2012 February;69(2).397-405.
Khwaja O, Ho E, Barnes K V, et al. Safety, pharmacokinetics, and preliminary assessment of efficacy of mecasermin (recombinant human IGF-1) for the treatment of Rett syndrome. Proc Natl Acad Sci U.S.A. 2014; 111(12):4596-4601.
Kriaucionis S, Bird A. DNA methylation and Rett syndrome. Hum Mol Genet. 2003;12 Spec No 2:R221-227.
Krishnaraj R, Ho G, Christodoulou J. RettBASE: Rett syndrome database update. Hum Mutat. 2017; 38(8):922-931.
Lane J, Salter A, Jones N E, et al. Assessment of Caregiver Inventory for Rett syndrome. J Autism Dev Disord. 2017;47(4). 1102-1112.
Larsson G, Lindstrom B, Witt Engerström I. Rett syndrome from a family perspective: the Swedish Rett Center Survey. Brain Dev. 2005;27 Suppl 1:S14-19.
Lee J Y, Leonard H, Piek J P, Downs J. Early development and regression in Rett syndrome. Clin Gentet. 2013; 84(6):572-576.
Monrós E, Armstrong J, Aibar E, Poo P, Canos I, Pineda M. Rett syndrome in Spain: mutation analysis and clinical correlations. Brain & Development. 2001;23:S251-S253.
Motil K J, Caeg E, Barrish J O, et al. Gastrointestinal and nutritional problems occur frequently throughout life in girls and women with Rett syndrome. J Pediatr Gastroenterol Nutr. 2012; 55(3):292-298.
Mount R H, Charman T, Hastings R P, Reilly S, Cass H. Features of autism in Rett syndrome and severe mental retardation. J Autism Dev Disord. 2003; 33(4):435-442.
Mount R H, Charman T, Hastings R P, Reilly S, Cass H. The Rett Syndrome Behaviour Questionnaire (RSBQ): Refining the behavioural phenotype of Rett syndrome. J Child Psychol Psychiatry. 2002; 43(8):1099-1110.
Mount R H, Hastings R P, Reilly S, Cass H, Charman T. Behavioural and emotional features in Rett syndrome. Disabil Rehabil. 2001;23(3-4):129-138.
Neul J L, Benke T A, Marsh E D, et al. The array of clinical phenotypes of males with mutations in Methyl-CpG binding protein 2. Am J Med Genet B Neuropsychiatr Genet. 2019; 180(1):55-67.
Neul J L, Glaze D, Percy A, et al. Improving Treatment Trial Outcomes for Rett Syndrome: the development of Rett-specific anchors for the Clinical Global Impression Scale. J Child Neurol. 2015; 30(13):1743-1748.
Neul J L, Lane J B, Lee H S, et al. Developmental delay in Rett syndrome: data from the natural history study. J Neurodevelop Dis. 2014;(30):2-9.
Neul J L, Kaufmann W E, Glaze D G, et al. Rett syndrome: revised diagnostic criteria and nomenclature. Ann Neurol. 2010; 68(6):944-950.
Neul J L, Fang P, Barrish J, et al. Specific mutations in methyl-CpG-binding protein 2 confer different severity in Rett syndrome. Neurology. 2008; 70(16):1313-1321.
Novak M, Guest C. Application of a multidimensional Caregiver Burden Inventory. Gerontologist. 1989; 29(6):798-803.
O'Leary H M, Kaufmann W E, Barnes K V, et al. Placebo controlled crossover efficacy assessment of mecasermin for the treatment of Rett syndrome. Ann Clin Transl Neurol 2018; 5(3):323-332.
Oosterholt S P, Horrigan J, Jones N, Glass L, Della Pasqua O. Population pharmacokinetics of NNZ-2566 in healthy subjects. Eur J Pharm Sci. 2017;15;109S:S98-107.
Palacios-Cefia D, Famoso-Perez P, Salom-Moreno J, et al. “Living an Obstacle Course”: A Qualitative Study Examining the Experiences of Caregivers of Children with Rett Syndrome. Int. J Environ Res Public Health. 2018;16(1):41.
Percy A K, Lane J, Annese F, Warren H, Skinner S A, Neul J L. When Rett syndrome is due to genes other than MECP2. Transl Sci Rare Dis. 2018; 3(1):49-53.
Percy A K, Lee H S, Neul J L, et al. Profiling scoliosis in Rett syndrome. Pediatr Res. 2010;67(4):435-439.
Perry A, Sarlo-McGarvey, Haddad, C. Brief report: cognitive and adaptive functioning in 28 girls with Rett syndrome. J Autism Dev Disord. 1991; 21(4):551-556.
Piana C, Zhao W, Adkison K, et al. Covariate effects and population pharmacokinetics of lamivudine in HIV-infected children. Br J Clin Pharmacol. 2014; 77(5):861-72.
Robertson L, Hall S E, Jacoby P, Ellaway C, de Klerk N, Leonard H. The association between behaviour and genotype in Rett syndrome using the Australian Rett syndrome database. Am J Med Genet. 2006;141B(2):177-183.
Rose S A, Djukic A, Jankowski J J, Feldman J F, Fishman 1, Valicenti-McDemott M. Rett syndrome: an eye-tracking study of attention and recognition memory. Dev Med Child Neurol. 2013; 55(4):364-371.
Samaco R C, Neul J L. Complexities of Rett syndrome and MeCP2. J Neurosci. 2011; 31(22):7951-7959.
Shovlin S, Tropea D. Transcriptome level analysis in Rett syndrome using human samples from different tissues. Orphanet J Rare Dis. 2018;13(1):113.
Thompson R H, Iwata B A. A descriptive analysis of social consequences following problem behavior. J Appl Behav. Anal. 2001; 34(2):169-178.
Tropea D, Giacomettim E, Wilson N R, et al. Partial reversal of Rett Syndrome-like symptoms in MeCP2 mutant mice. PNAS. 2009; 106:2029-2034.
Urbanowicz A, Downs J, Girdler S J, Ciccone N A, Leonard H. An exploration of the use of eye gaze and gestures in females with Rett syndrome. J Speech Lang Hear Res. 2016:59(6):1373-1383.
Urbanowicz A, Downs J, Girdler S J, Ciccone N A, Leonard H. Aspects of speech language abilities are influenced by MECP2 mutation type in girls with Rett syndrome. Am J Med Genet. 2015;167A(2):354-362.
West P R, Amaral D G, Bais P, et al. Metabolomics as a tool for discovery of biomarkers of autism spectrum disorder in the blood plasma of children. PLoS One. 2014;9(11):e112445.
Wetherby A M, Guthrie W, Woods J, et al. Parent-implemented social intervention for toddlers with autism: an RCT. Pediatrics. 2014; 134(6):1084-1093.
Wetherby A M, Allen L, Cleary J, Kublin K, Goldstein H. Validity and reliability of the communication and symbolic behavior scales developmental profile with very young children. J Speech Lang Hear Res. 2002; 45(6):1202-1218.
Woodyatt G C, Ozanne A E. A longitudinal study of cognitive skills and communication behaviours in children with Rett syndrome. J Intellect Disabil Res. 1993;37(Pt 4):419-435.
Yasui D H, Xu H, Dunaway K W, Lasalle J M, Jin L W, Maezawa I. MeCP2 modulates gene expression pathways in astrocytes. Mol Autism. 2013;4(1):3.
Young D J, Bebbington A, Anderson A, et al. The diagnosis of autism in a female: could it be Rett syndrome? Eur J Pediatr. 2008; 167(6):661-669.

1. A method of treating Rett syndrome in a subject in need thereof, the method comprising administering a therapeutically effective amount of trofinetide to the subject in a daily amount of:
a) 4-10.0 g if the subject weighs between 8-11.9 kg;
b) 10.1-14.0 g if the subject weighs between 12.0-20.0 kg;
c) 14.1-18.0 g if the subject weighs between 20.1-35.0 kg;
d) 18.1-22.0 g if the subject weighs between 35.1-50.0 kg; or
e) 22.1-26 g if the subject weighs between 50.1-150 kg.

2. The method of claim 1, wherein the subject weighs between 8-11.9 kg and trofinetide is administered in a daily amount of 4 g, about 5 g, about 6 g, about 7 g, about 8 g, about 9 g, or 10 g.

3. (canceled)

4. The method of claim 1, wherein the subject weighs between 12.0-20.0 kg and trofinetide is administered in a daily amount of about 12 g.

5. (canceled)

6. The method of claim 1, wherein the subject weighs between 20.1-35.0 kg and trofinetide is administered in a daily amount of about 16 g.

7. (canceled)

8. The method of claim 1, wherein the subject weighs between 35.1-50.0 kg and trofinetide is administered in a daily amount of about 20 g.

9. (canceled)

10. The method of claim 1, wherein the subject weighs between 50.1-100 kg and trofinetide is administered in a daily amount of about 24 g.

11. (canceled)

12. The method of claim 1, wherein the administration of trofinetide results in an AUC_{0-12, ss} of about 755 μg·h/mL to about 1300 μg·h/mL in the subject.

13. (canceled)

14. The method of claim 1, wherein the administration of trofinetide to the subject in a C_{max, ss} of about 100 to about 200 μg/mL in the subject.

15. (canceled)

16. (canceled)

17. The method of claim 1, wherein trofinetide is administered to the subject in multiple doses per day that sum to the daily amount.

18. (canceled)

19. The method of claim 1, wherein trofinetide is administered as a pharmaceutical composition comprising trofinetide and a pharmaceutically acceptable carrier, the pharmaceutically acceptable carrier comprises water, the pharmaceutical composition is a solution, the concentration of trofinetide in the solution is about 0.05 g/mL-0.7 g/mL, and the solution is orally administered to the subject.

20. (canceled)

21. (canceled)

22. (canceled)

23. (canceled)

24. (canceled)

25. (canceled)

26. (canceled)

27. (canceled)

28. The method of claim 1, wherein the subject has a MECP2 mutation.

29. The method of claim 1, wherein the Rett syndrome is atypical Rett syndrome.

30. The method of claim 1, wherein the Rett syndrome is classic/typical Rett syndrome.

31-90. (canceled)

91. A method of treating Rett syndrome in a subject in need thereof, the method comprising administering to the subject a daily amount of trofinetide that provides an AUC_{0-12, ss} of about 755 μg·h/mL to about 1300 μg·h/mL in the subject.

92. (canceled)

93. (canceled)

94. The method of claim 91, wherein the subject weighs between about 12 kg to about 20 kg and the daily amount of trofinetide is about 12 g.

95. (canceled)

96. The method of claim 91, wherein the subject weighs between about 20.1 kg to about 35 kg and the daily amount of trofinetide is about 16 g.

97. (canceled)

98. The method of claim 91, wherein the subject weighs between about 35.1 kg to about 50 kg and the daily amount of trofinetide is about 20 g.

99. (canceled)

100. The method of claim 91, wherein the subject weighs between about 50.1 kg to about 100 kg and the daily amount of trofinetide is about 24 g.

101. (canceled)

102. (canceled)

103. The method of claim 91, wherein trofinetide is administered to the subject in multiple doses per day that sum to the daily amount.

104. (canceled)

105. The method of claim 91, wherein trofinetide is administered as a pharmaceutical composition comprising trofinetide and a pharmaceutically acceptable carrier, the pharmaceutically acceptable carrier comprises water, the pharmaceutical composition is a solution, the concentration of trofinetide in the solution is about 0.05 g/mL-0.7 g/mL, and the solution is orally administered to the subject.

106-113. (canceled)

114. The method of claim 91, wherein the subject has a MECP2 mutation.

115. The method of claim 91, wherein the Rett syndrome is atypical Rett syndrome.

116. The method of claim 91, wherein the Rett syndrome is classic/typical Rett syndrome.

117-120. (canceled)
